SEQUENCE LISTING (5 sequences)

SEQ ID NO: 1 - DNA, 25 bp, Artificial Sequence
  Description of Artificial Sequence Synthetic primer
ggtcgtactgcgtatcctgcctcag

SEQ ID NO: 2 - DNA, 24 bp, Artificial Sequence
  Description of Artificial Sequence Synthetic primer
taccatgatcttgagaagtccatg

SEQ ID NO: 3 - DNA, 20 bp, Artificial Sequence
  Description of Artificial Sequence Synthetic primer
cttgggtggagaggctattc

SEQ ID NO: 4 - DNA, 20 bp, Artificial Sequence
  Description of Artificial Sequence Synthetic primer
aggtgagatgacaggagatc

SEQ ID NO: 5 - protein, 4 aa, Artificial Sequence
  Description of Artificial Sequence Synthetic peptide
KDEL

(19) United States
(12) Patent Application Publication
(10) Pub. No.: US 2009/0041753 A1
(43) Pub. Date: Feb. 12, 2009

(54) Cytokine signaling

(76) Inventors: Robert H. Miller, Cleveland Heights, OH (US); Dolly A. Padovani-Claudio, Cleveland, OH (US)

Correspondence Address: WILSON SONSINI GOODRICH & ROSATI, 650 PAGE MILL ROAD, PALO ALTO, CA, 94304-1050, US

(21) Appl. No.: 11/904,634

(22) Filed: Sep. 26, 2007

Related U.S. Application Data
(60) Provisional application No. 60/847,656, filed on Sep. 26, 2006.

(51) Int. Cl. A61K 39/395; A61K 31/4965; A61K 31/54; A61K 31/517; A61P 25/00; A61K 31/7088; A61K 31/53; A61K 38/16

(52) U.S. Cl. 424/130.1; 514/255.02; 514/226.5; 514/266.3; 514/246; 514/12; 514/2; 514/44

(57) ABSTRACT

The present invention relates to compositions and methods for targeting CXC-chemokine mediated signaling for treatment of a myelin disorder.

### CROSS-REFERENCE

This application claims the benefit of U.S. Provisional Application No. 60/847,656, filed Sep. 26, 2006, which is incorporated herein by reference in its entirety.

### STATEMENT AS TO FEDERALLY SPONSORED RESEARCH

This invention was made with the support of the United States government under Grant number NS36674, NS47928, and NS32151 by the National Institute of Health.

### BACKGROUND OF THE INVENTION

Multiple sclerosis (MS) is a demyelinating disease of the central nervous system (CNS) with clinical deficits ranging from relapsing-remitting to chronic-progressive patterns of expression. Although the etiology of MS is unknown, it is recognized that autoreactive CD4⁺ T cell responses mediate inflammatory damage against myelin and oligodendrocytes. (Bruck et al., J. Neurol. Sci. 206:181-185 (2003)). CNS lesions have focal areas of myelin damage and are also associated with axonal pathology, neuronal distress, and astroglial scar formation. (Compston et al., Lancet. 359:1221-1231 (2002)). Clinical presentation includes various neurological dysfunctions including blindness, paralysis, loss of sensation, as well as coordination and cognitive deficits. Furthermore, damage or injury to myelin has severe consequences on conduction velocity and the vulnerability of neurons to axonal destruction. In addition, there is a correlation between axon loss and progressive clinical disability and intact myelin is important in the maintenance of axonal integrity. (Dubois-Dalcq et al., Neuron. 48, 9-12 (2005)). Spontaneous remyelination may occur during the early phases of human MS, however, persistent CNS inflammation and the failure of myelin repair during later stages of the disease ultimately lead to permanent debilitation. Some reasons for remyelination failure may include: 1) lack of oligodendrocyte survival, 2) lack of oligodendrocyte progenitor proliferation, 3) inability of progenitors to migrate to the lesion site, and/or 4) inability of progenitors to differentiate into mature myelinating cells or to ensheath denuded axons. (Franklin, Nat. Rev. Neurosci. 3:705-714 (2002))

Adult oligodendrocyte progenitor cells are generally believed to be responsible for remyelination, and thus, the failure of remyelination is, at least in part, associated with deficiencies in the generation of mature oligodendrocytes, leading to inability to myelinate. Myelination relies on the coordination of multiple signals including those that precisely localize oligodendrocytes and their precursors (Tsai et al., Cell 110:373-383 (2002); Tsai et al., J. Neurosci. 26:1913-1922 (2006)). For example, Netrin-1 is required for the normal development of spinal cord oligodendrocytes (Tsai et al., J. Neurosci. 26:1913-1922 (2006)), regulation of appropriate cell numbers (Barres et al., Cell 70:31-46 (1992); Calver et al., Neuron 20:869-882 (1998)), and mediation of interactions between oligodendrocytes and their target axons. (Sherman and Brophy, Nat. Rev. Neurosci. 6:683-690 (2005)). Oligodendrocyte progenitor cells (OPCs) have extensive arborizations and are found ubiquitously in the adult CNS (Baumann and Pham-Dinh, Physiol. Rev. 81:871-927 (2001)). Furthermore, OPCs may be characterized in vivo by the expression of the antigenic markers NG2 and PDGFRα, and in vitro by their A2B5 expression. Evidence suggests axonal atrophy is highly correlated with a declining clinical outcome (e.g., effects of demyelination on axonal survival).

In order to ameliorate disability in MS, remyelination aims to restore axonal conduction and prevent further axonal damage that could arise in denuded axons.

Proliferation can provide enough cells to myelinate developing axons (Baumann and Pham-Dinh, Physiol. Rev. 81:871-927 (2001)). Furthermore, remyelinated areas of white matter exhibit a higher number of oligodendrocytes than non-demyelinated white matter (Franklin. Nat. Rev. Neurosci. 3:705-714 (2002)), indicating the need for recruitment of cells for an effective remyelinative process. Although OPCs are considered to be progenitors in the adult CNS despite their morphological resemblance to astrocytes and microglia, as well as the fact that they express a variety of ion channels and neurotransmitter receptors, these cells have been shown to have functional and antigenic characteristics distinct from astrocytes (Nishiyama et al., J. Neurocytol. 31:437-455 (2002)). In addition, OPCs appear to be different from microglia and neurons (Nishiyama et al., Hum. Cell 14:77-82 (2001)). OPCs have the capacity to react quickly to injury through enhanced migration and differentiation into oligodendrocytes in both the normal and injured CNS after extensive migration (Baumann and Pham-Dinh, Physiol. Rev. 81:871-927 (2001); Polito and Reynolds, J. Anat. 207:707-716 (2005)).

OPCs express the chemokine receptor CXCR2, while its ligand, CXCL1, is produced by astrocytes, microglia, and a subset of neurons. MS plaque repopulation and remyelination is typically dependent on adult OPCs, and CXCR2 signaling can modulate OPC proliferation and migration (Robinson et al., Neurosurgery 48:864-874 (2001); Robinson et al., J. Neurosci. 18:10457-10463 (1998); Tsai et al., Cell 110:373-383 (2002); Wu et al., J. Neurosci. 20:2609-2617 (2000)).

Chemokines or chemoattractant cytokines comprise a family of inducible secreted molecules of small molecular weight (˜8-10 KDa) (Hesselgesser and Horuk, J. Neurovirol. 5:13-26 (1999)) which typically function as activators and chemoattractants to leukocytes (Coughlan et al., Neuroscience 97:591-600 (2000); Ransohoff and Tani, Trends Neurosci. 21:154-159 (1998)) and can modulate angiogenesis (Ueda et al., Cancer Res. 66:5346-5353 (2006)), wound healing (Devalaraja et al., J. Invest. Dermatol. 115:234-244 (2000)), and tumorigenesis (Loukinova et al., Int. J. Cancer 94):637-644 (2001); Robinson et al., Neurosurgery 48:864-734 (2001)). Most of the knowledge of chemokine functions is derived from research on the immune system due to their implication in regulation of immune coordination and inflammation. Roles involving many other biological systems are slowly being elucidated.

Chemokines are typically classified into four subfamilies according to the number of conserved cysteine residues in their amino terminus (Nomiyama et al., Genes Immun. 2:110-113 (2001)). Most chemokines fit into two main subfamilies with four cysteine residues. These subfamilies are typically classified according to the presence or absence of an amino acid between the two amino terminus cysteine residues, and are thus named CC and CXC chemokines (Hesselgesser and Horuk, J. Neurovirol. 5:13-26 (1999)). CXC chemokines, which are typically restricted to higher vertebrates, are usually further classified according to the presence or absence of a glutamate-lysine-arginine (ELR) motif on their amino terminus adjacent to the first cysteine residue. CXCL1, previously known as Gro-α, is a member of the ELR family of CXC chemokines whose preferred receptor is CXCR2 (Wang et al., Biochemistry 42:1071-1077 (2003)).

Chemokine receptors, such as CXCR2, are G-protein coupled receptors (GPCRs) typically linked to pertussis toxin (PTX) sensitive Gi proteins (Bajetto et al., Front Neuroendocrinol. 22:147-184 (2001)). The CXCR's N-terminus domain is thought to be important for determining ligand binding specificity. CXCR2 binds CXCL1, a soluble secreted chemoattractive cytokine of the ELR positive family of CXC chemokines, and their interaction activates intracellular signals that modulate processes such as proliferation, differentiation, and migration (Bajetto et al., Front Neuroendocrinol. 22:147-184 (2001)); Miller, Prog. Neurobiol. 67:451-467 (2002); Tsai et al., Cell 110:373-383 (2002)). Changes in migration are thought to take effect by the modulation of actin-dependent cellular processes and adhesion molecule expression (Ransohoff J. Neuroimmunol. 98:57-68 (1999)). Upon binding its ligands in the vasculature, CXCR2 modulates adhesion molecule expression on the surface of some leukocytes to allow their rolling, adhesion, arrest, and diapedesis for tissue infiltration (Smith et al., Am J Physiol Heart Circ Physiol 289:H1976-84 (2005)). This is commonly observed in monocytes and neutrophils expressing the chemokine receptor CXCR2. Once in the tissue, these cells are typically further guided by chemokines to inflammatory sites by chemotaxis.

Although each chemokine receptor usually binds a single class of chemokines, they can bind several members of the same class with high affinity (Horuk, Cytokine Growth Factor Rev. 12:313-335 (2001); Horuk et al., J. Immunol. 158:2882-2890 (1997)). In addition, one chemokine can bind several different chemokine receptors (Horuk, Cytokine Growth Factor Rev. 12:313-335 (2001)). CXCR2, for example, can bind CXCL1, CXCL2, CXCL3, CXCL5, CXCL6, CXCL7, and CXCL8 (Miller and Meucci, Trends Neurosci. 22:471-479 (1999)).

CXC chemokines and their receptors are now known to be expressed in the vertebrate CNS (Tran and Miller, Nat. Rev. Neurosci. 4:444-455 (2003)). Chemokines and chemokine receptors were initially characterized as activators and chemoattractants for leukocytes and other cells of the immune system. (Fernandez and Lolis, Annu. Rev. Pharmacol. Toxicol. 42:469-499 (2002)). For example, the CXCL1 chemokine receptor, CXCR2, is expressed on subsets of neurons (Cho and Miller, J. Neurovirol. 8:573-84 (2002); Coughlan et al., Neuroscience 97:591-600 (2000)), astrocytes (Flynn et al., J. Neuroimmunol. 136:84-93 (2003)), microglia, and oligodendrocyte progenitor cells (OPCs) (Nguyen and Stangel, Dev. Brain Res. 128:77-81 (2001)).

In the CNS, expression of functional chemokine receptors has been demonstrated in neurons (Bajetto et al., 2001); astrocytes (Filipovic et al., Dev. Neurosci. 25:279-290 (2003); Flynn et al., J. Neuroimmunol. 136:84-93 (2003);), oligodendrocytes (Filipovic et al., Dev. Neurosci. 25:279-290 (2003); Kadi et al., J. Neuroimmunol. 174:133-146 (2006); Tsai et al., Cell 110:373-383 (2002); Wu et al., J. Neurosci. 20:2609-2617 (2000)), and microglia (Filipovic et al., Dev. Neurosci. 25:279-290 (2003); Flynn et al., J. Neuroimmunol. 136:84-93 (2003);) under physiological conditions. Chemokine signaling can regulate migration, proliferation, differentiation, and activation of cells, in both the immune system and the CNS, in development and after pathology (Kadi et al., J. Neuroimmunol. 174:133-146 (2006)). The ancestral phylogenic role of the CXC chemokine family, however, has been reported to reside in the nervous, rather than the immune system (Huising et al., Trends Immunol. 24:307-313 (2003)).

Furthermore, the roles of some chemokines and their receptors such as CXCR2 (binds CXCL1-3, 5-8) in normal development and in pathological processes in the CNS are starting to be more widely studied (Coughlan et al., Neuroscience 97:591-600 (2000) Flynn et al., J. Neuroimmunol. 136:84-93 (2003); Omari et al., Glia 53(1):24-31 (2005)), CXCR3 (binds CXCL9-11) (Coughlan et al., Neuroscience 97:591-600 (2000); Flynn et al., J. Neuroimmunol. 136:84-93 (2003); Omari et al., Brain 128)), CCR3 (binds CCL5, 7, 8, 11, 13, 15, 24, 26, 28) (Coughlan et al., Neuroscience 97:591-600 (2000); Flynn et al., J. Neuroimmunol. 136:84-93 (2003); Xia and Hyman, J. Neurovirol. 5:32-41 (1999)) and CXCR4 (binds CXCL12) (Flynn et al., J. Neuroimmunol. 136:84-93 (2003) Lieberam et al., Neuron 47:667-679 (2005)). For example, astrocytes and microglia express CXCR3 (Miller and Meucci, Trends Neurosci. 22:471-479 (1999)). CXCL9 and CXCL10, the ligands for CXCR3 (Miller and Meucci, Trends Neurosci. 22:471-479 (1999)), are upregulated after brain damage, inducing the chemotaxis of these cells (Biber et al., Neuroscience 112:487-497 (2002)). CXCR4 signaling is important for retinal growth cone guidance (Chalasani et al., J. Neurosci. 23:1360-1371 (2003)), mammalian motor axon pathfinding (Lieberam et al., Neuron 47:667-679 (2005)), sensory neuron progenitor migration (Belmadani et al., J. Neurosci. 25:3995-4003 (2005)), and limb innervation (Odemis et al., Mol. Cell. Neurosci. 30:494-505 (2005)). Activation of CXCR4 by its ligand, SDF-1/CXCL12, can guide neural precursor cells to injury sites within the CNS (Imitola et al., Proc. Natl. Acad. Sci. USA 101:18117-18122 (2004)). These neural precursors can give rise to neurons, astrocytes, and oligodendrocytes (Ni et al., Dev. Brain Res. 152:159-169 (2004)).

In addition, CXCL12 is upregulated in astrocytes and blood vessels around areas of demyelination in MS, where it may serve to modulate immune cell infiltration through the blood brain barrier (Krumbholz et al., Brain 129:200-211 (2006)). CXCR2 is expressed in projection neurons in the brain and spinal cord (Horuk et al., J. Immunol. 158:2882-2890 (1997)) and its activation has been shown to enhance survival of hippocampal neurons. It is also expressed around neuritic plaques in colocalization with amyloid β precursor protein in Alzheimer's disease (Xia and Hyman, J. Neurovirol. 5:32-41 (1999)) and its expression, along with that of its ligand CXCL1, is upregulated following experimental closed head injury in rats (Valles et al., Neurobiol. Dis. 22:312-322 (2006)). Finally, CXCL1 has been shown to modulate oligodendrocyte responses during the development of the CNS (Robinson et al., J. Neurosci. 18:10457-10463 (1998); Tsai et al., Cell 110:373-383 (2002)). Although the roles of chemokines in nervous system development and pathology have been widely investigated, relatively little is still known in comparison to their roles in the immune system, raising the necessity for investigation.

OPCs express the chemokine receptor CXCR2 and can therefore bind and respond to CXCL1. During early postnatal development in mice, CXCL1 can enhance the PDGF induced proliferation and decrease the migration of OPCs (Robinson et al., J. Neurosci. 18:10457-10463 (1998); Tsai et al., Cell 110:373-383 (2002)). CXCR2 signaling in response to astrocyte-secreted CXCL1 helps position OPCs in the presumptive white matter. It also locally modulates responses to PDGF, enhancing the proliferative response of OPCs so that proper oligodendrocyte numbers to successfully myelinate developing axons are achieved (Tsai et al., Cell 110:373-383 (2002)).

Despite a wealth of studies done on CNS and peripheral nervous system, there remains a considerable need for developing methods for treating neuropathies including demyelination. The present invention is directed to methods for treating neuropathy by targeting chemokine-mediated signaling.

### SUMMARY OF THE INVENTION

The present invention provides methods directed to treating a neuropathy in a subject comprising administering an effective amount of a bioactive agent to modulate CXC chemokine signaling. The subject methods may reduce central nervous system (CNS) immune infiltration enhance neural cell migration, proliferation, and/or differentiation.

In one aspect of the invention, the present invention provides a method of ameliorating a neuropathy comprising administering to a subject in need thereof a therapeutically effective amount of a bioactive agent that selectively inhibits CXCR1 and/or CXCR2-mediated signaling relative to other CXC receptors as ascertained in a cell-based assay. In some embodiments, neuropathy is a demylination condition including but not limited to multiple sclerosis.

In yet another aspect, the present invention provides a method of promoting glial cell migration comprising contacting a glial cell with a bioactive agent that inhibits CXCR-mediated signaling in the glial cell, wherein said migration is increased as compared to a glial cell not contacted with the bioactive agent. Further provided is a method of promoting remyelination comprising administering to a subject exhibiting a demyelinating lesion with a bioactive agent, wherein the bioactive agent is effective in reducing gliosis through CXCR-mediated signaling, thereby promoting remyelination in the subject. In some embodiments, the CXCR-mediated signaling is via CXCR1 and/or CXCR2.

Also provided in the present invention is a method of promoting glial cell proliferation and/or differentiation comprising contacting a glial cell with a bioactive agent that selectively inhibits CXCR1 and/or CXCR2-mediated signaling relative to other CXC receptors as ascertained in a cell-based assay, wherein the proliferation and/or differentiation is increased as compared to a glial cell not contacted with the bioactive agent. In another aspect the present invention provides, a method of ameliorating gliosis comprising administering to a subject in need thereof a therapeutically effective amount of a bioactive agent that selectively inhibits CXCR1 and/or CXCR2 mediated signaling relative to other CXC receptors as ascertained in a cell-based assay. In some embodiments, the other CXC receptors are CXCR3 or CXCR4.

In another aspect of the present invention, the bioactive agent directly binds CXCR1 and/or CXCR2. In some embodiments, the bioactive agent inactivates CXCL1, CXCL2, CXCL3, CXCL5, CXCL7, or CXCL8. In yet other embodiments, the bioactive agent reduces CXCR1 and/or CXCR2 activity.

In some embodiments, the bioactive agent includes without limitation a peptide, polypeptide, antibody, antisense molecule, siRNA, small molecule or peptidomimetic. In various embodiments, the bioactive agent is selected from the group of compounds in FIGS. 10A, 10B, 10C and 11.

In yet another aspect, the bioactive agent may reduce expression of GFAP, vimentin, heparan sulphate proteoglycan (HSPG), dermatan sulphate proteoglycan (DSPG), keratan sulphate proteoglycan (KSPG), or chondroitin sulphate proteoglycan (CSPG).

Further, the glial cell of the present invention may be selected from a group consisting of oligodendrocyte, oligodendrocyte progenitor, Schwann, astrocytes, microglial and a combination thereof.

### BRIEF DESCRIPTION OF THE DRAWINGS

FIG. 1. Regional differences in oligodendrocyte lineage cell density in Cxcr2^{-/-}:PLP/DM20-EGFP⁺ mice: (A) Overall Cxcr2^{-/-} mice display decreased intensity of PLP/DM20-EGFP expression in the CNS when compared to WT sex-matched littermates. This decreased intensity did not correlate with a reduction in number of oligodendrocyte lineage cells, which varied between CNS regions. (B,C) The spinal cord and corpus callosum contained an increased EGFP⁺ cell density while the cortex and anterior commissure had decreased EGFP⁺ cell density. No significant differences were seen in the hippocampus (HC) or cerebellum (CB). (D) Matching images of PLP/DM20-EGFP⁺ cells from WT (top) and Cxcr2^{-/-} (bottom) dorsal columns of the spinal cord, the corpus callosum, and the anterior commissure. Note the higher number of EGFP⁺ cells in the Cxcr2^{-/-} dorsal spinal cord and lower number in the anterior commissure. Data is plotted as means+/−2*SEM from at least 3 Cxcr2^{-/-} and 3 Cxcr2^{-/-} adult (≧6 weeks) mice. p values were determined by one-way ANOVA, where *=p<0.05, **=p<0.01, and ***=p<0.001. Bars=1 mm in A and 100 μm in D.

FIG. 2. Increased density and process arborization of NG2⁺ cells in Cxcr2^{-/-} mice: (A) The spinal cords of Cxcr2^{-/-} mice have increased numbers of NG2⁺ cells that are more concentrated at the pial surface than in WT. (B) NG2⁺ cells in Cxcr2^{-/-} animals have more processes with stronger expression of NG2 than in WT mice. (C) Cxcr2^{-/-} spinal cords have a mean increase of 22% in the density of NG2⁺ cells. Data is plotted as means+/−SD from at least 3 Cxcr2^{-/-} and 3 Cxcr2^{+/+} adult (≧6 weeks) mice. p value was determined by one-way ANOVA, where*=p<0.05. Bars=250 μm in A and 50 μm in B.

FIG. 3. Reduced growth and strain related phenotypic alterations in BALB/c and BALB/c:C57BL/6 Cxcr2^{-/-} mice: (A) Cxcr2^{-/-} mice in the mixed BALB/c:C57BL/6 background exhibited decreased growth compared to WT littermates. They also developed particular facial features, with a more rounded appearance to the face and shortened nose. (B) The growth reduction was also seen in the BALB/c Cxcr2^{-/-} mice, but to a much lesser extent. (C) The brains from Cxcr2^{-/-} mice were smaller than WT sex-matched littermate controls (brains from mice in mix background illustrated). This reduction was proportional to the decrease in overall body size. Weight data is plotted as means+/−SD from (A) 60 Cxcr2^{-/-} and 57 Cxcr2^{+/+} mice grouped into different age ranges from birth, and from (B) approximately 12 adults (≧6 weeks) from each genotype in each background. p values were determined by one-way ANOVA, where *=p<0.05 and ***=p<0.001; NS=not significant.

FIG. 4. Persistent white matter reduction in the spinal cord of Cxcr2^{-/-} mice: (A,B) Comparison of the relative area of white matter to total spinal cord area demonstrates a reduction in white matter area in the Cxcr2^{-/-} mice. Inset in A shows half cross sections of spinal cords from p7 Cxcr2^{-/-} and WT mice, illustrating the reduction in white matter. (A) This difference is reduced during development but remains significant in adulthood (≧6 weeks). (B) An overall mean reduction of 14% was seen in white matter area in Cxcr2^{-/-} mice. Data for individual ages is plotted as means+/−SD and the cumulative data as means+/−2*SEM. The data collected is from 10 Cxcr2^{-/-} and 10 Cxcr2^{+/+} mice. p values were determined by one-way ANOVA, where *=p<0.05, **=p<0.01, and ***=p<0.001. Bar=100 μm.

FIG. 5. Persistent hypomyelination in the spinal cord of Cxcr2^{-/-} mice: (A-E) Electron micrographs of similarly sized axons from WT (A,D) and Cxcr2^{-/-} (B,C,E) mice show that the thickness of myelin is reduced around axons of all sizes in Cxcr2^{-/-} mice. (F) To quantify the differences, and determine their significance, 150-350 random axons were measured from matching Cxcr2^{-/-} and WT sex-matched littermate mouse spinal cords at different ages, and their myelin thickness to axon perimeter ratio calculated. The results are plotted as means+/−2*SEM. p values were determined by one-way ANOVA, where *=p<0.05,**=p<0.01, and ***=p<0.001. Scale bar in A-E=200 nm.

FIG. 6. Decreased central conduction velocity in Cxcr2^{-/-} mice: The lumbar spinal cord of 24 month old Cxcr2^{-/-} and Cxcr2^{+/+} mice in both BALB/c:C57BL/6 and pure BALB/c backgrounds was stimulated with subdermal electrodes, and recordings of spinally elicited evoked potentials were made rostrally. A significant reduction in conduction velocity was seen in Cxcr2^{-/-} mice regardless of their genetic background, indicating that these changes are independent of the growth phenotype. The results are plotted as means+/−SD and p values were determined by one-way ANOVA, where *=p<0.05 and ***=p<0.001.

FIG. 7. Normal myelin compaction and periodicity, and paranodal and nodal structures in Cxcr2^{-/-} mice: (A) Myelin from Cxcr2^{-/-} mice, although thinner, had normal compaction and periodicity. The box represents the super imposition of Cxcr2^{-/-} myelin from a similarly sized axon as in the WT image under it. (B) Paranodal loops and transverse bands are well developed and oriented in Cxcr2^{-/-} mice and (C) no striking differences were apparent in the structure of the nodes of Ranvier and paranodal regions. Scale bar in A-C=200 nm.

FIG. 8. Illustrates a schematic of oligodendorcytes myelinating CNS axons.

FIG. 9. Defective oligodendrocyte maturation in Cxcr2^{-/-} spinal cord derived mixed cultures: Mixed spinal cord cultures from p8 Cxcr2^{-/-} and WT littermates were generated and stained for (A-B) O4, a marker of pre-oligodendrocytes, and (C-D) O1, a marker of newly differentiated oligodendrocytes, 48 hours after plating. When the number of positively stained cells relative to total number of cells was quantified in cultures from Cxcr2^{-/-} and WT mice, Cxcr2^{-/-} cultures exhibited a significant reduction (˜60%) in the generation of O1⁺ cells (F). There was no significant difference, however, in the generation of O4⁺ cells between Cxcr2^{-/-} and WT mice (E). The results are plotted as means+/−SD. p value was determined by one-way ANOVA, where *=p<0.05. Scale bar=50 μm.

FIGS. 10A-C. FIGS. 10A-B illustrates various CXCR1/CXCR2 antagonists, while FIG. 10C illustrates various CXCR1/CXCR2 allosteric inhibitors.

FIG. 11 Illustrates the structural formula for repertaxin (R(−)-2-(4-isobutylphenyl)propionyl methansulphonamide). As shown, repertaxin is salified with L-lysine.

FIG. 12. Reduced expression of myelin basic protein (MBP), proteolipid protein (PLP), and glial fibrillary acidic protein (GFAP) in the CNS of Cxcr2^{-/-} mice: (A) Decreased expression of MBP was seen on Western blots of brains from 2-4 week old Cxcr2^{-/-} mice regardless of sex or age. The expression of PLP was also reduced in 4 week old Cxcr2^{-/-} mice, consistent with hypomyelination. Detection of GFAP was similarly decreased in Western blots from 4 week old Cxcr2^{-/-} mice. No difference was apparent between Cxcr2^{+/-} and Cxcr2^{+/+} mice in the levels of these proteins. (B,C) Immunohistochemistry on spinal cords (B) and brains (C) of Cxcr2^{+/+}:PLP/DM20-EGFP⁺ and Cxcr2^{-/-}:PLP/DM20-EGFP⁺ mice was used to confirm the reduction of GFAP observed by Western blot. β-actin was used as a marker of protein loading in all Western blots. Scale bar=100 μm.

FIG. 13. CXCR2 inhibition appears to induce oligodendrocyte survival after toxin induced demyelination: The expression of MBP and PLP are compared between Cxcr2^{+/+} and Cxcr2^{-/-} sex matched littermates before (A-A″ and B-B″) and after 4 weeks (C-C″ and D-D″) of cuprizone treatment. The expression of PLP promoter driven EGFP is used in these mice as a marker of cells of the oligodendrocyte lineage. In the non treated group, Cxcr2^{-/-} mice had decreased expression of PLP (B′<A′) when compared to their Cxcr2^{+/+} littermates (A′>B′). After 4 week of cuprizone exposure, the Cxcr2^{-/-} mice did not exhibit any alterations in the expression of PLP (B′=D′), while Cxcr2^{+/+} mice, exhibited a dramatic decrease in the expression of PLP (A′>>>C′). The cellularity in the corpus callosum (arrows pointing to dotted area) was also dramatically reduced in Cxcr2^{+/+} mice when compared to levels before treatment (C″<A″) and to levels in their Cxcr2^{-/-} sex matched littermates after 4 weeks of Cuprizone treatment (C″<D″). This indicates that there is loss of oligodendrocytes in wild-type mice and protection from death or enhanced survival of oligodendrocytes in Cxcr2^{-/-} mice.

FIG. 14. Demyelination in the corpus callosum of Cxcr2^{+/+}, but not of Cxcr2^{-/-} mice after 4 weeks of cuprizone treatment: MBP immunohistochemistry of matching areas of the brains of sex matched Cxcr2^{+/+} and Cxcr2^{-/-} littermate mice 4 weeks after the initiation of cuprizone treatment revealed areas of myelin loss in the Cxcr2^{+/+} (A, arrow)) but not in the Cxcr2^{-/-} mice (B). Areas devoid of MBP staining in the brains of Cxcr2^{+/+} mice (A) correlated with areas of decreased PLP expression (A′) and decreased cellularity (A″) as indicated by the arrows in column A-A′″. These changes where not seen in Cxcr2^{-/-} mice (B-B″).

FIG. 15. Decreased lesion load in the corpus callosum of Cxcr2^{-/-} mice when compared to Cxcr2^{+/+} mice after 7 weeks of cuprizone treatment: Areas of extensive myelin destruction (arrows) can be observed by the lack of expression of both MBP and PLP in the corpus callosum of Cxcr2^{+/+} mice (A-A″, C-C″), but not of their Cxcr2^{-/-} sex matched littermates (B-B″). In MBP stains of tissue from Cxcr2^{+/+} mice, certain areas expressing very low levels of MBP (dotted, C), correlated with high density of PLP-EGFP⁺ cells (dotted, C′) indicating that these cells may be attempting to remyelinate demyelinated foci (dotted, C″).

FIG. 16. Astrogliosis in the corpus callosum of Cxcr2^{+/+}, but not of Cxcr2^{-/-} mice after 4 weeks of cuprizone treatment: Analysis of CNS tissue from Cxcr2^{-/-} and Cxcr2^{+/+} mice had previously revealed a reduction in the expression of GFAP in Cxcr2^{-/-} mice (B<A) indicating that CXCR2 could modulate astrocyte physiology. Consistent with this observation, GFAP upregulation was not seen in Cxcr2^{-/-} mice after exposure to cuprizone for 4 weeks (D), a time point when significant GFAP upregulation is observed in Cxcr2^{+/+} mice (C). This difference was most striking within the corpus callosum (dotted area).

FIG. 17. Astrogliosis in the brain of Cxcr2^{+/+}, but not of Cxcr2^{-/-} mice after 6 weeks of cuprizone treatment: Despite the fact that Cxcr2^{+/+} mice still exhibit prominent astrogliosis in the corpus callosum (dotted areas) 6 weeks into cuprizone treatment (B), GFAP staining of Cxcr2^{-/-} tissue (A,C) appears indistinguishable from levels before treatment. The enhancement of GFAP expression in the Cxcr2^{+/+} mice (B) at week 6 of treatment, correlates with decreased PLP expression (B′), while no changes in these parameters were evident in the Cxcr2^{-/-} mice (A-A′,C-C′). The cellularity of the corpus callosum in Cxcr2^{+/+} mice at 6 weeks (B″, circle) is higher than it was before treatment, and correlates with increased GFAP expression (B,B′″, circle) and decreased PLP expression (B′, circle), indicating the infiltration of astrocytes into the lesion were oligodendrocytes used to reside.

FIG. 18. Alteration in microglial responses to cuprizone treatment in Cxcr2^{-/-} mice: Non-injured Cxcr2^{-/-} mice had increased IBA-1 expression (B) when compared to their Cxcr2^{+/+} littermate controls (A). Chemokine treatment for 4 and 7 weeks induced significant activation of these IBA-1 positive microglia in Cxcr2^{+/+} mice (C, E, respectively). Microglial activation was not observed at 4 weeks, and was minimal at 7 weeks of treatment in Cxcr2^{-/-} mice (D, F, respectively). By week 7, the density of IBA-1⁺ cells within the corpus callosum of Cxcr2^{+/+} mice (E) was very high, while it was close to normal in Cxcr2^{-/-} mice (F).

FIG. 19. Alterations in the number and shape of IBA-1⁺ cells in response to cuprizone treatment in Cxcr2^{+/+} and Cxcr2^{-/-} mice: The differences observed in IBA-1⁺ cells after cuprizone treatment of Cxcr2^{+/+} and Cxcr2^{-/-} mice appear to be due to changes in both the number and the activation state or structure of these cells. IBA-1⁺ cells from the Cxcr2^{+/+} mice (A,C) generally appeared fuller and in higher density than those from Cxcr2^{-/-} mice. IBA-1⁺ cells in the Cxcr2^{-/-} mice (B,D), instead of looking plump, were characterized by having multiple long and thin branches typical of resting or quiescent microglia.

FIG. 20. Expression of CXCR2 on peripheral nervous system Schwann cells: Immunohistochemistry for CXCR2 on sciatic nerves derived from Cxcr2^{-/-}:PLP/DM20-EGFP⁺ and Cxcr2^{+/+}:PLP/DM20-EGFP⁺ mice revealed expression of CXCR2 on a subset of PLP-EGFP⁺ Schwann cells.

FIG. 21. Hypomyelination of sciatic nerve axons in Cxcr2^{-/-} mice: Cxcr2^{-/-} mice appear to have hypomyelination in the peripheral nervous system (PNS) in addition to that observed in the CNS. Electron micrographs of similarly sized axons from the sciatic nerves of Cxcr2^{+/+} and Cxcr2^{-/-} mice are shown. The thickness of myelin is reduced around axons of all sizes, but appears to be more reduced around large axons. To quantify the differences, and determine their significance, ˜100 random axons from the sciatic nerves of one Cxcr2^{-/-} and two wild-type sex-matched littermate mice were measured and the myelin thickness to axon perimeter ratio calculated. The results are plotted as means+/−2*SEM. p=0.0003.

FIG. 22. Possible contribution of the peripheral nervous system to decline in conduction of nervous impulses in Cxcr2^{-/-} mice: Cxcr2^{-/-} animals demonstrate impairment in central conduction of spinally elicited evoked potentials and somatosensory evoked potentials when compared to WT littermates. The lumbar spinal cord (for CNS) or the tibial nerve (PNS+CNS) of 2-4 month old Cxcr2^{-/-} and Cxcr2^{+/+} mice in both BALB/c:C57BL/6 and pure BALB/c backgrounds were stimulated with subdermal electrodes and recordings made rostrally. A significant reduction in conduction velocity was seen in Cxcr2^{-/-} mice (15-30%) regardless of their genetic background, indicating that these changes are independent of the growth phenotype observed in mice of the mixed background. When the percent in conduction velocity reduction was compared between experiments including a peripheral component, or those restricted to the CNS, it appeared that a peripheral component may be contributing to the decrease in conduction velocity in BALB/c mice. This is consistent with the hypomyelination illustrated in FIG. 21.

FIG. 23. Origin and dispersal of oligodendrocyte precursors in the spinal cord: Spinal cord oligodendrocytes arise in the ventricular zone (VZ) and, through a combination of local chemorepellant and distant chemoattractive cues, migrate to presumptive white matter where they stop migrating and proliferate in response to mitogenic signals (left). Once the proliferative phase is complete these cells will respond to local differentiative cues and eventually mature into myelinating oligodendrocytes (right). (VZ=ventricular zone; FP=floor plate, NTC=notochord)

FIG. 24. Oligodendrocyte and astrocyte cell lineage: Initially developing from stem cells, as do neurons and type I astrocytes, oligodendrocyte precursor cells (OPCs), which express A2B5, NG2, and platelet derived growth factor receptor alpha (PDGFRα), typically have the capacity in vitro to constitutively differentiate into pre-oligodendrocytes or be induced to produce type II astrocytes by bone morphogenetic protein 4 (BMP4). In their differentiation, pre-oligodendrocytes generally acquire the expression of sulfatide, which can be identified by the monoclonal antibody (mAb) O4. Subsequently, they typically acquire the expression of galactocerebrosides and are classified as immature oligodendrocytes. Immature oligodendrocytes can be labeled with mAb O1 and typically depend on specific cues/signals for their survival, in the absence of which they undergo apoptosis. Those cells which do not undergo programmed cell death can mature further. Immature and non-myelinating and myelinating mature oligodendrocytes typically no longer respond to the mitogen PDGF. The most mature forms, capable of producing myelin, generally acquire the expression of myelin specific proteins such as myelin basic protein (MBP) and myelin oligodendrocyte glycoprotein (MOG). Oligodendrocyte lineage cells typically express DM20 and/or PLP throughout all stages of their development. (Baumann and Pham-Dinh, Physiol. Rev. 81:871-927 (2001); Miller, Prog. Neurobiol. 67:451-467 (2002); Polito and Reynolds, J. Anat. 207:707-716 (2005))

FIG. 25. Oligodendrocytes, myelin, and nerve conduction: Myelin, the fatty insulation from the oligodendrocyte membrane which wraps around axons, aids in the rapid propagation of action potentials in the CNS. In unmyelinated axons (top) there is a continuous wave of depolarization as action potentials spread sequentially along the bare axonal membrane. In myelinated axons (bottom) the flow of energy is not continuous but rather “jumps” from one node to the next, where sodium channels accumulate to regenerate action potentials. Depolarization becomes discontinuous in a process termed salutatory conduction, thus making conduction of nerve impulses faster than in non-myelinated fibers. (OD=Oligodendrocyte, N=Neuron)

FIG. 26. Sodium channel rearrangement and alterations in conduction velocity following demyelination: Conduction block can occur in demyelinated axons because the membrane of the axon underlying the internode has a very low concentration of sodium channels, which are essential to regenerate action potentials. When this membrane is exposed, action potentials cannot spread through the area devoid of myelin. The upregulation of sodium channels (brown dots) in the membrane that used to be covered by myelin subsequently occurs in an attempt to restore proper conduction. (OD=Oligodendrocytes)

FIG. 27. Types of multiple sclerosis: The demyelinating disease multiple sclerosis is classified into different types according to the presence or absence of remissions and the accumulation of disabling symptoms. In benign remitting (BR) MS (˜10%), relapses are present, but compensatory mechanisms appear to be sufficient to restore baseline function to the levels before the relapse. In relapsing remitting (RR) MS (˜50%), recovery after a relapse is not complete and a degree of disability is maintained. As more relapses occur the symptoms worsen and disability increases. Secondary progressive (SP) MS (˜30%) starts out as RRMS but at some point no clear remissions occur and the patient's health continues to deteriorate. In primary progressive (PP) MS (˜10%), there are no remissions but rather a continuous accumulation of disabling symptoms. (Joy and Johnston, Editors. Multiple Sclerosis: Current Status and Strategies for the Future (2001))

FIG. 28. The G-protein coupled chemokine receptor CXCR2, its ligands, and its functions: The chemokine receptor CXCR2 is a member of the G-protein coupled receptor (GPCR) family. CXCR2 can bind CXCL1, a soluble secreted chemoattractive cytokine of the ELR positive family of CXC chemokines. Their interaction activates intracellular signals that modulate processes such as proliferation, differentiation, and migration. GPCRs are 7 transmembrane spanning receptors and their intracellular carboxy terminus and cytoplasmic loops bind heterotrimeric G-protein complexes composed of three subunits (α,β,γ). Upon activation, the α and βγ portions dissociate and further activate intracellular effectors that act as second messengers. Some of their targets are phospholipase-C (PLC), whose activation leads to phosphatidylinositol (PI) production and intracellular calcium increase, and PI3 kinase (PI3K) whose products can organize the actin based cytoskeleton for polarized migration during chemotaxis. The activation of the heterotrimeric G-protein complex can be inhibited by arresting, regulatory proteins that bind the cytoplasmic part of GPCRs and prevent their interaction with the heterotrimeric G-protein complex. Arrestin binding also aids in receptor internalization and recycling, thus modulating receptor signaling.

FIG. 29. CXCL1/CXCR2 effects on oligodendrocyte precursor cells (OPCs): Oligodendrocyte precursor cells (OPCs) are directed to the presumptive white matter by a combination of chemorepellant and chemoattractive cues. At the presumptive white matter, it is thought that they encounter the chemokine CXCL1, secreted locally by astrocytes. Binding of CXCL1 to CXCR2 on the surface of OPCs induces these cells to stop migrating and become more responsive to the mitogen PDGF (platelet derive growth factor). Proliferation is needed to ensure that adequate numbers of oligodendrocytes develop to meet the myelin demands of locally developing axons.

FIG. 30. Cxcr2 genotyping by polymerase chain reaction (PCR): Mouse DNA samples isolated from tail clips are loaded into the wells of a 1.2% agarose gel next to a standard DNA ladder with fragments of known size. The chamber containing the gel produces an electric current. The negatively charged DNA is repelled by the negative charges at the top of the chamber and attracted by the positive charges at the bottom. As the DNA migrates, larger sized DNA fragments run slower and migrate less far. The Cxcr2^{-/-} mice have a deletion in the Cxcr2 gene and thus their DNA is shorter (280 base pairs (bp) in contrast to 360 bp in the wild-type) and runs further in the gel.

FIG. 31. White matter to spinal cord area measurements: The edge of the spinal cord as well as the interface between gray (center) and white matter (green edge) were traced to obtain area measurements. The area of the white matter was calculated by subtracting the area of the gray matter from the total spinal cord area and with these values a ratio of white matter to total spinal cord area was calculated.

FIG. 32. Myelin thickness to axon perimeter measurements: The picture illustrates and axon in cross section photographed using an electron microscope with superimposed tracings of the axon perimeter in yellow, and three separate measurements of myelin thickness in black (lines X,Y,Z). An average myelin thickness was calculated and divided by the axon perimeter to determine their ratio.

FIG. 33. Purified spinal cord astrocytes express CXCL1 and CXCR2 mRNA: RT PCR was performed on RNA extracted from purified astrocytes derived from spinal cord. Results indicate CXCL1 and CXCR2 are present in astrocytes.

FIG. 34. CXCL1 effects on astrocytes and induction of glial scars. Addition of CXCL1 (0.5 ng/ml) to purified astrocyte cultures induces CSPG expression and changes morphology. Cells were treated with CXCL1 (0.5 ng/ml) for 3 days and assayed for CSPG deposition onto substrate by immunocytochemistry and released into medium. By dot blot (inset) CXCL1 increased CSPG (antibody to CSPG). GFAP (red), CSPG (green).

FIG. 35. CXCL1 and CXCR2 are induced in demyelinating lesions. Immunohistochemistry analyzing CXCR1 and CXCR23 days after LPC lesions. CXCL protein is upregulated within the lesion as indicated by DAB staining. CXCR2 is colocalized with GFAP+ cells in the lesion but not outside.

FIG. 36. Neutralization of CXCR2 decreases demyelinating LPC lesions. Local injection of neutralizing anti-CXCR2 antibody into a LPC induced lesion results in a reduction in lesion size and substantial morphological recovery. Luxol Fast Blue staining (top panels) and 3 dimensional reconstruction (bottom panels) of 10 day LPC lesions treated with isotype control antibody (left panels) or neutralizing antibody against. CXCR2 (right panels). Reconstruction indicates a decrease in lesion volume by 96% in animals with blockage of CXCR2 (graph).

FIG. 37. Neutralization of CXCR2 decreases GFAP immunoreactivity and ED1+reactivity in outlying regions of the lesion.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

### General Techniques:

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### DEFINITIONS

As used in the specification and claims, the singular form “a”, “an” and “the” include plural references unless the context clearly dictates otherwise. For example, the term “a cell” includes a plurality of cells, including mixtures thereof.

The term “control” is an alternative subject, cell or sample used in an experiment for comparison purpose. Furthermore, a “control” can also represent the same subject, cell or sample in an experiment for comparison of different time points.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectedly referred to as “gene product.” If the polynucleotide is derived from genomic DNA, expression may include splicing of the mRNA in a eukaryotic cell.

The terms “delivery” and “administration” are used interchangeably herein to mean an agent enters a subject, tissue or cell. The terms used throughout the disclosure herein also include grammatical variances of a particular term. For example, “delivery” includes “delivering”, “delivered”, “deliver”, etc. Various methods of delivery or administration of bioactive agents are known in the art. For example, one or more agents described herein can be delivered parenterally, orally, intraperitoneally, intravenously, intra-arterially, transdermally, intramuscularly, liposomally, via local delivery by catheter or stent, subcutaneously, intra-adiposally, or intrathecally. In addition, depending on the characteristics of the agent, an agent can be delivered via plasmid vectors, viral vectors or non-viral vector systems, including liposome formulations and minicells.

The term “differentially expressed” as applied to nucleotide sequence or polypeptide sequence in a subject, refers to over-expression or under-expression of that sequence when compared to that detected in a control. Under-expression also encompasses absence of expression of a particular sequence as evidenced by the absence of detectable expression in a test subject when compared to a control.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

A “subject,” “individual” or “patient” is used interchangeably herein, which refers to a vertebrate, preferably a mammal, more preferably a human. Mammals include, but are not limited to mice, rats, dogs, pigs, monkeys (simians) humans, farm animals, sport animals, and pets. Tissues, cells and their progeny of a biological entity obtained in vivo or cultured in vitro are also encompassed.

“Signal transduction” is a process during which stimulatory or inhibitory signals are transmitted into and within a cell to elicit an intracellular response. A “modulator of a signal transduction pathway” refers to a substance which modulates the activity of one or more cellular proteins mapped to the same specific signal transduction pathway. A modulator may augment or suppress the activity and/or expression level or pattern of a signaling molecule. In the context of CXC chemokine signaling, modulation of the signaling pathway encompasses changes in expression level or pattern of a CXC chemokine or its corresponding receptor, as well as that of any downstream or upstream signaling molecules in the pathway(s) in which the CXC chemokine or its corresponding receptor is a member.

As used in herein “cell” is used in its usual biological sense, and does not refer to an entire multicellular organism. The cell can, for example, be in vitro, e.g., in cell culture, or present in a multicellular organism, including, e.g., birds, plants and mammals such as humans, cows, sheep, apes, monkeys, swine, dogs, cats, mice or rats.

As used herein, “treatment” or “treating,” or “ameliorating” are used interchangeably herein. These terms refers to an approach for obtaining beneficial or desired results including and preferably clinical results. For purposes of this invention, beneficial or desired clinical results include, but are not limited to, one or more of the following: shrinking the size of demyelinating lesions (in the context of demyelination disorder, for example), promoting OPC proliferation and growth or migration to lesion sites, promoting differentiation of oligodendrocytes, delaying the onset of a neuropathy, delaying the development of demyelinating disorder, decreasing symptoms resulting from a neuropathy, increasing the quality of life of those suffering from the disease, decreasing the dose of other medications required to treat the disease, enhancing the effect of another medication such as via targeting and/or internalization, delaying the progression of the disease, and/or prolonging survival of individuals. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The terms “agent”, “biologically active agent”, “bioactive agent”, “bioactive compound” or “biologically active compound” are used interchangeably and also encompass plural references in the context stated. Such compounds utilized in one or more combinatorial treatment methods of the invention described herein, include but are not limited to a biological or chemical compound such as a simple or complex organic or inorganic molecule, peptide, peptide mimetic, protein (e.g. antibody), nuclei acid molecules including DNA, RNA and analogs thereof, carbohydrate-containing molecule, phospholipids, liposome, small interfering RNA, a polynucleotide (e.g. anti-sense), or a combination external guided sequence (EGS).

The term “antagonist” as used herein refers to a molecule having the ability to inhibit a biological function of a target polypeptide. Accordingly, the term “antagonist” is defined in the context of the biological role of the target polypeptide. While certain antagonists herein specifically interact with (e.g. bind to) the target, molecules that inhibit a biological activity of the target polypeptide by interacting with other members of the signal transduction pathway of which the target polypeptide is a member are also specifically included within this definition. One biological activity inhibited by an antagonist is associated with increasing proliferation of OPC, inhibiting demyelination, and/or promoting remyelination. For example, an antagonist can interact directly or indirectly with a CXC chemokine and/or CXC chemokine receptor to bring about a reduction in CXC chemokine signaling. Antagonists, as defined herein, without limitation, include oligonucleotide decoys, apatmers, anti-chemokine antibodies and antibody variants, peptides, peptidomimetics, non-peptide small molecules, antisense molecules, and small organic molecules.

The term “agonist” as used herein refers to a molecule having the ability to initiate or enhance a biological function of a target polypeptide. Accordingly, the term “agonist” is defined in the context of the biological role of the target polypeptide. In various embodiments, agonists herein specifically interact with (e.g. bind to) the target, molecules that inhibit a biological activity of the target polypeptide by interacting with other members of the signal transduction pathway of which the target polypeptide is a member are also specifically included within this definition.

For example, one biological activity enhanced by an agonist is associated with increasing proliferation of OPC, inhibiting demyelination, and/or promoting remyelination. Agonists, as defined herein, without limitation, include oligonucleotide decoys, apatmers, anti-chemokine antibodies and antibody variants, peptides, peptidomimetics, non-peptide small molecules, antisense molecules, small organic molecules, and any other biologically active agents disclosed herein.

The term “effective amount” or “therapeutically effective amount” refers to that amount of an agent that is sufficient to effect beneficial or desired results, including without limitation, clinical results such as shrinking the size of demyelinating lesions (in the context of a demyelination disorder, for example), promoting OPC migration, proliferation and growth, delaying the onset of a neuropathy, delaying the development of demyelinating disorder, decreasing symptoms resulting from a neuropathy, increasing the quality of life of those suffering from the disease, decreasing the dose of other medications required to treat the disease, enhancing the effect of another medication such as via targeting and/or internalization, delaying the progression of the disease, decreasing neural scarring, and/or prolonging survival of individuals. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular agent chosen, the dosing regimen to be followed, whether is administered in combination with other compounds, timing of administration, the tissue to be imaged, and the physical delivery system in which it is carried.

The term “antibody” as used herein includes all forms of antibodies such as recombinant antibodies, humanized antibodies, chimeric antibodies, single chain antibodies, humanized antibodies, fusion proteins, monoclonal antibodies etc. The invention is also applicable to antibody functional fragments that are capable of binding to a chemokine (e.g., binding a CXC receptor (CXCR) or a CXCR ligand, such as an interleukin, for example IL-8). An exemplary antibody can increase or decrease the biological activity and/or expression of the target to which it binds.

The terms “modulating”, “modulated” or “modulation” are used interchangeably and mean a direct or indirect change in a given context. For example, modulation of effector T cell proliferation/stimulation means such proliferation can be modulated downward or upward. In another example, modulation can be of the balance of effector or autoreactive T cells or function/activity thereof, versus regulatory T cells of functions/activity thereof.

The term “aptamer” includes DNA, RNA or peptides that are selected based on specific binding properties to a particular molecule. For example, an aptamer(s) can be selected for binding a particular CXCR using methods known in the art. Subsequently, said aptamer(s) can be administered to a subject to modulate or regulate an immune response. Some aptamers having affinity to a specific protein, DNA, amino acid and nucleotides have been described (e.g., K. Y. Wang, et al., Biochemistry 32:1899-1904 (1993); Pitner et al., U.S. Pat. No. 5,691,145; Gold, et al., Ann. Rev. Biochem. 64:763-797 (1995); Szostak et al., U.S. Pat. No. 5,631,146). High affinity and high specificity binding aptamers have been derived from combinatorial libraries (supra, Gold, et al.). Aptamers may have high affinities, with equilibrium dissociation constants ranging from micromolar to sub-nanomolar depending on the selection used. Aptamers may also exhibit high selectivity, for example, showing a thousand fold discrimination between 7-methylG and G (Haller and Sarnow, Proc. Natl. Acad. Sci. USA 94:8521-8526 (1997)) or between D and L-tryptophan (supra, Gold et al.).

The term “decoy” is meant to include a nucleic acid molecule, for example RNA or DNA, or aptamer that is designed to preferentially bind to a predetermined ligand or unknown ligand. Such binding can result in the inhibition or activation of a target molecule. The decoy or aptamer can compete with a naturally occurring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., Cell 63, 601-608 (1990)). This is but a specific example and those in the art will recognize that other embodiments can be readily generated using techniques generally known in the art, see for example Gold et al., Annu. Rev. Biochem., 64, 763-797 (1995); Brody and Gold, J. Biotechnol., 74, 5-13 (2000); Sun, Curr. Opin. Mol. Ther., 2, 100-105 (2000); Kusser, J. Biotechnol., 74, 27-38 (2000); Hermann and Patel, Science, 287, 820-825 (2000); and Jayasena, Clinical Chemistry, 45, 1628-1650 (1999). Similarly, a decoy can be designed to bind to a target antigen to occupy its active site, or a decoy can be designed to bind to a target molecule to prevent interaction with another ligand protein(s), thus short-circuiting a cell signaling pathway that is involved in cell proliferation or differentiation.

### I. Chemokine Signaling

The present invention provides compositions and methods for promoting remyelination by modulating CXCR-mediate signaling. In one aspect, methods of the invention are directed to reducing or eliminating gliosis (e.g., astrogliosis) through blocking or inhibiting CXCR-mediated signaling. Another aspect of the invention is directed to methods for treating a neuropathy by administering one or more agents to a subject, wherein such one or more agents block or inhibit CXCR-mediated signaling. In various embodiments, methods are presented for contacting a cell to promote myelin repairing cells to proliferate, differentiate or migrate.

In other aspects of the present invention, methods are directed to promoting migration of progenitor neural cells (e.g., OPCs) to a lesion site. In some embodiments, methods are provided for administering an agent to a cell or subject to promote myelin repairing cells to migrate to lesion sites. In further embodiments, one or more agents are administered to promote proliferation and/or differentiation of progenitor cells into adult cells (e.g., OPCs into oligodendrocytes). Furthermore, such proliferation and/or differentiation can occur in the CNS or elsewhere (e.g. peripheral nervous system) in an animal. For example, such proliferation and/or differentiation can occur in a hematopoietic context (e.g., stem cells) or in the CNS itself.

In one embodiment, an agent is administered to promote differentiation of oligodendrocyte progenitor cells (OPCs) into mature oligodendrocytes. In some embodiments, a single agent administered can promote migration, proliferation and/or differentiation. In other embodiments, a combination of two or more agents are administered where one agent is effective in migration of a cell, while another agent is effective in proliferation and/or differentiation. In any of such embodiments, the cells are neural cells, or more particularly glial cells.

In other embodiments, such an agent can induce proliferation of cells involved in myelination or cells involved in functional interactions related to myelination. Such cells include but are not limited to OPCs, Schwann cells (SCs), olfactory bulb ensheathing cells, astrocytes, microglia and neural stem cells (NSCs). Thus, for example migration of glial cells may promote migration of OPCs to lesion sites. In one embodiment, one or more agents administered to a subject or contacted to OPCs may promote the migration, proliferation, and/or differentiation of OPCs to/at lesion sites.

Antagonist, agonist and modulators of CXC chemokines and their corresponding receptor function are expressly included within the scope of the invention. Thus, in some aspects of the invention, an agent is administered to effect modulation of chemokine signaling. Chemokines have been shown to regulate oligodendrocyte proliferation and migration. (Robinson et al., J. Neurosci. 18:10457-10463 (1998); Tsai et al., Cell 110:373-383 (2002)). Furthermore, chemokine signaling has been shown to regulate migration, differentiation, activation and proliferation of cells. (Wu et al., J. Neurosci. 20:2609-2617 (2000); Kadi et al., J. Neuroimmunol. 174:133-146 (2006)). Certain chemokine receptors modulate chemokine signaling, and thereby may enhance myelin repair by promoting proliferation of cells involved in myelin repair and/or promoting migration of such cells to the injury or insult site (e.g., lesion site). Some chemokines, such as CXCL1, have been shown to induce OPC proliferation, as well as providing a migratory stop signal during oligodendroglial cell development and positioning them appropriately in the developing CNS (Tsai et al., Cell 110:373-383 (2002)).

In some embodiments, inhibiting CXCR2 signaling promotes increased OPC availability, enhances migration of OPCs or oligodendrocytes into lesion cores and enhances proliferation and/or differentiation. Therefore, chemokine-signaling inhibition can simultaneously preclude damage by preventing immune infiltration and stimulate repair by promoting greater availability of cells involved in myelination/remyelination. For example, an agent can be administered which binds directly/indirectly to a CXCR2, CXCR1 or both CXCR2 and CXCR1 active sites.

The compositions and methods of the present invention may also provide administering an agent to a cell or subject to treat a chemokine-mediated pathology, whereby the agent blocks a chemokine-mediate signaling pathway thus effecting immunomodulation. Immunomodulation includes decreased immune cell infiltration (“immune infiltration”), such as T cell infiltration to the CNS (e.g., autoimmune immune response in MS).

In some aspects of the invention, agents are administered to effect immunomodulation in a combinatorial process, i.e., with myelin repair, remyelination and/or axonal protection, where such immunomodulatory agents include but are not limited to cytokines and cytokine receptors, chemokine and chemokine receptors, antibodies, complement-related biomarkers, adhesion molecules, antigen processing and/or processing markers, cell cycle and apoptosis-related markers. Such factors, receptors and markers are known in the art, and non-limiting examples include, IL-1, IL-2, IL-6, IL-10, IL-12, IL-18, TNF-α, LT-α/β, TGF-β, CCR5, CXCR3, CXCL10, CCR2/CCL2, anti-myelin specific protein/peptide antibodies, anti-cluster of differentiation (CD) antibodies, CSF IgG, anti-MOG antibody, anti-MBP antibody, C3, C4, activated neo-C9, regulators of complement activation, E-selectin, L-selectin, ICAM-1, VCAM-1, LFA-1, VLA-4, heat shock proteins, perforin, OX-40, osteopontin, MRP-8 and MRP-16, neopterin, amyloid A protein, somatostatin, Fas, Fas-L, FLIP, Bcl-2, and TRAIL.

An inhibitor of a CXC-signaling chemokine can lock such a chemokine in an inactive conformation or block the activation site thus preventing the activated receptor-induced intracellular signal transduction cascade and cell response that is necessary to direct immune cells to the CNS. An inhibitor can also be an allosteric inhibitor of a chemokine. Therefore, such an inhibitor can prevent the transduction cascade or cell response that is necessary for immune cell infiltration, for example T cells infiltration, resulting in demyelination. Furthermore, an inhibitor of a CXC-signaling chemokine can be any compound that short-circuits (e.g., prevents or inhibits signaling) the cellular pathways/mechanism required for immune infiltration, thus effecting treatment. In some embodiments, the inhibitor is a peptide, polypeptide, aptamer, siRNA, small organic molecule, pharmaceutical or an antibody, or combinations thereof.

In various embodiments methods are directed to blocking CXCR signaling by administering, in an effective therapeutic amount, an agent that is specific for a CXCR, CXC-ligand (CXCL) or interleukins (IL), where such administration prevents CXC-mediated signaling thus modulating an immune response, including a reduced T cell, B cell or combination T cell/B cell response.

In some embodiments, CXCR signaling by CXCR2 is blocked or inhibited by administering, in an effective therapeutic amount, one or more agent that is specific for CXCR2, or a CXCR2 ligand, such as CXCL1, CXCL2, CXCL3, CXCL5, CXCL6, CXCL7, or CXCL8. In some embodiments, such an agent so administered reduces or eliminates CXCR2 signaling and CXCR1 signaling. In such embodiments, one or more agents selectively block or inhibit CXCR2 and/or CXCR1 as compared to other CXCRs.

In one embodiment, differentially blocking or inhibiting CXCR1 and/or CXCR2 signaling promotes remyelination. In one embodiment, such blocking or inhibiting of CXCR1 and/or CXCR2 signaling reduces or eliminates gliosis.

In some embodiments, a CXCR-inhibiting or blocking agent is polypeptide, peptide, aptamer, antisense molecule, siRNA, ribozyme, peptidomimetic, small organic molecule or chemical compound, or functional variants thereof. In some embodiments, such an agent is specific for a CXCR receptor, CXCR ligand or cognate interleukin. In further embodiments, the agent is specific for CXCR1, CXCR2, CXCR3, CXCL1, CXCL2, CXCL3, CXCL4, CXCL5, CXCL6, CXCL7, CXCL8, or IL-1 to IL-18. In some embodiments, an agent is an antagonist for CXCR1, CXCR2, CXCR3, CXCL1, CXCL5, CXCL8 or IL-8, whereby administration of such an agent inhibits CXC-mediated signaling. In one embodiment, such an agent selectively blocks/inhibits CXCR1 relative to other CXCRs. In one embodiment, such an agent selectively blocks/inhibits CXCR2 relative to other CXCRs. In another embodiment, such an agent selectively blocks or inhibits CXCR1 and/or CXCR2 relative to other CXCRs. In yet further embodiments, two or more agents are administered which can selectively block or inhibit CXCR1 and/or CXCR2 as compared to other CXCRs.

The methods disclosed herein can be directed to any neuropathological condition, for example, where degeneration of neural cells occurs or demyelination. Neuronal demyelination is manifested in a large number of hereditary and acquired disorders of the CNS and PNS. Neuropathologies include, but are not limited to, Multiple Sclerosis (MS), Progressive Multifocal Leukoencephalopathy (PML), Encephalomyelitis, Central Pontine Myelolysis (CPM), Anti-MAG Disease, Leukodystrophies: Adrenoleukodystrophy (ALD), Alexander's Disease, Canavan Disease, Krabbe Disease, Metachromatic Leukodystrophy (MLD), Pelizaeus-Merzbacher Disease, Refsum Disease, Cockayne Syndrome, Van der Knapp Syndrome, and Zellweger Syndrome, Guillain-Barre Syndrome (GBS), chronic inflammatory demyelinating polyneuropathy (CIDP), multifocal motor neuropathy (MMN), spinal chord injury (e.g., trauma or severing of), Alzheimer's Disease, Huntington's Disease, Amyotrophic Lateral Sclerosis, Parkinson's Disease, gliosis, astrogliosis and optic neuritis, which have been linked to the degeneration of neural cells in particular locations of the CNS, leading to the inability of neural cells or the brain region to carry out their intended function. In addition, the methods disclosed herein are equally applicable to neuropathy caused by or associated with pathogens including but not limited to pathogens causing measles, rabies, scrapie-like agent, Carp agent, paramyxovirus, coronavirus, Epstein-Barr virus, herpes zoster, herpes simplex virus, human herpesvirus 6, rubella, mumps, canine distemper, Marek's Semliki forest virus, animal and human retroviruses, and human T cell lymphoma virus type I.

### II. Bioactive Agents

The compositions and methods disclosed herein can be directed to any neuropathological condition, for example, where degeneration of neural cells occurs, gliosis (e.g., astrogliosis) or demyelination. Bioactive agents can be directed towards such neuropathological conditions. Bioactive agents can be agonists or antagonists of chemokine receptors or ligands. In some embodiments of the invention, it is envisioned that compounds having the same three dimensional structure at the binding site may be used as antagonists. Three dimensional analysis of chemical structure is used to determine the structure of active sites, including binding sites for chemokines. For example, chemical leads with high throughput screening have been used to generate and chemically optimize a selective antagonist of the CXCR2 (Ganju et al., J. Biol Chem, 273:10095-10098 (1998)). In addition, nuclear magnetic resonance spectroscopy (NMR) may be utilized to detail the three dimensional structure of ligands for CXCRs, including both ELR and non-ELR CXC chemokines. With NMR information, multiple substitutions can be generated within the receptor binding sites of multiple chemokines, such that they could substantially alter the ligands' receptor specificities. (e.g., Wells et al. J. Leuk. Biol. 1996; 59:53-60). Therefore, agents that block or inhibit chemokine-mediated signaling can be designed for use in methods of the invention, including treating a neuropathy. Furthermore, as disclosed herein, such agents can be used in methods of reducing or eliminating gliosis, as well as promoting remyelination.

In another aspect of the invention, a bioactive agent(s) can be administered in a therapeutically effective amount to modulate expression of CXC chemokines thereby affecting CXC-mediated signaling. Such modulation can in turn affect glial cell migration, proliferation and/or differentiation. In a further embodiment, such modulation can affect immune cell infiltration into the CNS.

In various embodiments, such agents include, without being limited to, peptides, polypeptides, antisense molecules, aptamers, siRNAs, external guide sequence (EGS) small organic molecules, antibodies or peptidomimetics. Such bioactive agents can directly or indirectly modulate expression levels of CXC chemokines, thus reducing or enhancing CXC-mediated signaling. In various embodiments, CXC-chemokines whose expression is modulated include CXCL1, CXCL5, CXCL8, CXCR1, CXCR2 or CXCR5. In some embodiments, expression of CXCL1 and/or CXCR2 is modulated. In some embodiments, glial cells are cultured and transfected with expression constructs in vitro and subsequently administered to a subject, wherein the expression constructs encode CXCR1 and/or CXCR2. Therefore, in some embodiments, modulated expression is effected through ex vivo methods.

In other aspects of a target cell can be engineered to express one more additional agents that promote myelin repair. For example, in some embodiments, an agent, such as a myelin repair promoting nerve growth factor, for example, is encoded by a nucleic acid sequence that is transformed into a target cell. Therefore, the desired growth factor is expressed from the nucleic acid sequence which can be integrated into the cell genome, or present on a plasmid or viral vector, which are known in the art.

Therefore, in some embodiments, nucleic acids encoding an agent that modulates CXCR-mediated signaling can be co-administered with nucleic acids encoding an agent that promotes remyelination in a combinatorial fashion. For example, two or more co-administered agents expressed from the nucleic acid may promote migration, proliferation, and/or differentiation of glial cells, as well as inhibit or reduce gliosis. The agent expressed from the nucleic acid may block or inhibit CXC signaling, for example, by inhibiting CXCR2 activity or CXCR2 ligands. In other embodiments, such agents block or inhibit CXCR1- and CXCR2-mediated signaling. In yet other embodiments, such agents block or inhibit CXCR1-mediated signaling. Thus, agents inhibiting or blocking CXCR-mediated (or CXCL-mediate) signaling can be combined with agents promoting myelin repair, such as through enhancing or promoting oligodendrocyte survival.

Such agents to be co-administered with agents affected CXCR-mediates signaling include several biological molecules, which have been shown to influence the processes of oligodendrocyte survival, proliferation, migration and differentiation, such as Platelet Derived Growth Factor (PDGF) (Jean et al., Neuroreport 13:627-631 (2002)), Thyroid Hormone (TH) (Calza et al., Proc. Natl. Acad. Sci. USA 99:3258-3263 (2002)), Granulocyte Colony Stimulating Factor (GCSF) (Zavala et al., J. Immunol. 168:2011-2019. (2002)), Ciliary Neurotrophic Factor (CNTF) (Linker et al., Nat. Med. 8:620-624 (2002)), Fibroblast Growth Factor-2 (FGF-2) (Armstrong et al., J. Neurosci. 22:8574-8585 (2002).), Leukemia Inhibitory Factor (LIF) (Butzkueven et al., Nat. Med. 8:613-619 (2002).), Insulin Like Growth Factor-1 (IGF-1) (Beck et al., Neuron 14:717-730 (1995)), Glial Growth Factor-2/Neuregulin (GGF-2/NRG) (Kerber et al., J. Mol. Neurosci. 21:149-165 (2003)) and CXCL1/Growth Regulated Oncogene Alpha (Gro-α) (Omari et al., Glia 53:24-31 (2006); Omari et al., Brain 128:1003-1015 (2005); Tsai et al., Cell 110:373-383 (2002)).

In some further embodiments, the expression of a nucleic acid sequence encoding such an agent is inducible thus temporally controlled. Such inducible or temporally controlled transcription regulatory elements are known in the art and as further disclosed herein.

In some embodiments, glial cells can be transfected with an expression vector (or “expression construct”) that encodes a bioactive agent so as to provide altered expression of a CXC chemokine. In one embodiment, expression constructs are transfected into oligodendrocytes, whereby such expression vectors are administered to cells in vitro or in vivo, and where a transfected oligodendrocyte produces altered expression levels of CXCR2 or CXCL1 as compared to non-transfected oligodendrocyte. In other embodiments, such transfected cells include SCs, NSCs, OPCs, astrocytes, microglial cells or a combination of such cells, which are also transfected in culture or in vivo. In some embodiments, the expression constructs comprise cell-specific or inducible promoters, which are specific for glial cells, and are described herein above, as well as known to one of ordinary skill in the art.

Typically, gene expression is placed under the control of certain regulatory elements, including constitutive or inducible promoters, tissue-specific regulatory elements, and enhancers. Such a gene is said to be “operably linked to” the regulatory elements. For example, constitutive, inducible or cell/tissue specific promoters can be incorporated into an expression vector to regulate expression of a gene that is expressed in a host cell.

In some embodiments, an inhibitor of CXCR signaling is a polypeptide, which can be expressed from nucleic acid sequences encoding such an inhibitor, whereby a nucleic acid encoding the inhibitor can be operably linked to transcription regulatory sequences that are specific to neural cells. Exemplary transcriptional regulatory sequences/elements include transcriptional regulatory sequences/elements selected from the genes encoding the following proteins: the PDGFα receptor, proteolipid protein (PLP), the glial fibrillary acidic gene (GFAP), myelin basic protein (MBP), neuron specific enolase (NSE), oligodendrocyte specific protein (OSP), myelin oligodendrocyte glycoprotein (MOG) and microtubule-associated protein 1B (MAP1B), Thy 1.2, CC1, ceramide galactosyltransferase (CGT), myelin associated glycoprotein (MAG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), tyrosine hydroxylase, BSF1, dopamine 3-hydroxylase, Serotonin 2 receptor, choline acetyltransferase, galactocerebroside (GalC), and sulfatide. Furthermore, examples of neural cell-specific promoters are known in the art, such as disclosed in U.S. Patent Application Publication No. 2003/0110524; See also, the website <chinook.uoregon.edu/promoters.html>. Additionally, cell/tissue specific promoters are also known in the art.

In some embodiment, the transcriptional regulatory elements are inducible. For example, non-limiting examples of inducible promoters include metallothionine promoters and mouse mammary tumor virus promoters. Other examples of promoters and enhancers effective for use in the recombinant vectors of the present invention include, but are not limited to, CMV (cytomegalovirus), SV40 (simian virus 40), HSV (herpes simplex virus), EBV (Epstein-Barr virus), retrovirus, adenoviral promoters and enhancers, and smooth-muscle-specific promoters and enhancers; strong constitutive promoters that may be suitable for use as the heterologous promoter include the adenovirus major later promoter, the cytomegalovirus immediate early promoter, the β-actin promoter, or the β-globin promoter. Promoters activated by RNA polymerase III could also be used.

In some embodiments, inducible promoters that have been used to control gene expression include the tetracycline operons, RU 486, heavy metal ion inducible promoters such as the metallothionein promoter; steroid hormone inducible promoters, such as the MMTV promoter, or the growth hormone promoter; promoters which would be inducible by the helper virus such as adenovirus early gene promoter inducible by adenovirus E1A protein, or the adenovirus major late promoter; herpesvirus promoter inducible by herpesvirus proteins such as VP16 or 1CP4; vaccinia or poxvirus inducible promoters or promoters inducible by a poxvirus RNA polymerase; bacterial promoter such as that from T7 phage which would be inducible by a poxvirus RNA polymerase; or a bacterial promoter such as that from T7 RNA polymerase, or ecdysone. In one embodiment, a promoter element is a hypoxic response elements (HRE) recognized by a hypoxia-inducible factor-1 (HIF-1) which is one of the key mammalian transcription factors that exhibit dramatic increases in both protein stability and intrinsic transcriptional potency during low-oxygen stress. HRE has been reported in the 5′ or 3′ flanking regions of VEGF and Epo and several other genes. The core consensus sequence is (A/G)CGT(G/C)C. HREs isolated from Epo and VEGF genes have been used to regulate several genes, such as suicide gene and apoptosis gene expression in hypoxic tumors to enhance tumor killing.

Furthermore, where expression of the transgene in particular subcellular location is desired, the transgene can be operably linked to the corresponding subcellular localization sequences by recombinant DNA techniques widely practiced in the art. Exemplary subcellular localization sequences include but are not limited to (a) a signal sequence that directs secretion of the gene product outside of the cell; (b) a membrane anchorage domain that allows attachment of the protein to the plasma membrane or other membraneous compartment of the cell; (c) a nuclear localization sequence that mediates the translocation of the encoded protein to the nucleus; (d) an endoplasmic reticulum retention sequence (e.g. KDEL sequence) that confines the encoded protein primarily to the ER; (e) proteins can be designed to be farnesylated so as to associate the protein with cell membranes; or (f) any other sequences that play a role in differential subcellular distribution of a encoded protein product.

Vectors utilized in in vivo or in vitro methods can include derivatives of SV-40, adenovirus, retrovirus-derived DNA sequences and shuttle vectors derived from combinations of functional mammalian vectors and functional plasmids and phage DNA. Eukaryotic expression vectors are well known, e.g. such as those described by Southern and Berg, J. Mol. Appl. Genet. 1:327-341 (1982); Subramini et al., Mol. Cell. Biol. 1:854-864 (1981), Kaufmann and Sharp, J. Mol. Biol. 159:601-621 (1982); Scahill et al., Proc. Natl. Acad. Sci. USA 80:4654-4659 (1983) and Urlaub and Chasin Proc. Natl. Acad. Sci. USA 77:4216-4220 (1980), which are hereby incorporated by reference. The vector used in the methods of the present invention may be a viral vector, preferably a retroviral vector. Replication deficient adenoviruses are preferred. For example, a “single gene vector” in which the structural genes of a retrovirus are replaced by a single gene of interest, under the control of the viral regulatory sequences contained in the long terminal repeat, may be used, e.g. Moloney murine leukemia virus (MoMulV), the Harvey murine sarcoma virus (HaMuSV), murine mammary tumor virus (MuMTV) and the murine myeloproliferative sarcoma virus (MuMPSV), and avian retroviruses such as reticuloendotheliosis virus (Rev) and Rous Sarcoma Virus (RSV), as described by Eglitis and Andersen, BioTechniques 6:608-614 (1988), which is hereby incorporated by reference.

Recombinant retroviral vectors into which multiple genes may be introduced may also be used according to the methods of the present invention. Vectors with internal promoters containing a cDNA under the regulation of an independent promoter, e.g. SAX vector derived from N2 vector with a selectable marker (neo^{R}) into which the cDNA for human adenosine deaminase (hADA) has been inserted with its own regulatory sequences, the early promoter from SV40 virus (SV40), may be designed and used in accordance with the methods of the present invention by methods known in the art.

In mammalian host cells, a number of viral-based expression systems can be utilized. In cases where an adenovirus is used as an expression vector, the nucleotide sequence of interest (e.g., encoding a therapeutic capable agent) can be ligated to an adenovirus transcription or translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the AQP1 gene product in infected hosts. (See e.g., Logan & Shenk, Proc. Natl. Acad. Sci. USA 8 1:3655-3659 (1984)).

Specific initiation signals can also be required for efficient translation of inserted therapeutic nucleotide sequences. These signals include the ATG initiation codon and adjacent sequences. In cases where an entire therapeutic gene or cDNA, including its own initiation codon and adjacent sequences, is inserted into the appropriate expression vector, no additional translational control signals may be needed. However, in cases where only a portion of the therapeutic coding sequence is inserted, exogenous translational control signals, including, perhaps, the ATG initiation codon, may be provided. Furthermore, the initiation codon may be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (See e.g., Bittner et al., Methods in Enzymol, 153:516-544 (1987)).

In some embodiments neural cells, such as glial cells are genetically modified by utilization of the foregoing vectors, so as to produce at an altered level bioactive agents that are directed to blocking or inhibiting CXCR signaling. In one embodiment, such agents block or inhibit CXCR1 and/or CXCR2 signaling. Genetically modifying or transfecting cells either in vitro or in vivo can be conducted utilizing methods known in the art, as described in references noted herein above, and such as disclosed in U.S. Pat. Nos. 6,998,118; 6,670,147 or 6,465,246.

In some embodiments, a bioactive agent is administered and can result in altered expression levels of one or more proteins involved in CXC chemokine-mediated signaling. Alternatively, neural cells as described herein can be modified, for example, by contacting neural cells with a bioactive agent, to provide altered expression of one or more proteins involved in CXC chemokine-mediated signaling. In one embodiment, such signaling is CXCR1 and/or CXCR2 mediated signaling. Furthermore, methods of measuring expression levels of polypeptides are well known to one of ordinary skill in the art.

Examples of neural cells used in one or more methods of the invention include glial cells. Glia are subdivided into macroglia, which consist of astrocytes, oligodendrocytes, and microglia. Within the microenvironment of the CNS, astrocytes provide support and nourishment, oligodendrocytes provide insulation, and microglia provide immune defense. Astrocytes, commonly identified by the expression of the intermediate filament protein glial fibrillary acidic protein (GFAP), possess a variety of ion channels, transporters, and neurotransmitter receptors that help maintain brain homeostasis and may alter neuronal excitability. In addition, astrocytes interact with endothelial cells, and these interactions are thought to be critical for the development and maintenance of the blood brain barrier (BBB). Astrocytes are known to react to CNS injury by proliferating, changing their morphology, expanding processes, and enhancing their expression of GFAP. This activation, termed astrocytosis or astrogliosis, may lead to deposition of extracellular matrix molecules (ECM) into a dense fibrous scar. Such a response to injury is considered detrimental for repair. Furthermore, following injury, astrocytes can activate glutamate receptors leading to excitotoxicity and death of surrounding cells.

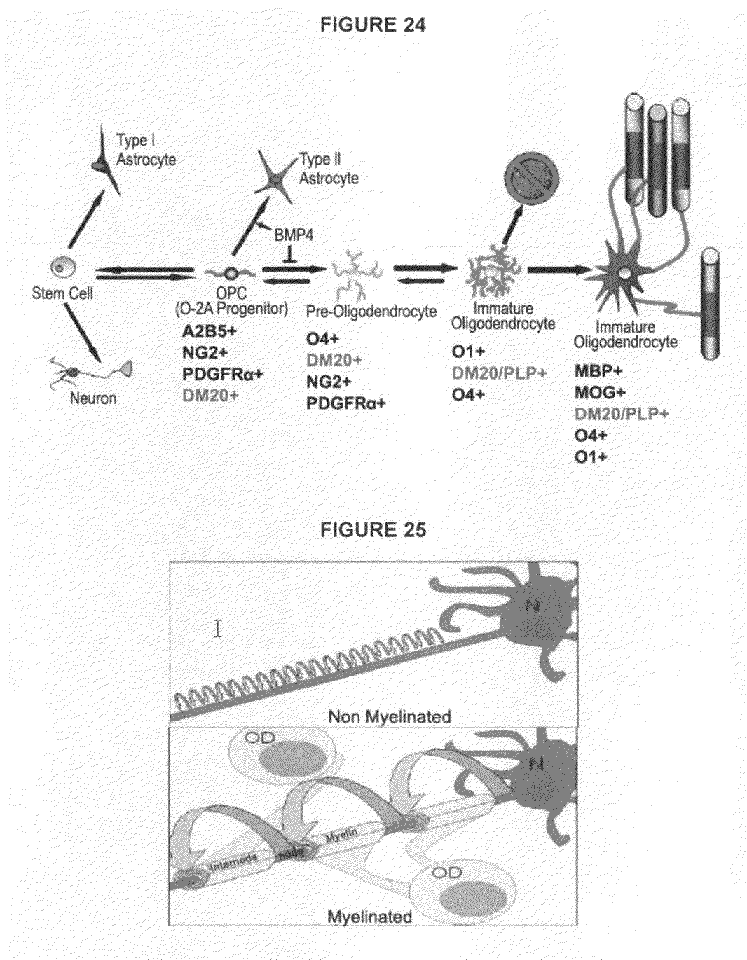

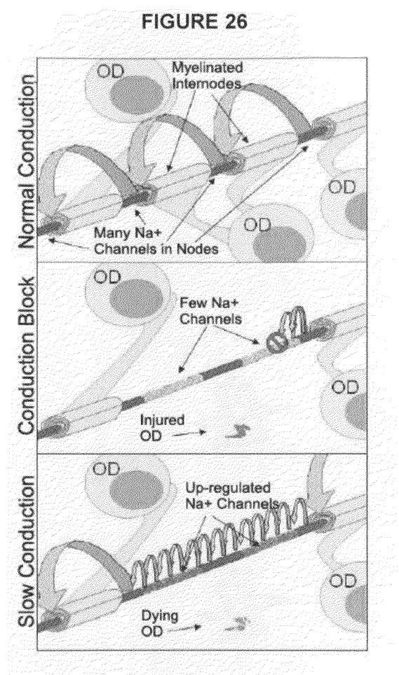

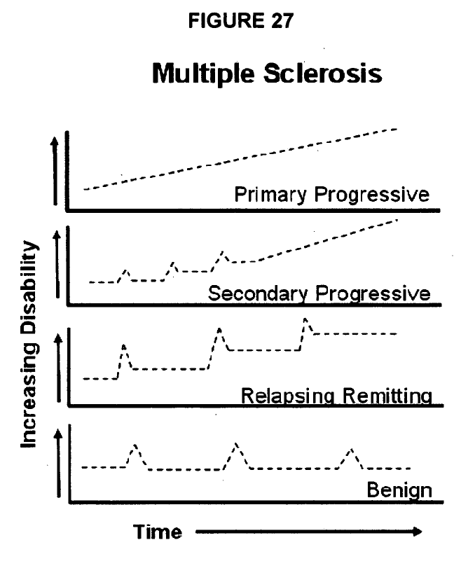

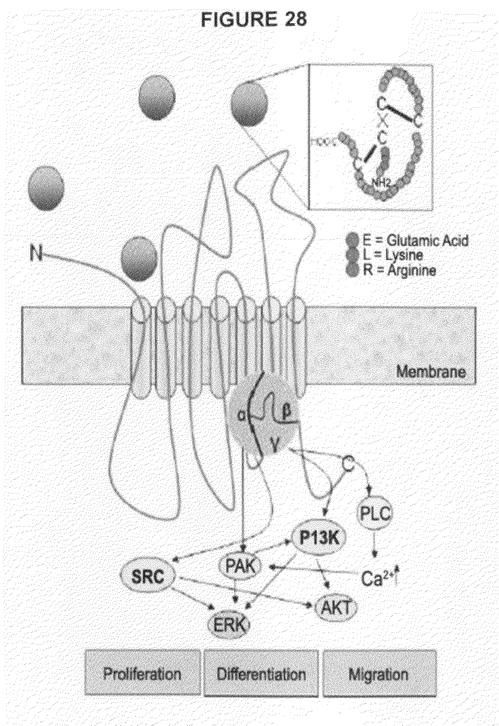

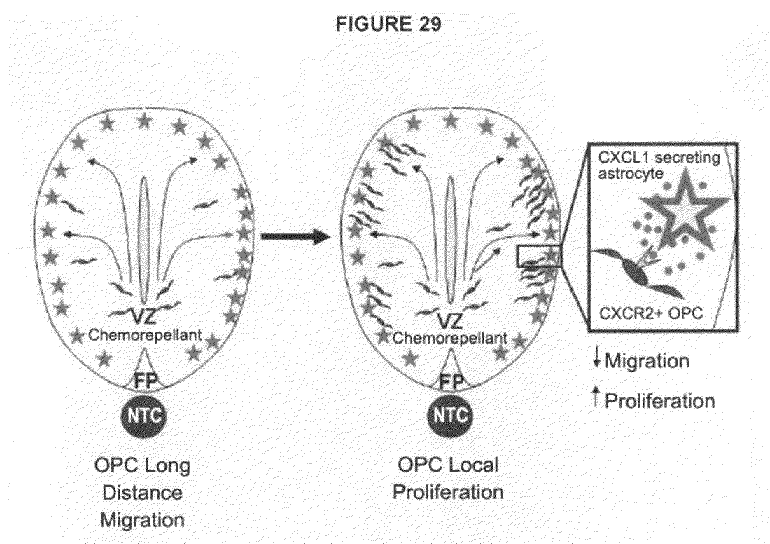

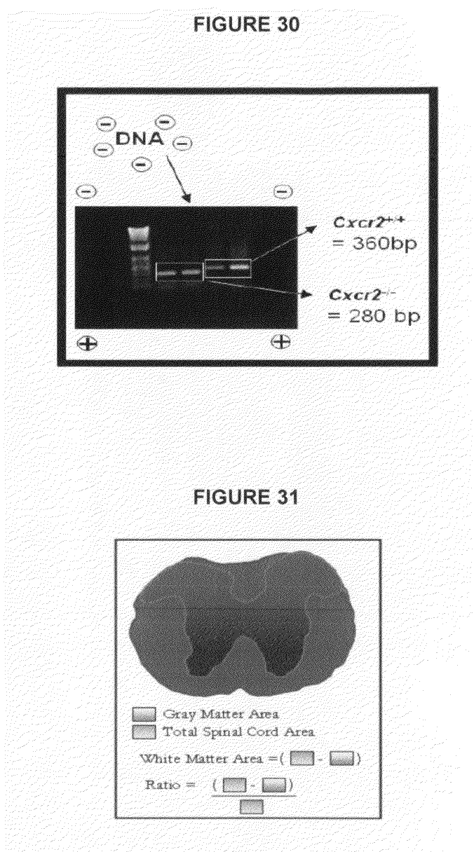

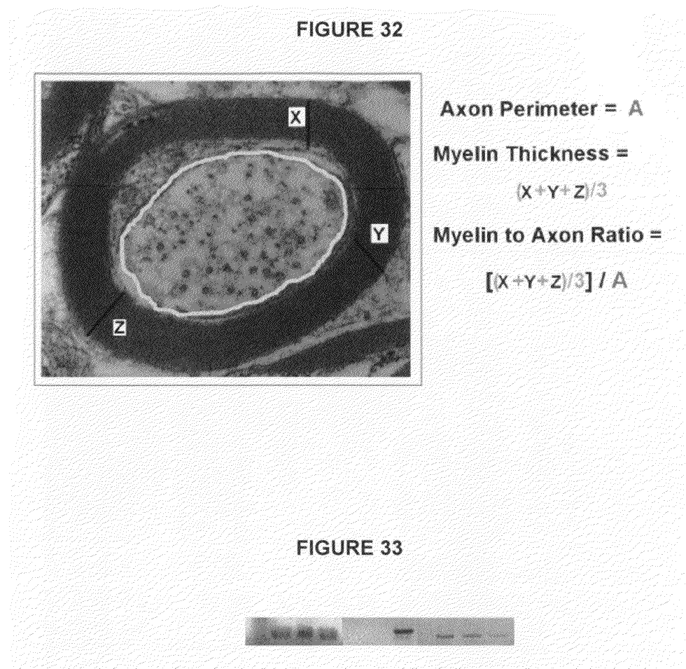

Neural cells of the present invention also includes oligodendrocytes, which are the macroglial cells typically responsible for the production and maintenance of CNS myelin, the fatty insulation that enwraps axons to enhance the speed and reliability with which information is transmitted. Oligodendrocytes typically first develop in the CNS from the ventral ventricular and subventricular zones of the spinal cord and brain. Oligodendrocytes in the spinal cord typically arise from the ventricular zone during embryonic development and subsequently migrate to white matter where they proliferate and differentiate (Miller, Prog. Neurobiol. 67:451-467 (2002)) (FIG. 23). During their maturation and differentiation, oligodendrocytes typically go through a sequence of developmental stages characterized by distinct alterations in cell morphology and the expression of specific molecular markers (FIG. 24). The specificity of these markers for individual cell populations allows identification of cells-at different stages and opportunities for their isolation.

In some embodiments, glia cells are microglia, which as the name suggests, are the smallest of the three CNS glial cells and share characteristics with bone marrow derived monocytes and macrophages to which they are related. They are derived from myeloid progenitor cells of lymphoid tissues and are thought to arrive to the CNS during its developmental vascularization. Resting microglia have elongated bipolar cell bodies with perpendicular spine-like processes. Microglia are highly motile cells and, when activated, are thought to act like immune cells in the CNS, with phagocytosis, presentation of antigens, and secretion of inflammatory cytokines. Astrocytes and microglia may act as antigen presenting cells and that this behavior may amplify immune responses and lead to uncontrolled myelin destruction.

In certain embodiments, one or more agents utilized in methods of the invention (e.g., agonists, antagonists, or modulators) are antibodies and immunoglobulin variants that bind to CXC chemokine and its corresponding receptor. These agents can be provided in linear or cyclized form, and optionally comprise at least one amino acid residue that is not commonly found in nature or at least one amide isostere. These compounds may be modified by glycosylation, phosphorylation, sulfation, lipidation or other processes.

In various embodiments, certain chemokines targeted by such antibodies are ELR chemokines. The ‘ELR’ chemokines chemoattract and activate inflammatory cells via their CXCR1 and CXCR2 receptors (Baggiolini, Nature 392:565-568 (1998); Ahuja and Murphy, J. Biol. Chem. 271:20545-20550 (1996)). The CXCR1 is typically specific for CXCL8 and CXCL6/granulocyte chemotactic protein-2 (GCP-2), while the CXCR2 typically binds CXCL8 with high affinity, but also macrophage inflammatory protein-2 (MIP-2), CXCL1, CXCL5/ENA-78, and CXCL6 with somewhat lower affinities (see, for example, Baggiolini and Moser, Rev. Immunol., 15:675-605 (1997)). CXCL8 signaling in cell lines transfected with the human CXCR1 or CXCR2 generally induces equipotent chemotactic responses (Wuyts et al., Eur. J. Biochem. 255:67-73 (1998); Richardson et al., J. Biol. Chem. 273:23830-23836 (1998)).

In various embodiments, anti-CXCR or anti-CXCL antibodies are administered to a subject to modulate CXC-mediated signaling resulting in enhanced glial cell proliferation and/or differentiation, and/or reduced immune cell infiltration. In one embodiment, such antibodies reduce CXC-mediated signaling, e.g., via inhibiting the activity or expression level of CXCR or CXCL. In some embodiments, in methods of inhibiting CXC-signaling, an antibody is administered to a cell/subject which antibody can be specific for CXCR1, CXCR2, CXCR3, ELR⁺CXC chemokine, CXCL1, CXCL2, CXCL3, CXCL4, CXCL5, CXCL6, CXCL7 or CXCL8. In various embodiments, an antibody is specific for CXCR2, CXCR3, CXCL1, CXCL8, CXCL6 or IL-8. In one embodiment, the antibody is AMX-IL-8 (Abgenix). (Mahler et al., Chest 126:926-934 (2004)). In another embodiment, the antibody is specific for CXCL1, CXCR1, or CXCR2. In some embodiments, one or more agents block or inhibit CXCR1 and/or CXCR2 signaling. In another embodiment, such one or more agents block or inhibit CXCLs, including but not limited to CXCL1, CXCL5 or CXCL8. In one embodiment, one or more agents are administered to block CXCR1, CXCR2, CXCL1, CXCL5, CXCL8, or a combination thereof.

Producing antibodies specific for chemokine antigens described herein is known to one of skill in the art, such as disclosed in U.S. Pat. Nos. 6,491,916; 6,982,321; 5,585,097; 5,846,534; 6,966,424 and U.S. Patent Application Publication Nos. 20050054832; 20040006216; 20030108548; 2006002921 and 20040166099, each relevant portion of which is incorporated herein by reference. In merely one example, monoclonal antibodies can be obtained by injecting mice with a composition comprising the antigen, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen that was injected, culturing the clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, for example, Coligan at pages 2.7.1 2.7.12 and pages 2.9.1 2.9.3. Also, see Baines et al., “Purification of Immunoglobulin G (IgG),” in METHODS IN MOLECULAR BIOLOGY, VOL. 10, pages 79 104 (The Humana Press, Inc. 1992).

Suitable amounts of well-characterized antigen for production of antibodies can be obtained using standard techniques. As an example, chemokine antigen can be immunoprecipitated from cells using the deposited antibodies described by Tedder et al., U.S. Pat. No. 5,484,892 (1996). Alternatively, chemokine antigen proteins can be obtained from transfected cultured cells that overproduce the antigen of interest. Expression vectors that comprise DNA molecules encoding each of these proteins can be constructed using published nucleotide sequences. See, for example, Wilson et al., J. Exp. Med. 173:137-146 (1991); Wilson et al., J. Immunol. 150:5013-5024 (1993). As an illustration, DNA molecules encoding CD3 can be obtained by synthesizing DNA molecules using mutually priming long oligonucleotides. See, for example, Ausubel et al., (eds.), CURRENT PROTOCOLS IN MOLECULAR BIOLOGY, pages 8.2.8 to 8.2.13 (1990). Also, see Wosnick et al., Gene 60:115-127 (1987); and Ausubel et al. (eds.), SHORT PROTOCOLS IN MOLECULAR BIOLOGY, 3rd Edition, pages 8-8 to 8-9 (John Wiley & Sons, Inc. 1995). Established techniques using the polymerase chain reaction provide the ability to synthesize genes as large as 1.8 kilobases in length. (Adang et al., Plant Molec. Biol. 21:1131-1145 (1993); Bambot et al., PCR Methods and Applications 2:266-271 (1993); Dillon et al., “Use of the Polymerase Chain Reaction for the Rapid Construction of Synthetic Genes,” in METHODS IN MOLECULAR BIOLOGY, Vol. 15: PCR PROTOCOLS: CURRENT METHODS AND APPLICATIONS, White (ed.), pages 263 268, (Humana Press, Inc. 1993)). In a variation, monoclonal antibody can be obtained by fusing myeloma cells with spleen cells from mice immunized with a murine pre-B cell line stably transfected with cDNA which encodes the antigen of interest. (See Tedder et al., U.S. Pat. No. 5,484,892.)

In one embodiment, comparatively low doses of an entire, naked antibody or combination of entire, naked antibodies are used. In some embodiments, antibody fragments are utilized, thus less than the complete antibody. In other embodiments, conjugates of antibodies with drugs, toxins or therapeutic radioisotopes are useful. Bispecific antibody fusion proteins which bind to the chemokine antigens can be used according to the present invention, including hybrid antibodies which bind to more than one antigen. Preferably the bispecific and hybrid antibodies additionally target a T-cell, plasma cell or macrophage antigen. Therefore, antibody encompasses naked antibodies and conjugated antibodies and antibody fragments, which may be monospecific or multispecific.

Marker proteins and functional fragments thereof may be, utilized as antigen-specific immunomodulation components in one or more method of the invention to induce antigen-specific tolerance, which in combination with an agent delivered to induce myelin repair can result in a synergistic therapeutic result. Non-limiting exemplary marker proteins of a myelinating cell (including oligodendrocyte and Schwann cell) may be selected from the group consisting of CC1, myelin basic protein (MBP), ceramide galactosyltransferase (CGT), myelin associated glycoprotein (MAG), myelin oligodendrocyte glycoprotein (MOG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), galactocerebroside (GalC), sulfatide and proteolipid protein (PLP). MPZ, PMP22 and P2 are some markers for Schwann cells.

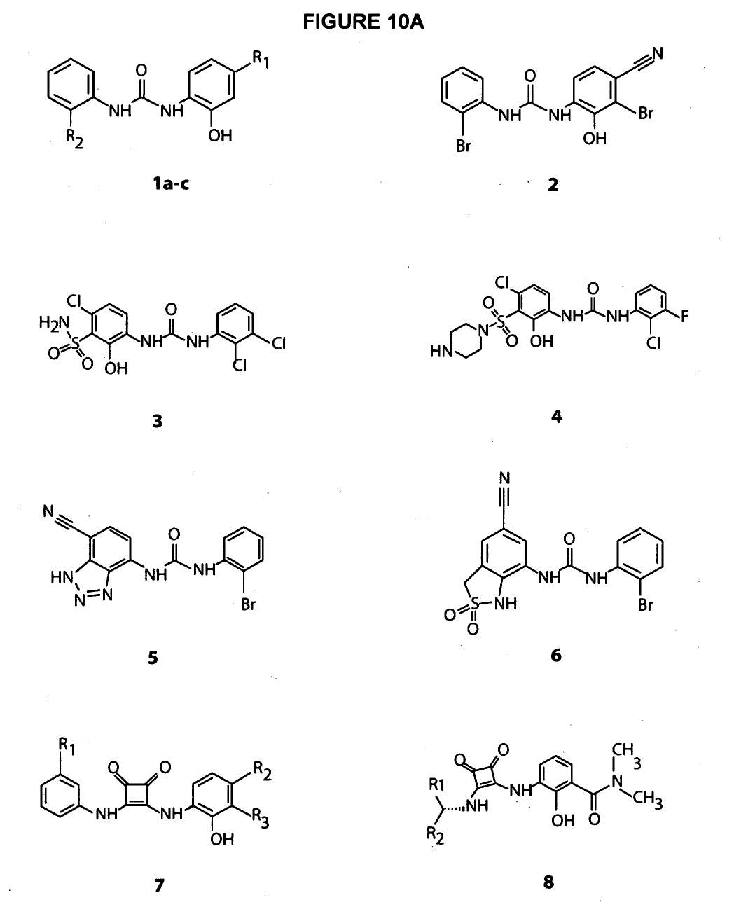

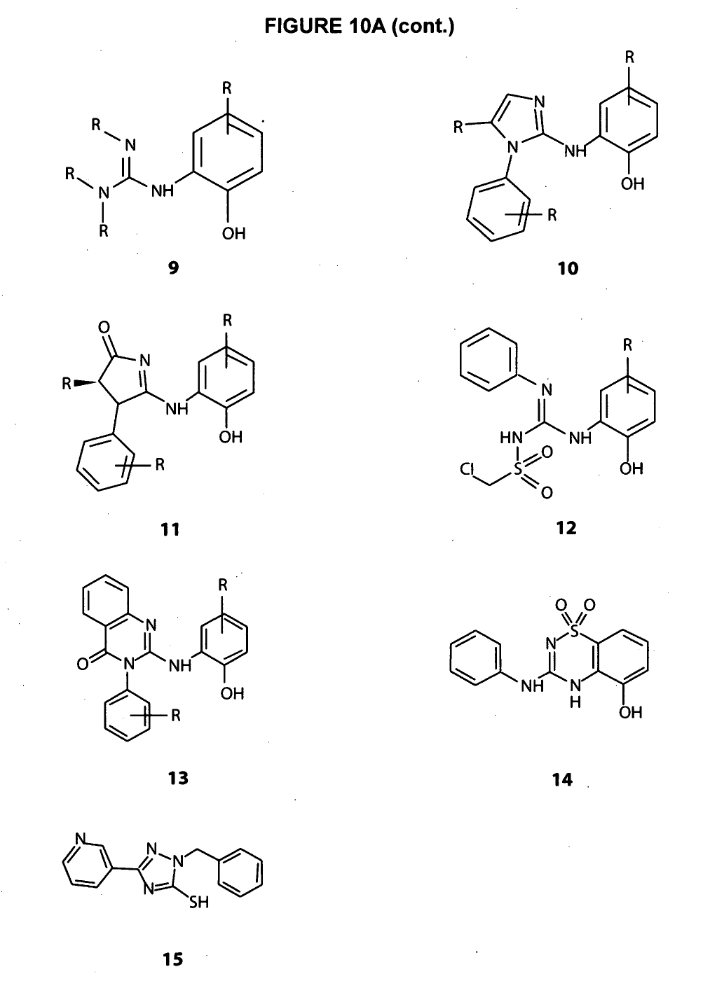

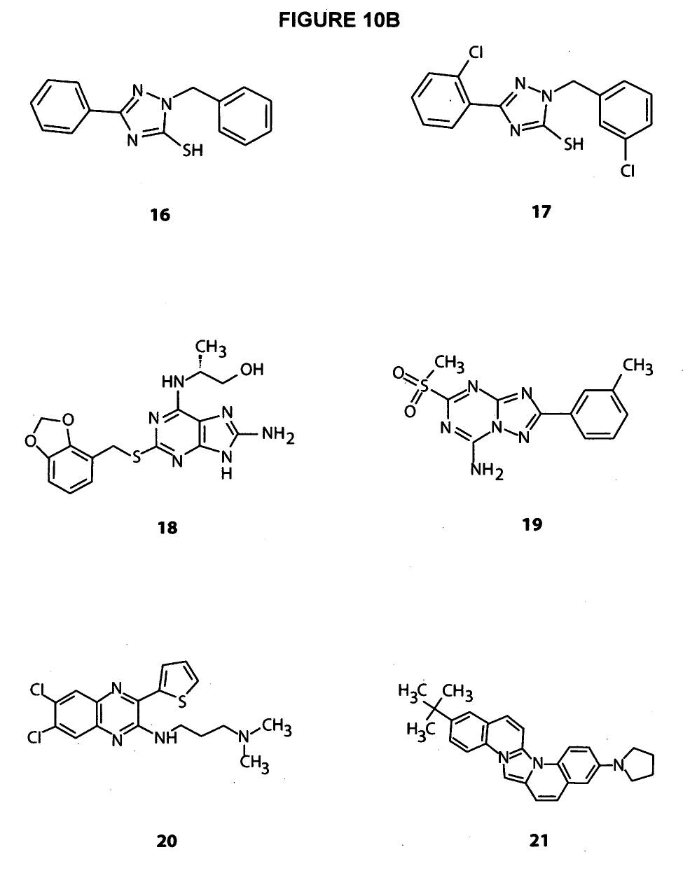

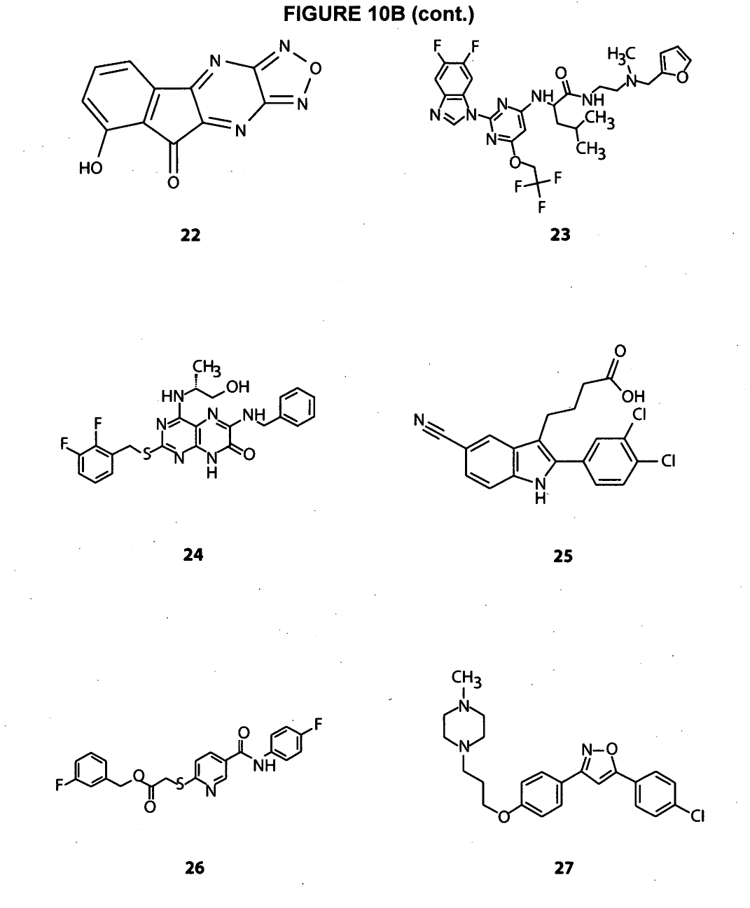

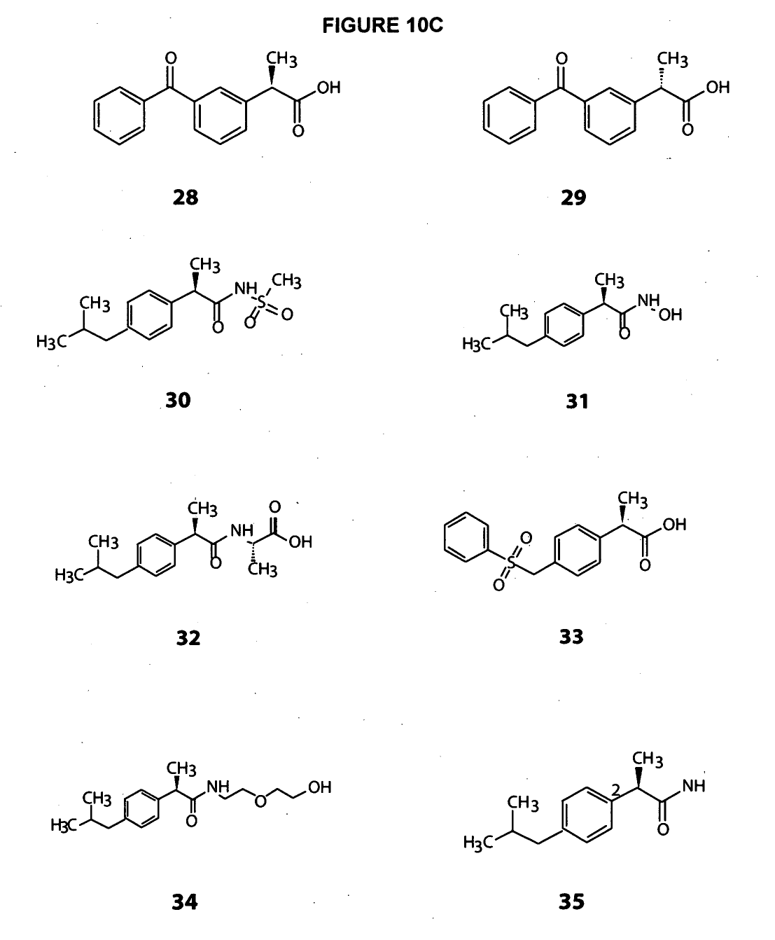

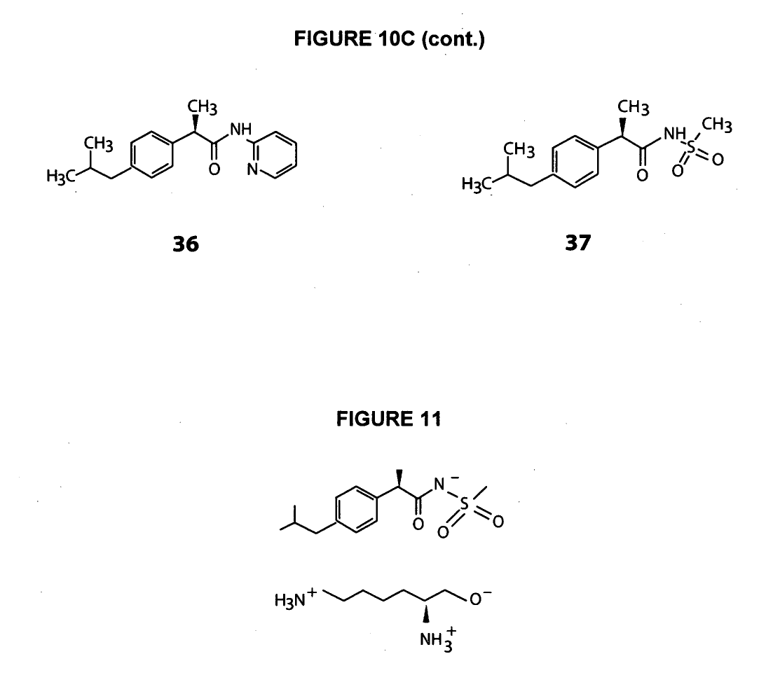

In some embodiments, a chemokine-signaling inhibitor is a small molecule, including but not limited to repertaxin (FIG. 11), CXCR1/CXCR2 antagonists (FIGS. 10A-10B) (Bizzarri et al., Pharmacology. & Therapeutics, 112:139-149 (2006)), CXCR1/CXCR2 allosteric inhibitors (FIG. 10C) (Bizzarri et al., Pharmacology. & Therapeutics, 112:139-149 (2006)) and IL-8 antagonists such as dianilino squarates disclosed in U.S. Pat. No. 7,008,962. Other inhibitors may include 3,4,5-trisubstituted aryl nitrones (U.S. Pub. No. 20050215646) and CXCR1 and CXCR2 antagonists disclosed in U.S. Pub. No. 20060014794. In some embodiments, the bioactive agent may be IL-8 mimetics, such as disclosed in U.S. Pub. No. 20060233748 (peptides) or sRAGE as disclosed in U.S. Pub. No. 20070167360.

### III. Cell Transplanation

In another aspect of the invention, cells involved in myelin repair or remyelination of denuded axons are administered to a subject, wherein said cells are modified to have decreased CXC signaling, for example, decreased CXCR2 signaling. Such cells can be cultured and transfected with an appropriate vector to express a polypeptide that leads to enhanced cell proliferation, differentiation, or migration to an injury or insult site (e.g., demyelinated site). In some embodiments, cells involved in remyelination or myelin repair are modified to have decreased CXC ligand expression, such as decreased CXCL1, CXCL2, CXCL3, CXCL4, CXCL5, CXCL6, CXCL7, or CXCL8 expression. In other embodiments, the cells have decreased CXC receptor expression. In some embodiments, the cell is modified to block or inhibit CXCR2 or its ligands, e.g., CXCL1. In one embodiment, the cell is modified to block or inhibit CXCR1 and/or CXCR2.

In various embodiments, the cells (“cell types”) are those disclosed herein. For example, such cells are oligodendrocyte progenitor cells (OPCs), Schwann cells (SCs), olfactory bulb ensheathing cells, astrocytes, microglia and neural stem cells (NSCs). In some embodiments, one or more cell types are modified to inhibit chemokine expression and chemokine receptor expression and administered to a subject to treat a neuropathy. In one embodiment, two cell types are administered including OPCs and astrocytes. In some embodiments, an agent is administered to promote differentiation of oligodendrocyte progenitor cells (OPCs) into mature oligodendrocytes. Alternatively, such an agent can induce proliferation of cells involved in myelination or cells involved in functional interactions related to myelination, such neural cells include but are not limited to OPCs, Schwann cells (SCs), olfactory bulb ensheathing cells, astrocytes, microglia and neural stem cells (NSCs). An agent may also induce migration of glial cells such as OPCs. Such neural cells may be administered prior to, concurrent with, or subsequent to administration of a bioactive agent. In other embodiments, one or more types of neural cells can be administered with one or more types of bioactive agents. For clarity, the term “type” means for example, different types of cells (e.g., oligodendrocyte and astrocyte) or different types of bioactive agents (e.g., antibody, antisense molecule, or small molecule).

In one embodiment, the cells are glial cells that express the NG2 proteoglycan (NG2(+) cells), which are considered to be oligodendrocyte progenitors (OPCs) in the central-nervous system (CNS), based on their ability to give rise to mature oligodendrocytes. In one embodiment, a bioactive agent is administered to a subject to modulate chemokine-signaling mediated by CXCR2, resulting in an increased number of NG2+ OPCs.

In some embodiments, oligodendrocyte progenitor cells (OPC), Schwann cells (SCs), olfactory bulb ensheathing cells, astrocytes, microglia or neural stem cells (NSCs) are cultured, transformed with a vector encoding a chemokine and expanded in vitro prior to transplantation. In other embodiments, the cells may be transfected or genetically modified in vivo to express a protein that inhibits CXC-mediated signaling.

In some embodiments, the myelin producing cells or progenitor cells thereof include but are not limited to fetal or adult OPCs. In one embodiment the OPC is A2B5⁺PSA⁻NCAM⁻ phenotype (positive for the early oligodendrocyte marker A2B5 and negative for polysialylated neural cell adhesion molecule).

Remyelination of CNS axons has been demonstrated in various animal-models. Many recent-studies have since demonstrated new techniques and novel mechanisms associated with the use of cell transplantation in demyelinating disease. Human OPCs isolated from adult brains were able to myelinate naked axons when transplanted into a dysmyelinating mouse mutant. Importantly, the use of adult progenitor cells may avoid ethical concerns. While OP cells are responsible for endogenous remyelination, NSCs are an alternative source of cells to promote myelin repair. NSCs are found in the adult CNS, can be expanded extensively in vitro, and can differentiate to form OLs, astrocytes, or neurons. When transplanted into rodents with relapsing or chronic forms of EAE, NSCs have been shown to migrate to areas of CNS inflammation and demyelination and to preferentially adopt a glial cell-fate. Furthermore, attenuation of clinical disease in transplanted mice have been associated with repair of demyelinating lesions and decreased axonal injury. Histological analysis confirmed that transplanted NSCs differentiated predominantly into PDGFR⁺ OP cells.

Interestingly, while the number of OP cells are increased in NSC-transplanted EAE mice, the majority of these cells are not donor-derived, indicating that the transplanted cells regulated the expansion of endogenous oligodendroglia. The mechanisms by which NSCs promote EAE amelioration and lesion repair are indicative of immunosuppressive and neuroprotective functions. NSC have been demonstrated to induce apoptosis of T cells both in vivo and in vitro, to decrease CNS infiltrating T cells in NSC-transplanted EAE rodents and to inhibit myelin peptide-specific T cell proliferation in vitro. The immunomodulatory and proposed neuroprotective properties may be mediated by neurotrophic and various growth factors which may decrease CNS inflammation and/or enhance OL lineage cell survival and promote remyelination in the host CNS.

In some embodiments, oligodendrocyte progenitor cells (OPC), Schwann cells (SCs), olfactory bulb ensheathing cells, and neural stem cells (NSCs) are transfected with one or more expression vectors, using methods known in the art or disclosed herein, so as to enable expression of one or more desired bioactive agent. Such bioactive agents can be directed to the immunomodulation, by preventing CXC-mediated signaling. In another embodiment, such agents can promote oligodendrocyte proliferation at or migration to lesion sites. In yet another embodiment, such agents can promote OPC differentiation into mature oligodendrocyte, and/or proliferation or migration to lesion sites. In various embodiments, the cells are transfected before, concurrent or subsequent to expansion in culture.

It will be appreciated that transplantation is conducted using methods known in the art, including invasive, surgical, minimally invasive and non-surgical procedures. Depending on the subject, target sites, and agent(s) to be delivered, the type and number of cells can be selected as desired using methods known in the art.

### IV. Screening Assays

### A. Cell Culture

Some aspects of the invention are directed to methods of screening candidate agents to determine if such agents inhibit CXC-mediated cell signaling. Immunomodulatory, myelin repair, or axonal protection inducing agents may be screened in combination to determine which combination is beneficial in treating a neuropathy. In some embodiments, neural cells, particularly glial cells, more particularly, astrocytes, oligodendrocytes, SCs, OPCs or NSCs are cultured and/or genetically modified for screening.

In one embodiment, one or more bioactive agent is placed in contact with such a culture of cells, and before, concurrent or subsequent to such contact, one or more myelin repair- or axonal protection-inducing agent is also administered to the cells, to determine which bioactive agent or combination of bioactive agent produces a desired effect, or a synergistic effect. For example, a synergistic effect may be observed in culture by utilizing time-lapse microscopy revealing a transition from precursor cell types to myelinating oligodendrocyte. Furthermore, progenitor cells can be transfected with a membrane-targeted form of enhanced green fluorescent protein (EGFP) to facilitate convenient fluorescence microscopy in detection of differentiated cells. Therefore, in various embodiments, cells can be cultured and/or genetically modified to express marker proteins or bioactive agents that are components of a combinatorial treatment or screening process utilizing techniques that are known in the art, such as disclosed in U.S. Pat. Nos. 7,008,634; 6,972,195; 6,982,168; 6,962,980; 6,902,881; 6,855,504; or 6,846,625.

In one embodiment, an expression vector can encode a marker protein (e.g., fluorescent marker) that is expressed from a cell-specific promoter element (e.g., PLP or PDGFα, which are specific for glial cells, including oligodendrocyte). Further, the same cells can be transfected with a second expression vector that encodes a CXC chemokine, such as CXCL1 or CXCL8. Alternatively, a single expression construct can encode more than one polypeptide, such as marker protein and a CXC chemokine. In such embodiments, cells expressing a CXC chemokine and a marker protein can be detected using standard microscopy techniques known in the art, including but not limited to fluorescence microscopy (including for in vitro cell or tissue culture or in vivo imaging).

In some embodiments, neural cells are transfected with a nucleic acid molecule that is operably linked to a constitutive, inducible or neural-cell-specific promoter and encodes a chemokine-receptor ligand. Such cells can be transformed to express a CXCL at altered levels that modulating chemokine-mediated signaling. Furthermore, such cells can be administered to an animal subject to enhance neural cell proliferation and/or migration. In one embodiment, cells are genetically modify to provide altered expression of CXCL1, CXCL2, CXCL3, CXCL4, CXCL6, CXCL8 or CXCL10. In one embodiment neural cells are genetically modified to increase expression of CXCL1, CXCR1 or CXCR2. Nucleic acids encoding a desired CXCL can be transformed into target cells by homolgous recombination, integration or by utilization of plasmid or viral vectors utilizing components and methods described herein and familiar to those of ordinary skill in the art. In various embodiments embodiment neural cells are genetically modified to decrease expression of CXCL1, CXCR1, and/or CXCR2.

In should be clear to one of ordinary skill in the art, that expression levels in glial cells can be altered by expression of a desired polypeptide encoded on an expression construct that is administered to such glial cells. Alternatively, expression can be modulated by utilizing expression constructs that encode a product (e.g., antisense molecule, siRNA, aptamer) that itself affects expression of a desired polypeptide, such as CXCL1 or CXCR2. Antisense molecules, siRNA or aptamers can be selected utilizing processes familiar to one of skill in the art, or as described herein above. Other bioactive agents, such as antibodies and small molecules, such as those described above, may also alter expression of a desired polypeptide, such as a polypeptide involved in CXC signaling. CXCR2 and/or CXCR1 signaling may be affected by altering its expression, or expression of its upstream regulators or ligands, or its downstream effectors.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoechste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan probes) resulting in increased specificity and sensitivity. Methods for performing probe-based quantitative amplification are well established in the art and are taught in U.S. Pat. No. 5,210,015.

In yet another aspect, conventional hybridization assays using hybridization probes that share sequence homology with CXC-chemokine related genes can be performed. Typically, probes are allowed to form stable complexes with the target polynucleotides (e.g., CXCL or CXCR genes) contained within the biological sample derived from the test subject in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

As is known to one skilled in the art, hybridization can be performed under conditions of various stringency. Suitable hybridization conditions for the practice of the present invention are such that the recognition interaction between the probe and target CXC related gene is both sufficiently specific and sufficiently stable. Conditions that increase the stringency of a hybridization reaction are widely known and published in the art. See, for example, (Sambrook, et al., (1989), supra; Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present invention include any composition detectable by photochemical, biochemical, spectroscopic, immunochemical, electrical, optical or chemical means. A wide variety of appropriate detectable labels are known in the art, which include fluorescent or chemiluminescent labels, radioactive isotope labels, enzymatic or other ligands. In various embodiments, one will likely desire to employ a fluorescent label or an enzyme tag, such as digoxigenin, β-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphoimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

An agent-induced change in expression CXC chemokine related genes can also be determined by examining the corresponding gene products. Determining the protein level typically involves a) contacting the protein contained in a biological sample comprising myelinating cells with an agent that specifically bind to the CXC chemokine related protein; and (b) identifying any agent:protein complex so formed. In one aspect of this embodiment, the agent that specifically binds a CXC chemokine related protein is an antibody, preferably a monoclonal antibody.

It should be understood that the foregoing compositions and methods are readily adapted to methods described herein below for screening of and treatment with effective amounts of therapeutic agents directed to blocking chemokine signaling (for example, through chemokine receptors or its ligands), resulting in chemokine-mediated immunomodulation and/or enhancement of myelin repair.

An agent-induced change in expression CXC chemokine related genes or an agent-induced effect, may also be determined by detecting marker proteins. For example, marker proteins can be targets for immunostaining techniques known in the art to facilitate identification of cells (e.g., cell fate mapping). Non-limiting exemplary marker proteins of a myelinating cell (including oligodendrocyte and Schwann cell) may be selected from the group consisting of CC1, myelin basic protein (MBP), ceramide galactosyltransferase (CGT), myelin associated glycoprotein (MAG), myelin oligodendrocyte glycoprotein (MOG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), galactocerebroside (GalC), sulfatide and proteolipid protein (PLP). MPZ, PMP22 and P2 are markers for Schwann cells.

If desired, cells (in culture or in vivo) can be modified to express fluorescent marker proteins, for example, so as to follow cell migration in vivo or in tissue culture. Non-exclusive examples of marker genes that can be used in the present invention include reef coral fluorescent proteins (RCFPs), HcRed1, AmCyan1, AsRed2, mRFP1, DsRed1, jellyfish fluorescent protein (FP) variants, red fluorescent protein, green fluorescent protein (GFP), blue fluorescent protein, luciferase, GFP mutant H9, GFP H940, EGFP, tetramethylrhodamine, Lissamine, Texas Red, EBFP, ECFP, EYFP, Citrine, Kaede, Azami Green, Midori Cyan, Kusabira Orange and naphthofluorescein, or enhanced functional variants thereof. Many genes encoding fluorophore proteins markers are known in the art, which markers are capable of use in the present invention. See, website: <cgr.harvard.edu/thornlab/gfps.htm>. Mutated version of fluorescence proteins that emit light of greater intensity or which exhibit wavelength shifts can also be utilized in the compositions and methods of the present invention; such variants are known in the art and commercially available. (See Clontech Catalogue, 2005).

Visualizing fluorescence (e.g., marker gene encoding a fluorescent protein) can be conducted with microscopy techniques, either through examining cell/tissue samples obtained from an animal (e.g., through sectioning and imaging using a confocal microscope), as well as examining living cells or detection of fluorescence in vivo. Visualization techniques include but are not limited utilization of confocal microscopy or photo-optical scanning techniques known in the art. Generally, fluorescence labels with emission wavelengths in the near-infrared are more amenable to deep-tissue imaging because both scattering and autofluorescence, which increase background noise, are reduced as wavelengths are increase. Examples of in vivo imaging are known in the art, such as disclosed by Mansfield et al., J. Biomed. Opt. 10:41207 (2005); Zhang et al., Drug Met. Disp. 31:1054-1064 (2003); Flusberg et al., Nat. Methods 2:941-950 (2005); Mehta et al., Curr. Opin. Neurobiol. 14:617-628 (2004); Jung et al.; J. Neurophysiol. 92:3121-3133 (2004); U.S. Pat. Nos. 6,977,733 and 6,839,586, each disclosure of which is herein incorporated by reference.

The screening assays may also provide a method to screen and identify agents that selectively inhibits a specific CXC chemokine or CXCR mediated signaling pathway. For example, small molecules or antibodies, or other potential CXC related inhibitors may be screened to determine if an agent inhibits or blocks CXCR2 and/or CXCR1 mediating signaling, for example, by inhibiting CXCR2 or its ligands, more effectively than other CXC related protein mediated signaling pathways, such as CXCR1, CXCR3, or CXCR4 mediated signaling. Alternatively, an agent may selectively inhibit CXCR1, whereby the agent is more effective at inhibiting CXCR1, or its pathway, than other CXC related proteins, or their pathways, such as CXCR2, CXCR3, or CXCR4. In some embodiments, an inhibitor may be selective for two CXC receptors, for example, CXCR1 and/or CXCR2. The inhibitor may be more effective at inhibiting CXCR1 and CXCR2, or their signaling pathways, when compared to CXCR3 and/or CXCR4. Inhibitors may directly bind the CXC receptors or its ligands. The inhibitors may directly bind the active sites of the CXC receptors, or the binding sites of the CXC receptors or ligands. Alternatively, the inhibitors may be allosteric inhibitors.

Agents of any type that selectively negatively regulate CXCR2 expression or activity can be used as selective CXCR2 inhibitors in the methods of the invention. Agents of any type that selectively negatively regulate CXCR1 expression or activity can be used as selective CXCR1 inhibitors in the methods of the invention. Moreover, agents of any type that selectively negatively regulate CXCR2 expression or activity, or agents of any type that selectively negatively regulate CXCR1 expression or activity, and that possess acceptable pharmacological properties can be used as selective CXCR2 inhibitors and/or selective CXCR1 inhibitors in the therapeutic methods of the invention.

The relative efficacies of agents as inhibitors of an enzyme activity (or other biological activity) can be established by determining the concentrations at which each agent inhibits the activity to a predefined extent and then comparing the results. In various embodiments, a determination is the concentration that inhibits 50% of the activity in a biochemical assay, i.e., the 50% inhibitory concentration or “IC₅₀”. IC₅₀ determinations can be accomplished using conventional techniques known in the art. In general, an IC₅₀ can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the IC₅₀ value. Analogously, other inhibitory concentrations can be defined through appropriate determinations of activity. For example, in some settings it can be desirable to establish a 90% inhibitory concentration, i.e., IC₉₀, etc.

Accordingly, a selective CXCR2 inhibitor, or an inhibitor that selectively inhibits CXCR2 mediated signaling, alternatively can be understood to refer to a compound that exhibits a 50% inhibitory concentration (IC₅₀) with respect to CXCR2, that is at least at least 10-fold, preferably at least 20-fold, and more preferably at least 30-fold, lower than the IC₅₀ value with respect to any or all of the other CXCR family members. Accordingly, a selective CXCR1 inhibitor, or an inhibitor that selectively inhibits CXCR2 mediated signaling, alternatively can be understood to refer to a compound that exhibits a 50% inhibitory concentration (IC₅₀) with respect to CXCR1, that is at least at least 10-fold, preferably at least 20-fold, and more preferably at least 30-fold, lower than the IC₅₀ value with respect to any or all of the other CXCR family members.

CXCR1 and/or CXCR2 activity may be determined by binding assays, for example, determining the IC₅₀ of an inhibitor that selectively displaces CXCL8 binding to CXCR2. CXCR1 or CXCR2 activity may also be determined by the amount of recruitment of neutrophil polymorphonuclear leukocytes (PMNs) as compared to control cells. CXCL8, a ligand for both CXCR1 and CXCR2, promotes PMN recruitment. PMN adhesion assays, PMN activations assays, and T cell chemotaxis assays may be performed to determine the effect of a CXCR1 or CXCR2 inhibitor (Castilli et al., Biochem Pharma. 69:385-395 (2005)). Selective CXCR1 or CXCR2 inhibition may also be determined by expression levels of the genes (for example by RT-PCR) or expression levels of the proteins (for example by immunocytochemistry, immunohistochemistry, Western blots) as compared to other CXCRs.

In another aspect, the present invention provides screening of bioactive agents effective in ameliorating gliosis. Astrocytes may activate themselves and microglia, and induce gliosis, in demyelinating conditions. Identification of hypertrophic reactive astrocytes in cells treated with a bioactive agent as compared to a control may be used to determine if a bioactive agent is effective in ameliorating gliosis. Cultures of spinal cord astrocytes may be assayed by immunocytochemistry and dot blot assays to analyze secreted and bound chondroitin sulphate proteoglycan (CSPG). Expression of glial fibraillary acidic protein (GFAP), as well as other intermediate filament proteins, such as vimentin, and other proteoglycans produced by astrocytes, such as heparan sulphate proteoglycan (HSPG), dermatan sulphate proteoglycan (DSPG), or keratan sulphate proteoglycan (KSPG), and chondroitin sulphate proteoglycan (CSPG) may also be detected.

Biologically active agents effective in promoting migration of glial cells may also be determined. For example, migration of OPCs to lesion sites may be determined by generating demyelinating lesions in adult rats through localized injection of LPC into the dorsal columns. Purified labeled OPCs may be injected into the slice adjacent to, or into the lesion area, and their behavior monitored, thus following cell migration in this system. Control and treated cultures may be maintained to assay the level of remyelination in the lesion(s). Multiple assays may be done on a single lesioned animal since each slice is independent. The post lesion interval and dose at which a CXC-signaling inhibitor (e.g., repertaxin) is most effective may be determined thereby facilitating in vivo studies.

### B. Animal Models

In some aspects, screening assays for determining a beneficial therapeutically effective combination of bioactive agents directed to immunomodulation and myelin repair/remyelination or axonal protection are conducted utilizing animal models. In some embodiments, an animal is a small rodent, or simian species. In further embodiments, an animal is a mouse, rat, guinea pig, or monkey.

In some embodiments, the animal is a transgenic animal that can be a “knock-out” or “knock-in”, with one or more desired characteristics. For example, in some embodiments, a transgenic animal can be modified to express or express at altered levels (i.e., up or down) an agent that promotes immunomodulation, myelin repair/remyelination or axonal protection. Therefore, such an animal is utilized to screen a plurality of different bioactive agents also directed to immunomodulation, myelin repair/remyelination or axonal protection, where if the transgenic animal comprises an agent directed to one end point, then the animal is administered an agent directed to a different end point(s), and vice versa, to identify a candidate combination of therapeutic agents that result in a synergistic therapeutic result for a neuropathy or related conditions described herein above.

As noted above, transgenic animals can be broadly categorized into two types: “knockouts” and “knockins”. A “knockout” has an alteration in the target gene via the introduction of transgenic sequences that results in a decrease of function of the target gene, preferably such that target gene expression is insignificant or undetectable. A “knockin” is a transgenic animal having an alteration in a host cell genome that results in an augmented expression of a target gene, e.g., by introduction of an additional copy of the target gene, or by operatively inserting a regulatory sequence that provides for enhanced expression of an endogenous copy of the target gene. The knock-in or knock-out transgenic animals can be heterozygous or homozygous with respect to the target genes. Both knockouts and knockins can be “bigenic”. Bigenic animals have at least two host cell genes being altered. In one embodiment, a bigenic animal carries a transgene encoding a neural cell-specific recombinase and another transgenic sequence that encodes neural cell-specific marker genes.

In other embodiments, the transgenic model system can also be used for the development of a bioactive agent that promote or are beneficial for a neuronal remyelination. For example, a transgenic animal that is modified to express an agent resulting in an immunomodulatory, myelin repair or axonal protection phenotype, can be utilized in methods of screening unknown compounds to determine (1) if a compound enhances immune tolerance, suppresses an inflammatory response, or promotes remyelination and/or (2) if a compound can result in a synergistic therapeutic effect in the animal model. Moreover, neural cells can be isolated from the transgenic animals of the invention for further study or assays conducted in a cell-based or cell culture setting, including ex vivo techniques. Furthermore, the model system can be utilized to assay whether a test agent impart a detrimental effect or reduces remyelination, e.g., post demyelination insult.

Advances in technologies for embryo micromanipulation now permit introduction of heterologous DNA into fertilized mammalian ova as well. For instance, totipotent or pluripotent stem cells can be transformed by microinjection, calcium phosphate mediated precipitation, liposome fusion, retroviral infection or other means. The transformed cells are then introduced into the embryo, and the embryo will then develop into a transgenic animal. In one embodiment, developing embryos are infected with a viral vector containing a desired transgene so that the transgenic animals expressing the transgene can be produced from the infected embryo. In another embodiment, a desired transgene is coinjected into the pronucleus or cytoplasm of the embryo, preferably at the single cell stage, and the embryo is allowed to develop into a mature transgenic animal. These and other variant methods for generating transgenic animals are well established in the art and hence are not detailed herein. See, for example, U.S. Pat. Nos. 5,175,385 and 5,175,384.

Accordingly, in some embodiments the present invention provides a method of using animal models for detecting and quantifying synergistic, or increased synergistic, combinatorial treatment. In one embodiment, the method comprises the steps of: (a) inducing demyelination insult in the transgenic animal of the invention expressing an immunotolerance-inducing agent; (b) administering a candidate agent and an allowing time for myelin repair occur if it is to occur; (c) detecting and/or quantifying expression of cell-specific marker gene(s) (d) determining if and how much remyelination has occurred and if such remyelination is enhanced as compared to a control. In such an example, the control could be wild-type in which a disease model is induced, or a transgenic to which the candidate agent is not administered.

A number of methods for inducing demyelination in a test animal have been established. For instance, neuronal demyelination may be inflicted by pathogens or physical injuries, agents that induce inflammation and/or autoimmune responses in the test animal. In various embodiments, a method of the invention employs demyelination-induced agents including but not limited to IFN-γ and cuprizone (bis-cyclohexanone oxaldihydrazone). The cuprizone-induced demyelination model is described in Matsushima et al., (Brain Pathol. 11:107-116 (2001)). In this method, the test animals are typically fed with a diet containing cuprizone for a few weeks ranging from about 1 to about 10 weeks.

After induction of a demyelination condition by an appropriate method, the animal is allowed to recover for a sufficient amount of time to allow remyelination at or near the previously demyelinated lesions. While the amount of time required for developing remyelinated axons varies among different animals, it generally requires at least about 1 week, more often requires at least about 2 to 10 weeks, and even more often requires about 4 to about 10 weeks. Remyelination can be ascertained by observing an increase in myelinated axons in the nervous systems (e.g., in the central or peripheral nervous system), or by detecting an increase in the levels of marker proteins of a myelinating cell.

Animals may also be administered prior, concurrent, or subsequent to a demyelination a bioactive agent. Amount of gliosis may be determined and compared between a control animal with an animal treated with a biologically active agent. Bioactive agents effective in ameliorating gliosis may be ascertained. Gliosis may contribute to glial scars, typically a barrier to remyelination and axonal regeneration. Gliosis may be induced by reactive astrocytes. Astrocyte activation and gliosis may contribute to demyelination, as the presence of astrogliosis and microgliosis is a feature typical of faulty demyelination and neuroinflammatory diseases. Identification of hypertrophic reactive astrocytes may be determined by immunocytochemical methods. Expression of glial fibraillary acidic protein (GFAP), as well as other intermediate filament proteins, such as vimentin, as well as proteoglycans produced by astrocytes, such as heparan sulphate proteoglycan (HSPG), dermatan sulphate proteoglycan (DSPG), keratan sulphate proteoglycan (KSPG), and chondroitin sulphate proteoglycan (CSPG) may also be detected to identify gliosis. The expression of GFAP, vimentin, HSPG, DSPG, KSPG, and/or CSPG may be compared between cells treated with or without a bioactive agent to determine if an agent is effective in ameliorating gliosis.

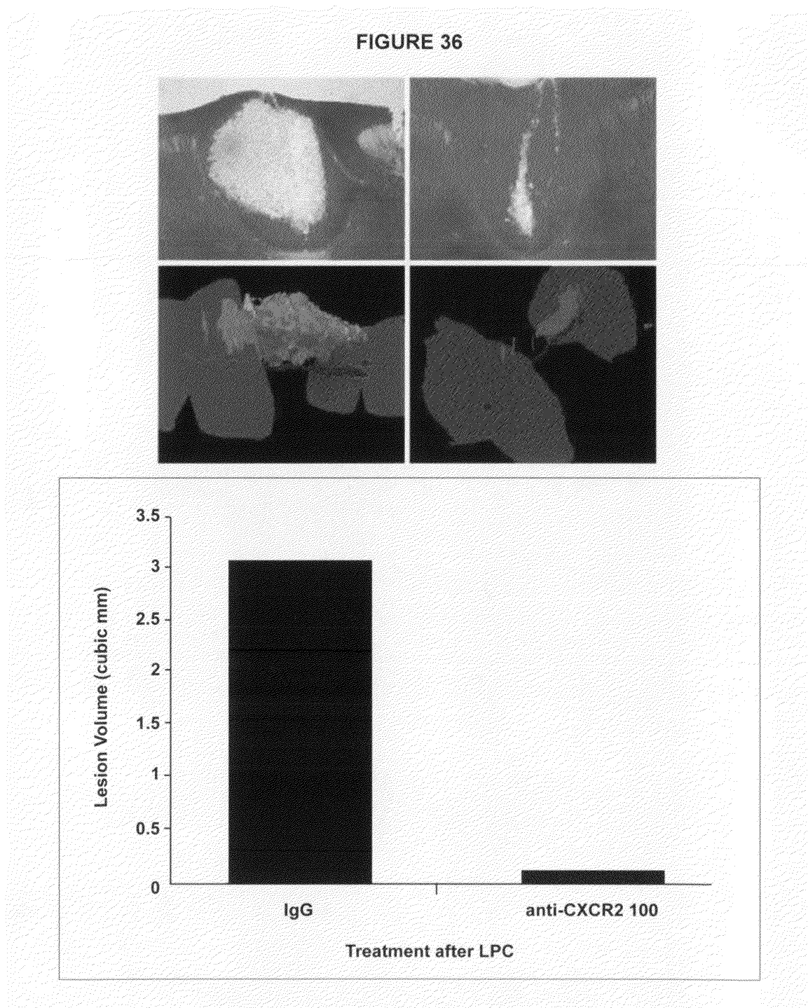

Biologically active agents that selectively inhibit CXCR2 and/or CXCR1 may be administered to animals. Levels of CXCR2 and/or CXCR1 proteins may be assayed and compared to glial fibrillary acidic protein (GFAP) expression in the injury area. Spinal cords may be sectioned and processed for immunohistochemistry or histology. Entire lesion sections may be obtained and stained with Luxol fast blue, a stain for myelin (FIG. 36). The lesion may be reconstructed using 3D Doctor software program where lesion volume and 3 dimensional images can be constructed (FIG. 36). Protein expression within the lesion may be analyzed using standard immunohistochemistry techniques using antibodies for myelin basic protein (MBP), GFAP, ED1 (macrophage/microglia), and vimentin.

### V. Therapeutics

### A. Dosage

Depending on the patient and condition being treated and on the administration route, the bioactive agents disclosed herein (e.g., small molecules, repertaxin or peptides/polypeptides) targeting CXC-mediated signaling will generally be administered in dosages of 0.01 mg to 500 mg V/kg body weight per day, e.g. about 20 mg/day for an average person. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. Thus for example oral dosages in the rat may be ten times the injection dose. A typical dosage may be one injection daily or multiple injections daily. In various embodiments, such agents are administered and are designed to block or inhibit CXCR1 and/or CXCR2. In one embodiment, one or more agents are administered to block or inhibit CXCR1, CXCR2, CXCL1, CXCL5, CXCL8 or a combination thereof.

Those of skill will readily appreciate that dose levels can vary as a function of the specific compound, the severity of the symptoms and the susceptibility of the subject to side effects. Some of the specific peptides are more potent than others. In some embodiments, dosages for a given complex are readily determinable by those of skill in the art by a variety of means. For example, one means is to measure the physiological potency of a given compound.

In some embodiments, peptides, polypeptides or antibodies can be administered at dosages determined to be therapeutic. In some embodiments, within one or more combinatorial method of the invention the immunoregulatory component comprises peptides or polypeptides, including but not limited to antibodies, peptides, proteins, aptamers, siRNA, small molecules or antisense at dosages of 0.01 mg to 500 mg V/kg body weight per day. In some embodiments, such agents are administered from between 3 to 5, 4 to 6, 5 to 7, or 6 to 10 times a day at the same or varying dosages. In some embodiments, the administration is repeated in a plurality of cycles, where each cycle comprises administration of an agent between 3 to 5, 4 to 7, 6 to 9, 7 to 10, 8 to 12, 9 to 16 or 10 to 21 days.

In some embodiments, antibodies targeting CXC-mediated signaling are administered at dosages depending upon such factors as the patient's age, weight, height, sex, general medical condition and previous medical history. Typically, it is desirable to provide the recipient with a dosage of antibody component, immunoconjugate or fusion protein which is in the range of from about 1 pg/kg to 10 mg/kg (amount of agent/body weight of patient), although a lower or higher dosage also may be administered as circumstances dictate. Administration of antibodies (or any bioactive agents described herein) to a patient can be intravenous, intraarterial, intraperitoneal, intramuscular, subcutaneous, intrapleural, intrathecal, by perfusion through a regional catheter, or by direct intralesional injection. When administering therapeutic proteins by injection, the administration may be by continuous infusion or by single or multiple boluses. Intravenous injection provides a useful mode of administration due to the thoroughness of the circulation in rapidly distributing antibodies. Such antibodies can be specific for any chemokines disclosed herein. In various embodiments, such antibodies are specific for CXCR1, CXCR2, CXCR3, CXCL1, CXCL2, CXCL5, CXCL8 or IL-8, whereby in a method of treating a neuropathy, such antibodies are administered to treat a subject in need thereof. In some embodiments, two or more antibodies targeting CXCRs and/or CXCLs can be administered or contacted to cells expressing said CXCRs and/or CXCLs. In one embodiment, one or more antibodies are administered to block or inhibit CXCR1, CXCR2, CXCL1, CXCL5, CXCL8 or a combination thereof.

In other embodiments, the concentration of the therapeutically active antibody or antibody fragment (e.g., Fab or Fc portion) in a formulation may vary from about 0.1 to 100 weight %. In one embodiment, the concentration of the antibody or antibody fragment is in the range of 0.003 to 1.0 molar. In order to treat a patient, a therapeutically effective dose of the antibody or antibody fragment may be administered. By “therapeutically effective dose” herein is meant a dose that produces the effects for which it is administered (e.g., blocking co-stimulation of T cells or B cells). The exact dose will depend on the purpose of the treatment, and will be ascertainable by one skilled in the art using known techniques. Dosages may range from 0.01 to 100 mg/kg of body weight or greater, for example 0.1, 1, 10, or 50 mg/kg of body weight, with 1 to 10 mg/kg being preferred. As is known in the art, adjustments for antibody or Fc fusion degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art. Such antibodies can be specific for any chemokines disclosed herein. In various embodiments, such antibodies are specific for CXCR1, CXCR2, CXCR3, CXCL1, CXCL2, CXCL5, CXCL8 or IL-8. In some embodiments, one or more antibodies are administered and each is specific for CXCR1 and CXCR2.

Administration of the pharmaceutical composition comprising an antibody or antibody fragment, preferably in the form of a sterile aqueous solution, may be done in a variety of ways, including, but not limited to orally, subcutaneously, intravenously, intranasally, intraotically, transdermally, topically (e.g., gels, salves, lotions, creams, etc.), intraperitoneally, intramuscularly, intrapulmonary (e.g., AERx® inhalable technology commercially available from Aradigm, or Inhance™ pulmonary delivery system commercially available from Inhale Therapeutics), vaginally, parenterally, rectally, or intraocularly. In some instances, for example for the treatment of wounds, inflammation, etc., the antibody or Fc fusion may be directly applied as a solution or spray. As is known in the art, the pharmaceutical composition may be formulated accordingly depending upon the manner of introduction. In various embodiments, the antibodies are administered at low protein doses, such as 20 milligrams to 2 grams protein per dose, given once, or repeatedly, parenterally. Alternatively, antibodies are administered in doses of 20 to 1000 milligrams protein per dose, or 20 to 500 milligrams protein per dose, or 20 to 100 milligrams protein per dose. In some embodiments, such agents are administered from between 3 to 5, 4 to 7, 6 to 9, 7 to 10, 8 to 12, 9 to 16 or 10 to 21 days. In some embodiments, the administration is repeated in a plurality of cycles, where each cycle comprises administration of an agent between 3 to 5, 4 to 7, 6 to 9, 7 to 10, 8 to 12, 9 to 16 or 10 to 21 days.

The antibodies, alone or conjugated to liposomes, can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, REMINGTON'S PHARMACEUTICAL SCIENCES, 19th Ed. (1995).

For purposes of therapy, antibodies are administered to a patient in a therapeutically effective amount in a pharmaceutically acceptable carrier. In this regard, a “therapeutically effective amount” is one that is physiologically significant. An agent is physiologically significant if its presence results in a detectable change in the physiology of a recipient patient. In the present context, an agent is physiologically significant if its presence results in blocking immune cell activation, proliferation or differentiation. In some embodiments, the immune cells are T cells or B cells.

Additional pharmaceutical methods may be employed to control the duration of action of an antibody in a therapeutic application. Control release preparations can be prepared through the use of polymers to complex or adsorb the antibody. For example, biocompatible polymers include matrices of poly(ethylene-co-vinyl acetate) and matrices of a polyanhydride copolymer of a stearic acid dimer and sebacic acid. (Sherwood et al., Biotechnology 10:1446-1449 (1992)). The rate of release of an antibody from such a matrix depends upon the molecular weight of the protein, the amount of antibody within the matrix, and the size of dispersed particles. (Saltzman and Langer, Biophys. J 55:163-171 (1989); Sherwood et al., supra). Other solid dosage forms are described in REMINGTON'S PHARMACEUTICAL SCIENCES, 19th ed. (1995).

### B. Pharmaceutical Compositions

Pharmaceutical compositions is contemplated where a is formulated for pharmaceutical use. Such compositions can comprise antagonists for any chemokines disclosed herein. In some embodiments, such compositions are antagonists for CXCR1, CXCR2, CXCR3, CXCL1, CXCL2, CXCL5, CXCL8 or IL-8. In some embodiments, such compositions inhibit or block CXCR1, CXCR2, CXCL1, CXCL5, CXCL8 or a combination thereof. In one embodiment, one or more compositions inhibit or block CXCR1 and/or CXCR2.

Formulations of such agents are prepared for storage by mixing such agents having the desired degree of purity with optional pharmaceutically acceptable carriers, excipients or stabilizers (Remington's Pharmaceutical Sciences 16th edition, Osol, A. Ed., 1980), in the form of lyophilized formulations or aqueous solutions. Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In some embodiment, the pharmaceutical composition that comprises the bioactive agents of the present invention is in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. For example, the ammonium, potassium, sodium, calcium, and magnesium salts may be used. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferrably sterile. T his is readily accomplished by filtration through sterile filtration membranes or other methods known in the art.

The agents targeting CXCR-signaling may also be formulated as immunoliposomes. A liposome is a small vesicle comprising various types of lipids, phospholipids and/or surfactant that is useful for delivery of a therapeutic agent to a mammal. Liposomes containing bioactive agents are prepared by methods known in the art, such as described in Eppstein et al., Proc. Natl. Acad. Sci. USA 82:3688-3692 (1985); Hwang et al., Proc. Natl. Acad. Sci. USA 77:4030-4034 (1990); U.S. Pat. Nos. 4,485,045; 4,544,545; and PCT WO 97/38731. Liposomes with enhanced circulation time are disclosed in U.S. Pat. No. 5,013,556. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al., J. National Cancer Inst 81:1484-1488 (1989).

The subject agents can also be formulated to yield a controlled-release formulation.

### EXAMPLES

### Example 1

### Experimental Animals

All mice were kept in micro-isolation in a pathogen-free environment in the Animal Resource Center of Case Western Reserve University and all procedures were conducted according to approved IAUC guidelines. Mice lacking the CXCR2 receptor (BALB/c-Cmkar2^{tmlMwm}) and matching wild-type (WT) strains were purchased from The Jackson Laboratories (Bar Harbor, Me.). The Cxcr2^{-/-} mice contain a targeted mutation in the mIL-8Rh/Cxcr2 gene which was introduced to this background from a C129S2(B6)-Cmkar2tmlMwm donor strain (Cacalano et al., Science 265:682-684 (1994)). The PLP/DM20-EGFP (C57BL/6) Taconic mice were provided by Dr. Wendy Macklin (Mallon et al., J. Neurosci. 22:876-885 (2002)). Both mouse strains were independently expanded by inbreeding into colonies from which mice were removed for crossing to generate Cxcr2^{+/-}:PLP/DM20-EGFP+ mice. The mixed strain colonies were expanded and maintained by sibling mating of Cxcr2^{+/-}:PLP/DM20-EGFP+ mice to generate Cxcr2^{+/+}:PLP/DM20-EGFP+ and Cxcr2^{-/-}:PLP/DM20-EGFP+ siblings. All experiments were done using sex matched Cxcr2^{+/+} and Cxcr2−/− littermates from at least 5 crosses. The Cxcr2 genotype was confirmed by PCR of DNA isolated from tail digestions in order to identify WT and targeted (KO) Cxcr2 alleles. Specific primers for the Cxcr2 gene were IMR453 (5-GGTCGTACTGCGTATCCTGCCTCAG-3; SEQ ID NO: 1) and IMR454 (5-TACCATGATCTTGAGAAGTCCATG-3; SEQ ID NO: 2) and for the inserted neomycin gene in the Cxcr2^{-/-} mice, JMR013 (5-CTTGGGTGGAGAGGCTATTC-3; SEQ ID NO: 3), and JMR014 (5-AGGTGAGATGACAGGAGATC-3; SEQ ID NO: 4) (Tsai et al., Cell 110:373-383 (2002)). The PCR products were separated on 1.2% agarose gels. Wild-type animals exhibit a single band of 360-bp, Cxcr2^{-/-} mice exhibit a shorter 280-bp band, and heterozygote mice exhibit both. To evaluate expression of the PLP/DM20-EGFP construct, clips of mice tails were visualized directly by fluorescent microscopy.

Induction of Demyelination with Cuprizone

Cxcr2^{-/-}:PLP/DM20-EGFP⁺ and Cxcr2^{-/-}:PLP/DM20-EGFP⁺ 8-10 week old mice were put on a 0.2% wt/wt cuprizone (Bis-cyclohexanone-oxaldihydrazone, C9012, Sigma, St. Louis Mo.) milled chow diet. Control mice were fed a regular diet without cuprizone. A maximum of three mice were housed together per cage and mice were segregated by sex. The diet was continuously supplied to the mice until the time of sacrifice which was either 3-4 weeks, or 6-7 weeks, after treatment initiation. The mice were monitored every other day for signs of weakness or distress and any mice which appeared to be severely affected due to toxicity were euthanized.

Induction of Demyelination with Lysolecithin

Both BALB/c and BALB/c:C57BL/6 Cxcr2^{+/+} and Cxcr2^{-/-} mice were used for these experiments. 2-3 month old mice were anesthetized by intraperitoneal injection of a cocktail containing ketamine hydrochloride, xylazine hydrochloride, and acepromazine. Once the animals were under the effect of the anesthesia, they were weighed, their backs were shaved, and O.O1 mL of turbogesic was injected subcutaneously in their backs. The animals were subsequently stabilized in a stereotaxic surgery base plate with a Cunningham mouse spinal adaptor (#51690, Stoelting Co., Wood Dale, Ill.) using transverse clamps (#51691, Stoelting Co., Wood Dale, Ill.). The vertebral column was exposed and a segment of the dorsal spinal cord was visible without the need for dorsal laminectomy, due to the position that the mice adopt under this instrument. A borosilicate pulled glass micropipette filled with a 1 μg/μL μsolution of lysophosphatidyl choline (Lysolecithin or LPC; L4129, Sigma, St. Louis, Mo.) attached to a PHD200 pump (Harvard) was slowly directed to the side of the visible dorsal vessel of the spinal cord. The needle was slowly inserted past the dura into the dorsal spinal cord and retracted slightly before injection of 1 uL of 1% LPC to the dorsal column at a rate of 0.25 μL per minute. The needle was allowed to rest in place for 1-2 minutes after the fluid was injected to prevent backflow. A suture was applied to the soft tissue adjacent to the injection site to facilitate its future identification. Animals were left to recover for 7 to 14 days at which times they were sacrificed for evaluation.

### Example 2

### Tissue Preparation

For histological analyses, mice were anesthetized with an intraperitoneal injection of ketamine hydrochloride, xylazine hydrochloride and acepromazine and subsequently perfused with heparin (Sigma, St. Louis Mo.) in 0.1M phosphate buffer (0.1M PB) followed by 4% paraformaldehyde (Electron Microscopy Sciences, Hatfield Pa.) in 0.1M PB (4% PFA). The vertebral columns and skulls were removed and postfixed in 4% PFA overnight. Brains and spinal cords were cryoprotected in 30% sucrose (Sigma, St. Louis Mo.) in 0.1M PB. Matching Cxcr2^{+/+} and Cxcr2^{-/-} spinal cords or brains were embedded side by side or independently before sectioning at 20 um using a cryostat (Microm, Heidelberg). Sections were collected serially on glass slides, dried overnight, and stored at −20° until analysis. For electron micrographic analyses, anesthetized animals were perfused initially with a heparin solution and subsequently with a 3% Formaldehyde/3% Glutaraldehyde in 0.1M Sodium Cacodylate buffer Ph=7.4 (Electron Microscopy Sciences, Hatfield Pa.). The tissue was dissected and post-fixed in 1% OsO4. Specimens were dehydrated through a graded series of ethanol dilutions and embedded in Poly/Bed812 resin (Polysciences Inc, Warrington, Pa.). Thick (1 μm) sections were stained with toluidine blue and appropriate areas selected for further sectioning. Ultra-thin sections (100 nm) from matching areas in Cxcr2^{-/-} and Cxcr2^{+/+} tissue blocks were cut and placed on meshed-nickel/cooper grids. The grids were washed repeatedly and double-stained with uranyl acetate and lead citrate and visualized using an electron microscope (JEOL 1200CX) at 80 kV.

### Immunohistochemistry

Slides containing sections from identical spinal cord and brain regions were subjected to immunohistochemistry for myelin basic protein (MBP, myelin), glial fibrillary acidic protein (GFAP, astrocytes), the chondroitin sulfate proteoglycan NG2 (OPCs), and the calcium binding protein IBA-1 (microglia). For MBP stains, slides were thawed and rinsed in PBS 3 times for 10 minutes, submerged in 100% ethanol for 10 minutes, rinsed, and soaked with a 0.5% Triton-X-100 in PBS (PBST) solution for 1 hour before adding primary antibody. Rabbit anti-MBP antibody (1:100, Accurate, BMDV2017, Westbury, N.Y.) diluted in a solution containing 10% NGS (normal goat serum) and 0.5% PBST was added to the samples in a humidified chamber, and left overnight at room temperature. For GFAP stain, slides were thawed and rinsed in PBS 3 times for 10 minutes after which rabbit anti-GFAP antibody (1:50, DAKO Z0334, Carpinteria, Calif.) diluted in a solution containing 10% NGS/1% PBST was added to the samples and left overnight in a humidified chamber at room temperature. On the following day, anti-MBP and anti-GFAP treated slides Were rinsed 6 times in PBS for 10 minutes before addition of the secondary antibody goat anti-rabbit Alexa 546 (1:400, Molecular Probes/Invitrogen A11010, Carlsbad, Calif.) diluted in a solution containing 10% NGS/0.5% PBST for 2 hours at room temperature in a humidified chamber. The slides were then rinsed 6 times in PBS, counterstained with 4′,6-Diamidino-2-phenylindole (DAPI, D1306, Molecular Probes/Invitrogen, Carlsbad, Calif.) and mounted with citifluor mounting media.

For NG2 AND IBA immunohistochemical analyses of CNS tissues, slites were thawed and rinsed in 0.2% PBST, incubated with 0.3% hydrogen peroxide/10% Triton X-100, and blocked with 10% goat serum/0.2% PBST at room temperature for 1 hour. For IBA-1 stains, mouse monoclonal IBA-1 antibody (1:1000, gift from Dr. Bruce Trapp-CCF, Cleveland Ohio) diluted in 1% BSA/0.2% PBST was applied overnight at 4° C. in a humidified chamber. For NG2 stains, sections were incubated for 4 days at 4° C. with anti-NG2 chondroitin sulfate proteoglycan antibody (AB5320, Chemicon Temecula, Calif.). Following the primary antibody step, slides were rinsed and incubated with appropriate secondary antibodies. For NG2, slides were incubated with biotinylated goat anti-rabbit-1:1000 (BA-1000, Vector Laboratories, Burlingame, Calif.), and then with ABC-1:1000 (PK-6100, Vector Laboratories, Burlingame, Calif.) diluted in 1% BSA/0.2% PBST. Sections were developed with DAB (3,3′-diaminobenzidine)/hydrogen peroxide for 5 minutes at room temperature, rinsed in ddHkO, dehydrated, and mounted. For IBA-1 stains, goat anti-mouse Alexa 546 (1:600, Molecular Probes/Invitrogen A11030, Carlsbad, Calif.) diluted in 1% BSA/0.2% PBST was added for 2 hours at room temperature. The slides were then rinsed 6 times in PBS, counterstained with DAPI (D1306, Molecular Probes/Invitrogen, Carlsbad, Calif.), and mounted with citifluor mounting media.

Slides containing sections of Cxcr2^{-/-}:PLP/DM20-EGFP⁺ and Gtcr2^{+/+}:PLP/DM20-EGFP⁺ mice were subjected to immunohistochemistry for CXCR2. These slides were thawed, rinsed in PBS 3 times for 10 minutes, soaked with 5% acid methanol at −20° C. for ten minutes, rinsed, and blocked with 20% NGS/PBS (without triton) for 30 minutes at room temperature. Mouse anti CXCR2 antibody (1:100, Biosource/Invitrogen, Carlsbad, Calif.) was diluted in block and added overnight at 4° C. After rinsing, goat anti-mouse IgG Alexa 488 (1:200, Molecular Probes/Invitrogen A21121, Carlsbad, Calif.) diluted in block was added for 1 hour at room temperature. The slides were then rinsed, counterstained with DAPI and mounted with citifluor mounting media. Cxcr2^{-/-} tissue was used as a negative control.

### Example 3

### Quantification of PLP/EGFP+ and NG2+ Cell Density

To compare the density of oligodendrocyte lineage cells, matching sections from defined areas of the brain and spinal cord from Cxcr2^{+/+}:PLP/DM20-EGFP+ and Cxcr2^{-/-}:PLP/DM20-EGFP+ sex matched siblings were selected and PLP-EGFP+ cells visualized by their autoflourescence. For NG2 histological and immunohistochemical analysis of CNS tissues, 30 cm free floating sections were prepared from mouse spinal cords isolated as previously described. The sections were rinsed in PBST (0.2% TritonX-100 in PBS), incubated with 0.3% hydrogen peroxide in 10% Triton X-100, and blocked with 10% goat serum in PBST at room temperature for 1 hr. These were then incubated for 4 days at 4° C. with anti-NG2 chondroitin sulfate proteoglycan antibody (AB5320, Chemicon Temecula, Calif.). On day 5, tissues were incubated with appropriate biotinylated goat anti-rat-1:1000 (BA-9400, Vector Laboratories, Burlingame, Calif.), then with ABC-1:1000 (PK-6100, Vector Laboratories, Burlingame, Calif.). Sections were washed thrice with PBST after each incubation step past the addition of primary antibody. All antibodies, as well as ABC, were diluted in 1% BSA in PBST. Sections were developed with DAB/hydrogen peroxide for 5 minutes at room temperature, rinsed in ddH₂O, dehydrated, and mounted. Texas Red goat-anti-rat 1:1000 (TI-9400, Vector laboratories, Burlingame, Calif.) was used for immunofluorescence staining, and pre-immune IgG (Sigma-Aldrich, Munich, Germany) was used as negative control. Sections were subsequently mounted on glass slides for analysis.

A fluorescent microscope (Leica DMR) was used to select and record matching CNS regions from NG2 stained or PLP/DM20-EGFP+ autofluorescent slides. The images were adjusted to similar intensity levels and the total number of EGFP+ cells, representing cells of the oligodendrocyte lineage at different stages, and NG2+ cells, representing OPCs, were counted using Image-Pro Express software over defined areas. All counts were performed by an observed blinded to the animal phenotype and divided by the area of the field counted to determine a density. At least 3 different animals of each phenotype were compared in each region. The data was pooled for each CNS region and presented as mean+/−two standard errors of the mean.

### Example 4

### Determination of Relative White Matter Area

Slides containing side-by-side sections of spinal cord from Cxcr2^{-/-}:PLP/DM20-EGFP+ and WT sex-matched sibling littermates were observed under the fluorescent microscope (Leica DMR) and images recorded with a CCD camera (Hamamatsu) using OpenLab Software. The images were analyzed using Image-Pro Express software with which measurements of total spinal cord area and gray matter area were made. The boundaries between gray and white matter, and between white matter and spinal cord edge, were determined by the margin of intense fluorescence and traced to obtain gray matter and total spinal cord areas. White matter area values (calculated by subtracting gray matter from total spinal cord area) were divided by the total values to obtain a ratio of white matter to total spinal cord area. At least 6 sections from each animal were measured as described above and from these an average ratio of white matter to total spinal cord area was calculated for matching cord levels from 10 Cxcr2^{+/+} and 10 Cxcr2^{-/-} mice. Results for separate ages are individually presented as means+/− standard deviation. These were further pooled and presented as mean+/−two standard errors of the mean.

### Example 5

### Ultrastructural Analysis of Myelin Thickness and Nodes of Ranvier

EM images of matching regions of dorsal and ventral spinal cord were scanned using a Hewlett Packard ScanJet5300C scanner and saved using Adobe Photoshop Software. Negative images were inverted and analyzed using Image-Pro Express software by manual tracings of both axon perimeter and myelin thickness for randomly selected myelinated axons in the dorsal columns (gracilis and cuneatus fasciculi, excluding the corticospinal tracts). Three separate measurements of the radial thickness of myelin were taken from each axon and averaged to provide mean myelin thickness. This value was divided by the axon perimeter measurement to obtain a ratio of myelin thickness to axonal perimeter. Five Cxcr2^{-/-} and 6 Cxcr2^{+/+} mice were analyzed and a range of different axon sizes were evaluated to determine mean values of myelin thickness to axonal perimeter ratios. This is not a true G-ratio, which is defined as the ratio of axon diameter over fiber diameter, but is an equivalent measure and will be referred to as a pseudo-G-ratio throughout the paper. Results for separate ages are presented as mean+/−two standard errors of the mean. Dorsal and ventral spinal cord longitudinal sections were analyzed for nodal structure and organization.

### Example 6

### Spinally Elicited Evoked Potentials and Central Conduction Velocity

Recordings were performed using Nicolet Viking IV equipment (Nicolet Biomedical, Madison Wis.). Animals were anesthetized as described above. The lumbar region of the spinal cord was stimulated at a frequency of 4.7 Hz using disposable EEG SS-subdermal needles of a diameter of 0.4 mm. For each animal the stimulus intensity was set by the appearance of bilateral repetitive leg twitches. The spinally elicited evoked potentials were recorded above the skull at two positions equidistant from and perpendicular to the midline. Latencies from stimulus to recording electrode and amplitudes were recorded from at least three trials per animal yielding reproducible waveforms. The distance between the stimulating and recording electrodes was measured in centimeters and divided by the latency in milliseconds to calculate a mean central conduction velocity for each mouse. A total of 10 Cxcr2^{-/-} and 12 Cxcr2^{+/+} mice were analyzed. The conduction velocity results were segregated by genotype and genetic background and plotted as mean+/−standard deviation.

### Somatosensory Evoked Potentials (SSEPs) and Compound (CNS and PNS) Conduction Velocity

Recordings were performed using Nicolet Viking IV equipment (Nicolet Biomedical, Madison Wis.). The tibial region of the hind limb of anesthetized mice was stimulated at a frequency of 3.1 Hz, recordings made in the thoracic region of the spinal cord, and latency and amplitude determined. Three recordings were made for each animal to determine a mean latency and conduction velocity. A total of 7 Cxcr2^{-/-} and 6 Cxcr2^{+/+} mice were evaluated to determine mean conduction velocities.

### Example 7

### Western Blots

Brains and spinal cords from p13 Cxcr2^{-/-} and WT sex matched littermates were flash frozen and stored at −80° C. until needed. Tissue was homogenized in a solution of RIPA lysis buffer/10% protease inhibitor. Protein concentration was determined using a Peterson Modified Lowry assay. Approximately 20 μg/ml of supernatants were loaded and separated by 8-12% SDS-PAGE gels. The proteins were transferred to nitrocellulose membranes and incubated with primary antibodies in a block solution containing 3% BSA/2% milk/0.05% sodium azide in TBS/Tween overnight at 4° C. at the following dilutions: Rabbit anti-GFAP polyclonal antibody—1:1000 (Z0334, DAKO, Milan, Italy); mouse monoclonal IgG1 anti-βactin—1:100 (sc-8432, Santa Cruz Biotechnology, Santa Cruz, Calif.); mouse monoclonal IgG1 anti-MBP—1:1000 (SMI-99; Sternberger Monoclonals Inc, Lutherville Md.); and mouse monoclonal anti-MBP (mAb381, Chemicon, Temecula, Calif.). After rinsing, blots were incubated with either horseradish peroxidase (HRP)-conjugated goat anti-rabbit IgG—-1:1000 (55676, MP Biomedicals, Irvine, Calif.) or HRP-conjugated goat anti-mouse IgG—1:1000 (674281, ICN Biomedicals, Irvine, Calif.) rinsed and developed with Super Signal West Pico Chemiluminescent detection reagents (Pierce, Rockford Ill.). β-actin expression was used as an internal loading control.

### Example 8

### Preparation of Dissociated Cultures of Spinal Cord Cells

To compare the in vitro development of oligodendrocyte lineage cells between WT and Cxcr2^{-/-} animals, dissociated cell cultures of post-natal day 8 (p8) spinal cord were established as previously described (Warf et al., J. Neurosci. 11:2477-2488 (1991)) and labeled after 2 days in vitro with mAb O4 to identify proligodendrocytes and mAbO1 to identify newly differentiated oligodendrocytes (Sommer and Schachner, Dev. Biol. 83:311-327 (1981)). The relative number of each different cell type in relationship to the total number of DAPI positive cells was counted by an observer blinded to the phenotype from 10 randomly selected fields taken from at least 2 different coverslips from 2 different preparations. The data were pooled and presented as mean+/−standard deviation.

### Statistical Analyses and Plots

Data was analyzed using one-way ANOVA and means comparison using Bonferroni post-test on Origin software. Statistical significance was set to p<0.05. Values are described in the text as mean+/−standard deviation followed by the real p-value, and plotted either as means+/−standard deviation or mean+/−two standard errors of the mean. In all plots significance is indicated by asterisks where one asterisk means p<0.05, two asterisks mean p<0.01 and three asterisks mean p<0.001.

### Example 9

### Altered PLP-EGFP+ Cell Density in Cxcr2^{-/-} Mice

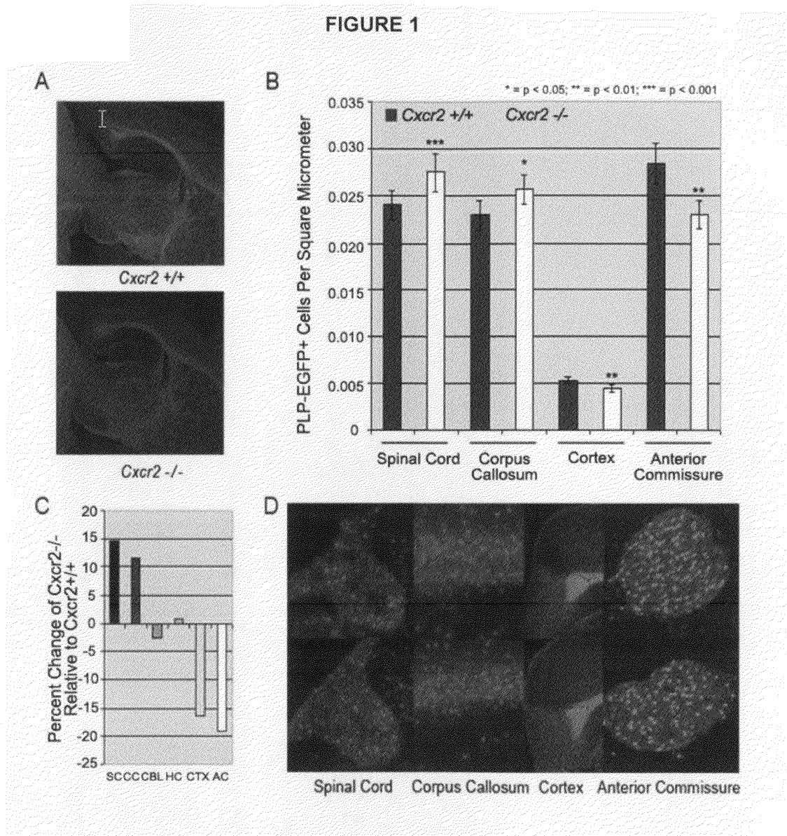

To assess the effects of CXCR2 deficiency on oligodendrocyte lineage cells and myelination, Cxcr2^{-/-} mice (Cacalano et al., Science 265:682-684 (1994)) were crossed to PLP-EGFP+transgenic mice (Mallon et al., J. Neurosci. 22:876-885 (2002)) allowing for the identification of oligodendrocyte lineage cells by green fluorescence. Comparison of multiple regions of white and gray matter from Cxcr2^{-/-}:PLP/DM20-EGFP+ mice and WT littermates demonstrated a generalized reduction in the intensity of PLP fluorescence in Cxcr2^{-/-} animals (FIG. 1A) indicating a reduction in the number of oligodendrocytes or an alteration in the intensity of PLP expression.

Quantitation of PLP-EGFP+ oligodendrocyte lineage cells in WT and Cxcr2^{-/-} mice revealed localized relative differences in the density of labeled cells in both the brain and spinal cord. Specifically, an increase in the density of PLP-EGFP+ cells was observed in the corpus callosum (Cxcr2^{+/+}=0.00229+/−0.00024, Cxcr2^{-/-}=0.00256+/−0.00024, p=0.00189; Mean+/−SD, p-Val) and spinal cord (Cxcr2^{+/+}=0.00240+/−0.00055, Cxcr2^{-/-}=0.00275+/−0.00073, p=0.00059; Mean+/−SD, p-Val) of Cxcr2^{-/-} animals (FIGS. 1B,C,D). A decrease was observed in the posterior cingulate cortex (Cxcr2^{+/+}=0.00054+/−0.00005, Cxcr2^{-/-}=0.00045+/−0.00004, p=0.00314; Mean+/−SD, p-Val) and the anterior commissure (Cxcr2^{-/-}=0.00284+/−0.00031, Cxcr2^{-/-}=0.00230+/−0.00021, p=0.00004; Mean+/−SD, p-Val) of Cxcr2^{-/-} animals (FIGS. 1B,C,D). No difference was observed in the density of PLP-EGFP+ cells between Cxcr2^{-/-} and WT mice in the hippocampus or corpus callosum (data not shown, p=0.75175, p=0.41963, respectively).

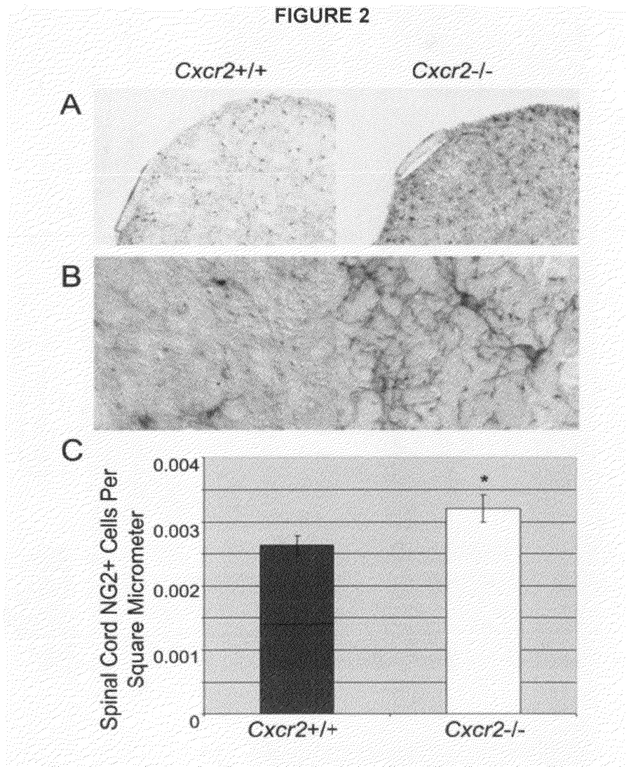

To selectively compare the density of OPCs in WT and Cxcr2^{-/-} spinal cords, the relative density of NG2 cells was assayed. Spinal cords of Cxcr2^{-/-} animals contained significantly more NG2+ cells per area than sex matched WT littermates (Cxcr2^{+/+}=0.00263+/−0.00013, Cxcr2^{-/-}=0.00321+/−0.00018, p=0.01126; Mean+/−SD, p-Val, FIG. 2C). Furthermore, the intensity of NG2 labeling and the size and ramifications of NG2+ cell processes were dramatically increased in Cxcr2^{-/-} animals (FIGS. 2A,B) and the cells were more concentrated towards the pial surface (FIG. 2A). Taken together these data indicate that CXCR2 contributes to establishing numbers of oligodendrocyte lineage cells and may do so in a regional fashion. In addition CXCR2 can affect the capacity of OPCs to differentiate into more mature lineage stages or modulate the availability of OPCs.

### Example 10

### Decreased Growth and Premature Death of Cxcr2^{-/-} Mice

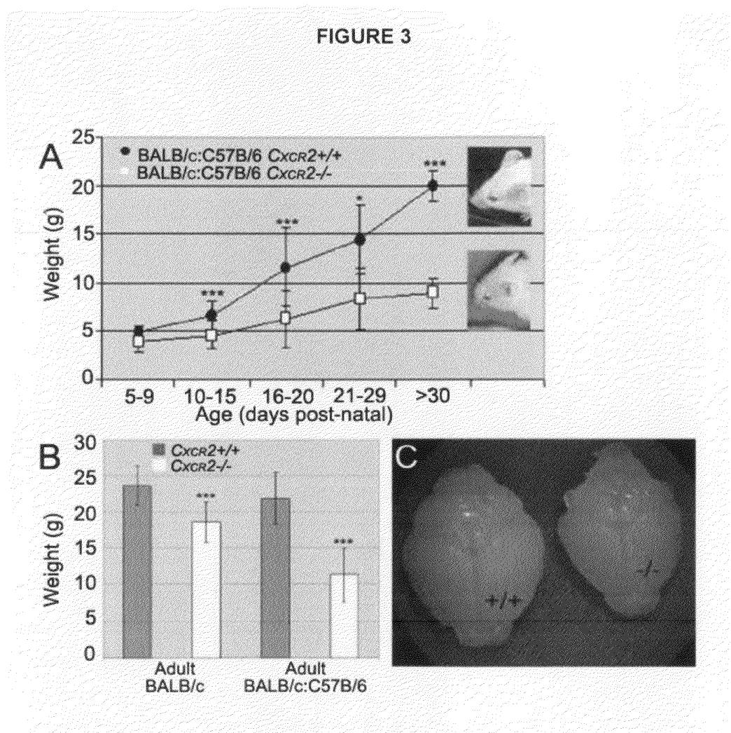

The generation of the Cxcr2^{-/-}:PLP/DM20-EGFP+ mice revealed strain differences. Upon crossing BALB/c Cxcr2^{-/-} mice (which had no obvious gross phenotype) to PLP-EGFP animals on a C57BL/6 background, we observed that the mixed background (BALB/c:C57BL/6) Cxcr2^{-/-} animals exhibited a significant decrease in weight gain (p10-15: Cxcr2^{+/+}=6.62+/−1.40, Cxcr2^{-/-}=4.64+/−1.53, p=0.00; Mean+/−SD, p-Val, FIG. 3A) with approximately 80% dying before P15 (10 times more than wild-type). No increased mortality rate was observed in heterozygous, as compared to WT mice. Cxcr2^{-/-} mice in the BALB/c:C57BL/6 mixed background which did not express EGFP also showed this weight and mortality phenotype demonstrating that it is independent of EGFP expression. BALB/c:C57BL/6 Cxcr2^{-/-}:PLP/DM20-EGFP+ mice were obtained from heterozygous sibling crosses as null animals were unable to reproduce. Most Cxcr2^{-/-} animals in this mixed background demonstrated reduced brain weight, size (FIG. 3B) and altered facial features (FIG. 3A) as well as changes in stance, gait, and lordosis (data not shown). In contrast, despite lordosis BALB/c Cxcr2^{-/-} animals were fertile and did not exhibit such marked weight differences or increased mortality rates compared to controls.

### Example 11

### Reduced Spinal Cord White Matter Area in Cxcr2^{-/-} Mice

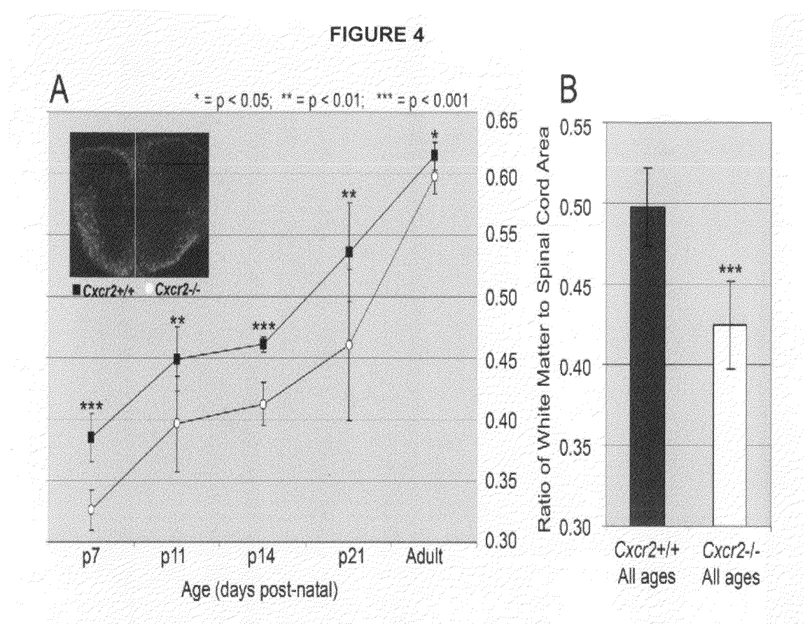

In neonatal animals CXCL1/CXCR2 signaling influences spinal cord OPC proliferation and migration. (Robinson et al., Neurosurgery 48:864-874 (2001); Robinson et al., J. Neurosci. 18:10457-10463 (1998); Tsai et al., Cell 110:373-383 (2002); Wu et al., J. Neurosci. 20:2609-2617 (2000)). To determine whether the lack CXCR2 resulted in persistent alterations in white matter formation, the relative area of the white matter was compared in matching spinal cord levels from Cxcr2^{-/-}:PLP/DM20-EGFP+ and sex matched WT littermates. Despite an increased density of fluorescent PLP-EGFP+ cells throughout the spinal cord, Cxcr2^{-/-} mice had decreased white matter area at all ages assayed. Differences in white matter area were detectable at the end of the first postnatal week, p7, (FIG. 4A) a time at which myelination is ongoing. For example, the p7 spinal cord sections in the insert in FIG. 4A show a ratio of 0.393 for the Cxcr2^{+/+} (left) and 0.313 for the Cxcr2^{-/-} (right), indicating a significant reduction in white matter area in Cxcr2 null animals. Differences in white matter area between Cxcr2^{-/-} and WT animals persisted throughout development into the adult although they were less apparent in the adult (p7: Cxcr2^{+/+}=0.385+/−0.020, Cxcr2^{-/-}=0.326+/−0.016, p=0.000; Adult: Cxcr2^{+/+}=0.614+/−0.011, Cxcr2^{-/-}=0.598+/−0.014, p=0.024; Mean+/−SD, p-Val; FIG. 4A plot). Therefore, although the spinal cords of the Cxcr2^{-/-} animals contained increased numbers of oligodendrocyte lineage cells the relative area of spinal cord white matter was reduced (FIG. 4B). Furthermore, the differences were more pronounced during early postnatal development, indicating that Cxcr2^{-/-} mice develop at a delayed rate, and partially recover during maturation.

### Example 12

### Hypomyelination in Spinal Cord White Matter of Cxcr2^{-/-} Mice

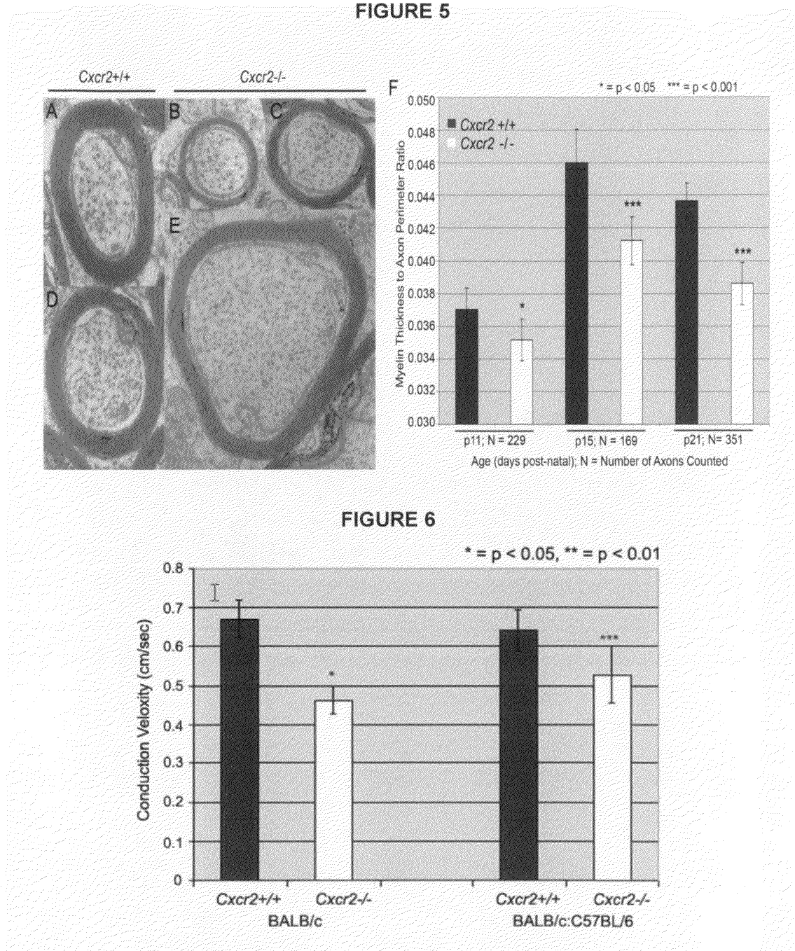

Several factors may have accounted for the reduced white matter in Cxcr2^{-/-} animals including a reduction in the number of mature oligodendrocytes, reduced thickness of myelin or numbers of axons. Given the increased density of NG2+ and PLP-EGFP+ cells in Cxcr2^{-/-} mice, a reduction in oligodendrocyte numbers was unlikely. To determine whether Cxcr2^{-/-} animals had alterations in axonal size or number, or a reduction in myelin formation, electron microscopic (EM) analyses of the spinal cords of Cxcr2^{-/-} and sex-matched littermate WT controls were performed. At all ages evaluated, a reduction in the pseudo-G-ratio was observed in Cxcr2^{-/-} animals, regardless of axonal size (FIG. 5). Although the axons in FIGS. 5B and 5C are only slightly smaller than those in FIGS. 5A and 5D the number of turns of myelin around them differed greatly. Axons in 5B and 5C were surrounded by 4 and 6 myelin turns respectively, while those in 5A and 5D were surrounded by 11 and 12 myelin wraps. Even larger axons in Cxcr2^{-/-} animals, such as that shown in 5E with only 10 wraps, frequently had relatively thin myelin compared to those seen on Cxcr2^{+/+}. Similar differences in the pseudo-G-ratio between Cxcr2^{-/-} and WT animals were detected in both BALB/c (data not shown) and BALB/c:C57B1/6 animals. This hypomyelination was detectable as early as p11 (Cxcr2^{+/+}=0.037+/−0.010, Cxcr2^{-/-}=0.035+/−0.010, p=0.037; Mean+/−SD, p-Val) and was maintained throughout development (p15: Cxcr2^{+/+}=0.047+/−0.010, Cxcr2^{-/-}=0.041+/−0.011, p=0.000; p21: Cxcr2^{+/+}+=0.044+/0.010, Cxcr2^{-/-}=0.039+/−0.012, p=0.000; Adult (BALB/c): Cxcr2^{+/+}=0.058+/−0.017, Cxcr2^{-/-}=0.054+/−0.017, p=0.022; Mean+/−SD, p-Val, FIG. 5F). Reduced myelin thickness was not associated with altered axonal diameters between Cxcr2^{-/-} and WT animals (data not shown, p=0.238-0.809). Estimates of the total number and proportions of myelinated and unmyelinated axons in homologous regions of the spinal cord revealed no marked differences between Cxcr2^{-/-} and WTs animals, although there was a trend towards more myelinated and less unmyelinated axons per unit area in WT animals (data not shown, p=0.094).

### Example 13

### Decreased Central Conduction Velocity

To assess if these structural differences resulted in a physiological phenotype, spinally elicited evoked potential recordings were performed on animals with the mixed background. Reduced central conduction velocity of lumbar evoked potentials was observed in 2-3 month old BALB/c:C57BL/6 mice (Cxcr2^{+/+}+=0.670+/−0.037, Cxcr2^{-/-}=0.461+/−0.032, p=0.000; Mean+/−SD, p-Val; FIG. 6, left). To ensure these differences were not a consequence of the systemic changes in Cxcr2^{-/-} animal of the mixed strain, the study was repeated with 2-4 month old BALB/c mice and similar results were obtained (Cxcr2^{+/+}=0.0643+/−0.043, Cxcr2^{-/-}=0.527+/−0.072, p=0.013; Mean+/−SD, p-Val; FIG. 6, right). There was no statistically significant difference between Cxcr2^{+/+} mice in the BALB/c and mixed background (p=0.273) or between Cxcr2^{-/-} mice in the BALB/c and mixed background (p=0.082). These data show that the absence of CXCR2 results in functional deficits in the adult nervous system and that these changes are intrinsic to the nervous system and independent of the systemic effects on weight and size observed in the mixed background. No significant differences were seen between CXCR2 null and WT animals for evoked potential amplitudes (data not shown, p=0.154), indicating that reductions in central conduction velocity are not associated with intrinsic axonal defects.

### Example 14

### Ultrastructural Analysis of Myelin and Paranodal Junctions

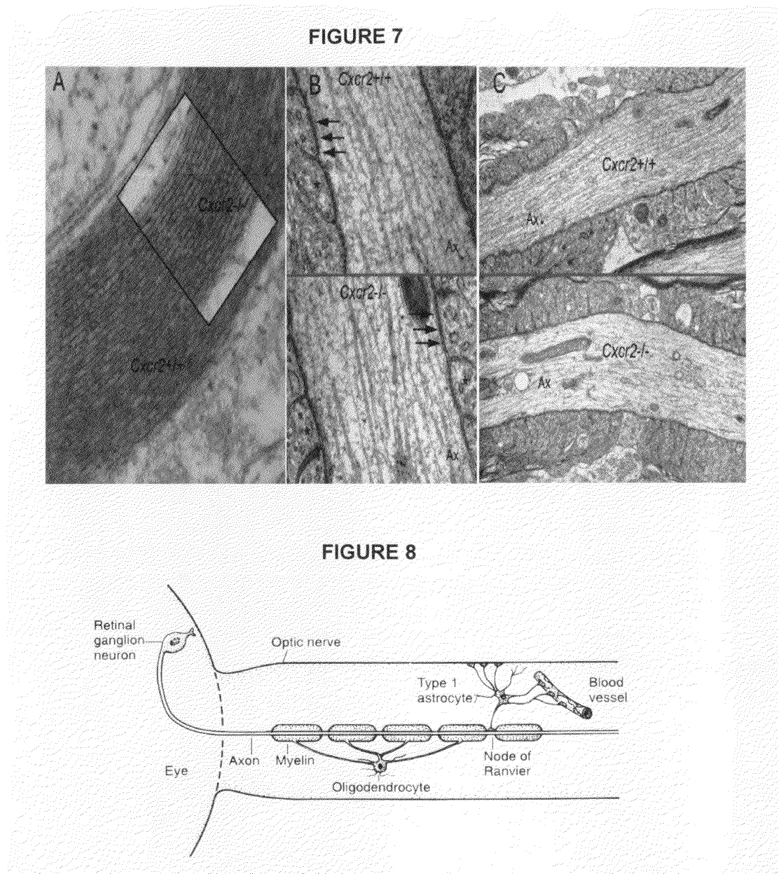

Conduction velocity deficits have been observed in mouse models with defective paranodal junction formation without alterations in myelin thickness or compaction (Bosio et al., Cell Tissue Res. 292:199-210 (1998); Boyle et al., Neuron 30:385-397 (2001); Popko, Glia 29:149-153 (2000)) as well as in mice with hypomyelination and alterations in myelin compaction (Neuberg et al., J. Neurosci. Res. 53:542-550 (1998)). To determine if alterations in axo-glial junction formation or myelin compaction may contribute to decreased conduction velocity in Cxcr2^{-/-} animals, the compaction and periodicity of myelin as well as the structural components of the paranodal junctions were analyzed. Electron microscopy studies revealed that Cxcr2^{-/-} myelin sheaths, although thinner, showed normal compaction and periodicity of major dense and intraperiod lines (FIG. 7A). Intact and well-oriented paranodal loops and transverse bands (FIG. 7B) were also seen at the paranodal junction in both Cxcr2^{-/-} and WT animals indicating proper axo-glial junction formation. Furthermore, no apparent differences in the size or organization of nodes of Ranvier were detected between Cxcr2^{-/-} and WT mice (FIG. 7C). These data indicate that the myelin formed in the Cxcr2 null animals is structurally normal but reduced in amount.

### Example 15

### Altered Glial Protein Expression

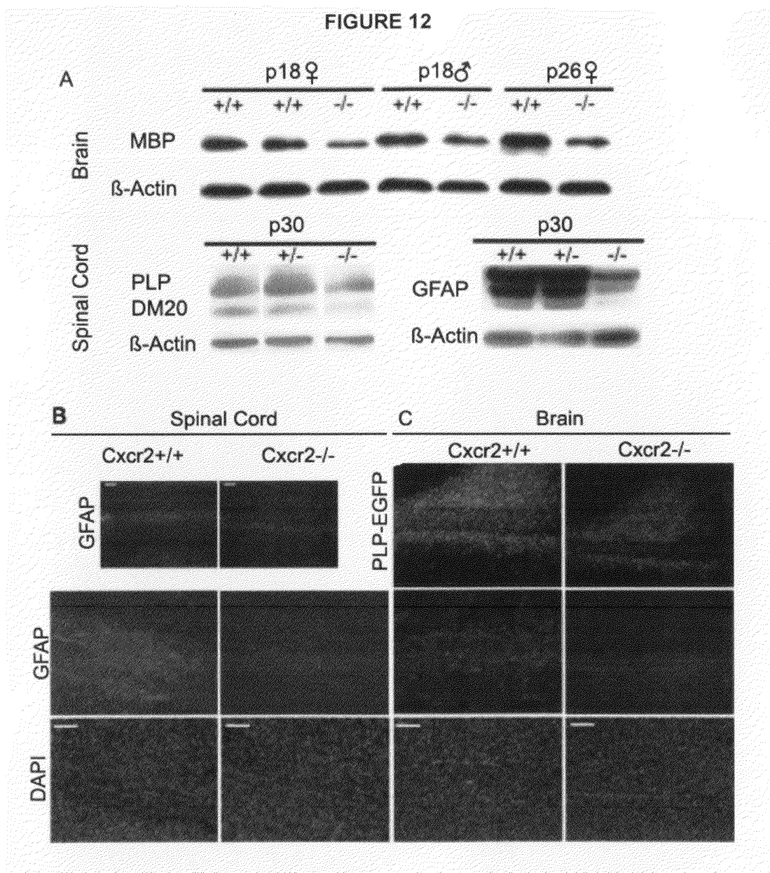

To analyze further the composition of white matter in Cxcr2^{-/-} spinal cord, protein expression of an abundant myelin protein, myelin basic protein (MBP), was compared by western blot analyses. Compared to WT, Cxcr2^{-/-} animals had reduced MBP levels (FIG. 12A) consistent with hypomyelination, and reduced PLP-EGFP expression. Interestingly, GFAP levels were reduced in Cxcr2^{-/-} animals as well (FIG. 12). As the majority of GFAP is produced by white matter astrocytes, this finding is consistent with reduced white matter in these animals.

Modulating CXCR2 signaling indirectly affects neural cells other than OPCs. The spinal cords of Cxcr2^{-/-} animals showed reductions in white matter area, GFAP, PLP, and MBP. During normal development, the expression of GFAP and MBP are frequently in register (Dziewulska et al., Folia Neuropathol 37:81-6 (1999); Landry et al., J. Neurosci. Res. 25:194-203 (1990); Takashima and Becker, Brain Dev. 6:451-457 (1984)), and GFAP null mice exhibit white matter structural abnormalities and hypomyelination (Liedtke et al., Neuron 17:607-615 (1996)). The establishment of normal white matter architecture reflects interactions between astrocytes and oligodendrocyte lineage cells, mediated in part through chemokine signaling.

### Example 16

### Altered Oligodendrocyte Differentiation in Spinal Cords Cultures from Cxcr2^{-/-} Mice

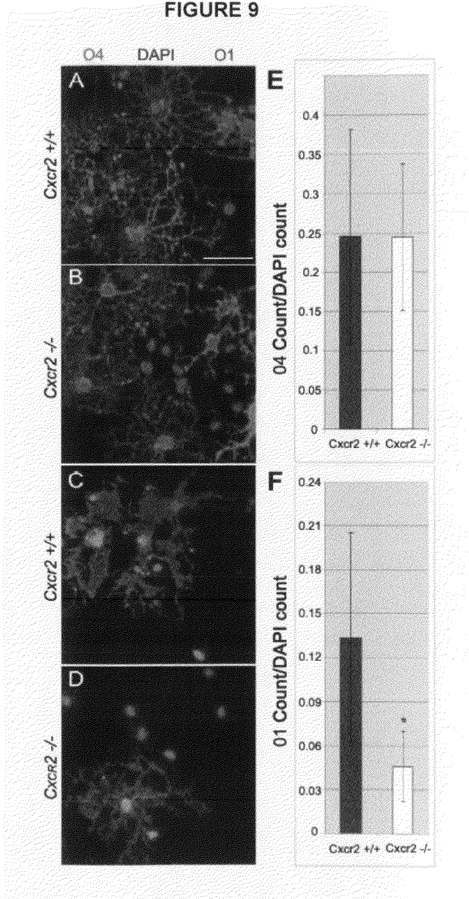

To determine whether the differences in oligodendrocyte lineage cell numbers were a consequence of systemic changes or alterations within the CNS, cultures of p8 Cxcr2^{-/-} and WT littermate mice were established and the development of oligodendrocytes assayed. After 2 days in vitro, the number of O4+OPCs was not significantly different (Cxcr2^{+/+}+=0.245+/−0.137, Cxcr2^{-/-}=0.244+/−0.093, p=0.977; Mean+/−SD, p-Val) in cultures of Cxcr2^{-/-} (FIGS. 9B,E) and WT animals (FIGS. 9A,E). By contrast, a significant reduction in the number of O1+differentiated oligodendrocytes (Cxcr2^{+/+}=0.133+/−0.071, Cxcr2^{-/-}=0.046+/−0.024, p=0.000; Mean+/−SD, p-Val) was seen in the Cxcr2^{-/-} cultures (FIGS. 9D,F) when compared to WT (FIGS. 9C,F). The data indicate that the maturation of oligodendrocytes is perturbed in Cxcr2 null animals, offering an in vitro system in which to study mechanisms underlying the structural and functional changes observed in the adult CNS.

### Example 17

### Assay of a Modulator of CXC-Mediated Signaling for Promoting Remyelination

Demyelinating lesions are generated in adult rats through localized injection of LPC into the dorsal columns. The animals are allowed to survive up to 7 days post injury and transverse and longitudinal slice cultures are prepared. Purified labeled OPCs are injected into the slice adjacent to, or into the lesion area, and their behavior monitored, thus following cell migration in this system. To test the effects of blocking Cxcr2 on injected OPC behavior, control slices are grown in regular medium and experimental slices grown in the presence of different concentrations of a CXCR2 inhibitor (e.g., repertaxin). The behavior of the injected cells are compared under the two conditions. One control experiment is to inhibit CXCL1 enhanced, PDGF driven proliferation of purified OPCs by drug addition and assay to establish approximate dose regimes. In addition, another control experiment uses anti Cxcr2 antibodies that have been shown to be effective in previous studies.

In the absence of Cxcr2 inhibitors, injected OPCs will migrate towards the lesion but stop in adjacent tissue and proliferate, based on finding that elevated levels of CXCL1, and OPCs in regions adjacent to lesions (Omari et al., Glia, 53:24-31 (2006)). In the presence of inhibitors of Cxcr2, substantially more migration results, toward the lesion and thus promoting pre-myelinating cells to infiltrate the lesion area. In developmental studies removing Cxcr2 signaling resulted in more extensive OPC migration in spinal cord slice cultures.

Some control and treated cultures are maintained for longer to assay the level of remyelination in the lesion(s). Multiple assays are done on a single lesioned animal since each slice is independent. The post lesion interval and dose at which a CXC-signaling inhibitor (e.g., repertaxin) is most effective may be determined thereby facilitating the more complex in vivo studies. All studies are done on multiple slices taken from at least three different lesioned animals to control for inter-lesion/animal variation

### Example 18

### Assay the Effect of Inhibitors of Cxcr2 on Repair of Demyelinating Lesions

Local demyelination lesions are generated in the dorsal columns of adult rats and at an appropriate post insult interval (1-5 days) an osmotic minipump delivering appropriate doses of a bioactive agent (e.g., CXC-signaling inhibitor) is surgically installed on the back of the animal. The pump delivers one or more drugs for 24-48 hrs or as needed and the animals recover for 7-10 days. Control animals receive minipumps with vehicle alone. One positive control is the use of the commercially available function blocking anti-Cxcr2 antibody used in previous studies. Repair is assessed my comparing lesion size in 3 dimensional reconstruction of bioactive agent animal and control treated animals. Candidate therapeutics are selected based on smaller lesions observed (e.g., remyelination).

### Example 19

### Treatment with Modulators of CXC-Mediated Signaling to Enhance Remyelination

An effective amount of a CXCR2-targeting bioactive agent is administered to a subject suffering from a demyelinating disorder (e.g., MS). For example, repertaxin is administered using dosages determined based on the subject's weight and/or stage of disease. Repertaxin is administered to a subject, either directly or systemically, which dosage can include from about 1, 2, 5, 7, 10, 12, 15, 17, 20, 25, 30, 35 or 40 mg/kg (mg repertaxin/kg body weight). Furthermore, multiple administration of a dose can occur on a daily, weekly or monthly periodicity. For example, multiple administration can include 1, 2, 3, 4, 5, 6, 7, 8, 9, 10, 11, 12, 13, 14, 15, 16, 17, 18, 19, 20, 25, 30 or 40 administrations daily, weekly or monthly.

Efficacy is measured using standard processes familiar to one of ordinary skill in the art, including MRI imaging, physical dexterity, or other clinical scoring methods. (See, e.g., Noseworthy et al., Curr. Opin. Neurol. 10:201-210 (1997)).

### Example 19

### PLP Expression Levels Reflect Alterations in Myelin and are Drastically Reduced in Cxcr2^{+/+}, But not in Cxcr2^{-/-} Mice after Cuprizone Exposure

MBP and PLP are important structural proteins of myelin. Previous studies have shown a temporal correlation between the events that characterize demyelination and remyelination after cuprizone treatment and the expression of myelin related genes (Jurevics et al., J. Neurochem. 82:126-136 (2002); Matsushima and Morell, Brain Pathol. 11:107-116 (2001)). These studies (Jurevics et al., J. Neurochem. 82:126-136 (2002); Matsushima and Morell, Brain Pathol. 11:107-116 (2001)) suggest an association between demyelination and decreased expression of PLP and MBP genes, and between their subsequent increases in expression and remyelination.

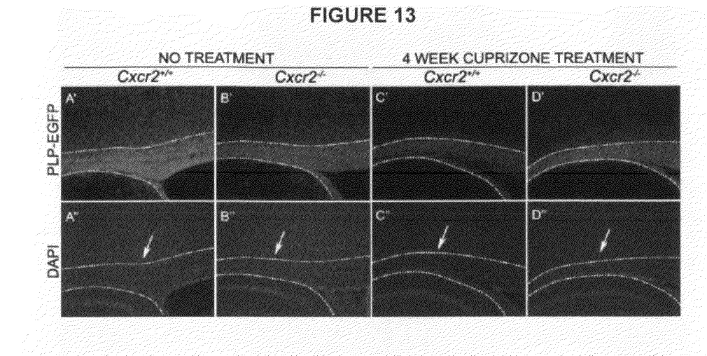

MBP and PLP expression are intrinsically reduced in Cxcr2^{-/-} mice, which exhibit hypomyelination (Padovani-Claudio et al., Glia 54:471-483 (2006)). Consequently in the absence of cuprizone treatment, Cxcr2^{-/-} mice (FIG. 13B′) had lower expression of PLP-EGFP than did their respective Cxcr2^{+/+} sex matched littermate controls (FIG. 13 A′). When the PLP-EGFP expression in brains of Cxcr2^{+/+} and Cxcr2^{-/-} mice was compared after 4 weeks of cuprizone treatment, however, there was a significant reduction in the intensity of PLP-EGFP in the corpus callosum of Cxcr2^{+/+} mice (FIG. 13C′). At all experimental time points evaluated, this reduction was evident in both the number of cells that expressed the EGFP reporter in Cxcr2^{+/+} mice, as well as in the intensity of the EGFP signal, and these changes correlated with decreased cellularity of the corpus callosum (FIG. 13C″, compare to 13A″,B″, and D″). The reduction seen in Cxcr2^{+/+} mice after treatment was evident in relation to both PLP levels of Cxcr2^{+/+} mice before treatment (FIG. 13 A′) and of Cxcr2^{-/-} mice before (FIG. 13B′) and after treatment (FIG. 13D′). There was no change in the expression of PLP in Cxcr2^{-/-} mice (compare FIG. 13B′ to D′).

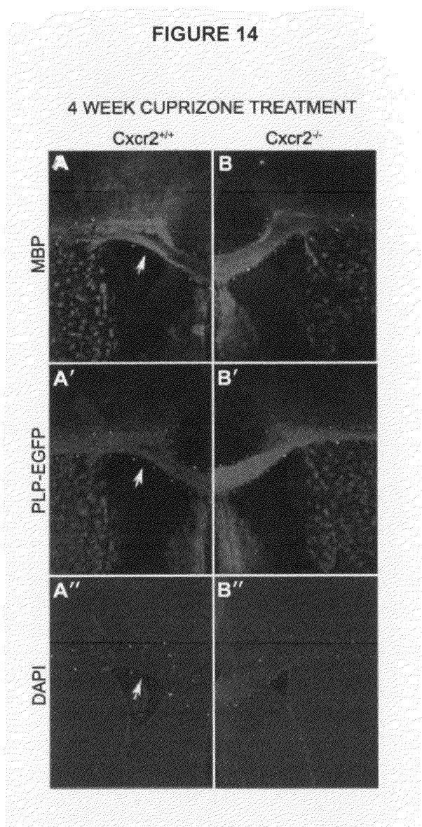

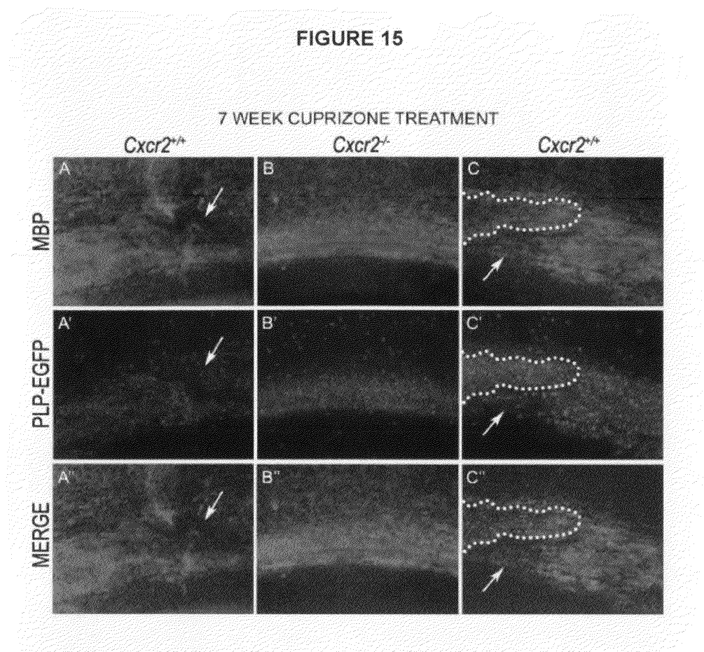

Areas of demyelination, evident by lack of MBP staining, were seen in the Cxcr2^{+/+} (FIG. 14A, arrow) but not in the Cxcr2^{-/-} mice (FIG. 14B) at the 4 week experimental time-point. These demyelination foci correlated with foci of decreased PLP expression (FIG. 14A′, arrow) and decreased cellularity (FIG. 14A″, arrow) in Cxcr2^{+/+} mice. The correlation between decreased PLP expression, myelin loss and hypo-cellularity at 4 weeks is consistent with reports of mature oligodendrocyte apoptosis, myelin gene down-regulation, and demyelination after cuprizone treatment (Matsushima and Morell, Brain Pathol. 11:107-116 (2001)). The fact that these changes are not seen in the Cxcr2^{-/-} mice, suggest that the lack of CXCR2 may confer protection from damage to oligodendrocytes and myelin, or promote oligodendrocyte survival in these mice. Even after 7 weeks of cuprizone treatment, extensive destruction of myelin could be frequently noted in tissue from Cxcr2^{+/+} mice (FIGS. 15A, A″ and 15C,C″), but not in tissue from Cxcr2^{-/-} mice (FIGS. 15B,B″). The loss of myelin commonly correlated with loss of PLP expression (FIGS. 15A,A′, arrow and 15C,C′,C″, arrow). In some occasions, however, intensely green PLP-EGFP+ cells were seen in areas where there was pronounced MBP depletion in Cxcr2^{+/+} mice (FIGS. 15C,C′, dotted area) indicating, in correspondence with the established sequence of events induced by cuprizone exposure in mice (Matsushima and Morell, Brain Pathol. 11:107-116 (2001)), that oligodendrocytes may be attempting to carry out remyelination.

### Example 20

### Unlike Cxcr2^{+/+} Mice, Cxcr2^{-/-} Mice do not Exhibit Prominent Astrogliosis after Exposure to Cuprizone

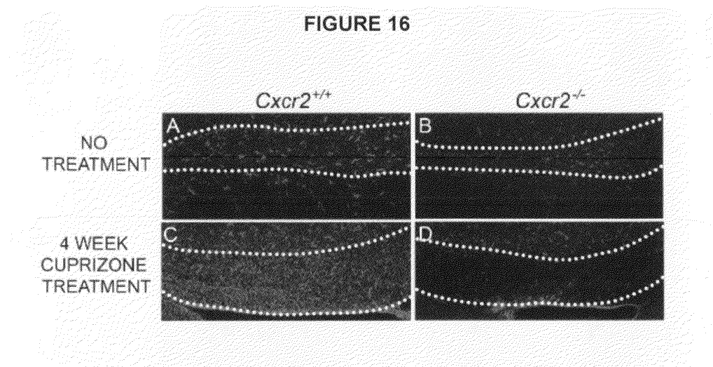

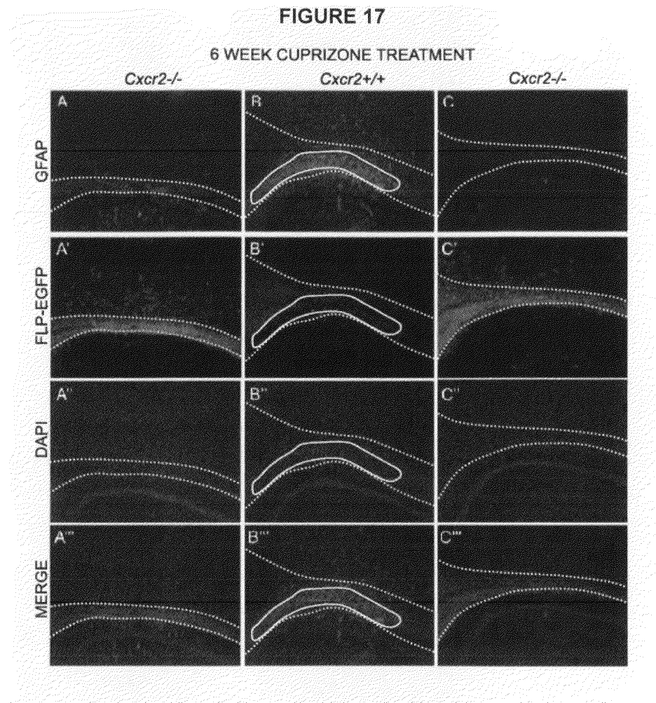

In addition to decreased PLP expression, as reported previously, Cxcr2^{-/-} mice fed a normal diet had lower expression of GFAP than did their respective Cxcr2^{+/+} controls (Padovani-Claudio et al., Glia 54:471-483 (2006)). GFAP is a marker for astrocytes and its upregulation is associated with astrogliosis. Astrocytes and microglia are usually upregulated after 3 weeks of cuprizone treatment and this upregulation tends to peak by 5 weeks of cuprizone exposure (Matsushima and Morell, Brain Pathol. 11:107-116 (2001)). Consistent with this, the levels of GFAP in Cxcr2^{+/+} mice (FIG. 16A), which were already higher than in Cxcr2^{-/-} mice before treatment (FIG. 16B), exhibited further upregulation after the initiation of treatment (FIG. 16C). Astrocytes appeared to be more reactive, with an apparent increase in their numbers as well as alterations in their morphology, changes which were most pronounced within the corpus callosum (FIG. 16C). The Cxcr2^{-/-} mice, in contrast, did not display significant changes in GFAP expression after treatment with cuprizone, and only few astrocytes were found within the corpus callosum in these mice (FIG. 16D). A similar profile of changes in GFAP expression was observed at 6 weeks in both Cxcr2^{-/-} (FIGS. 17A,C) and Cxcr2^{+/+} mice (FIG. 17B). The increase in GFAP expression in wild-type mice (FIG. 17B) correlated with decreased PLP-EGFP expression (FIG. 17B′) and with increased cellularity (FIG. 17B″) of the corpus callosum, indicating the infiltration of astrocytes into the corpus callosum where oligodendrocytes used to reside before demyelination (FIG. 17B′″, compare to A′″ and C′″). Astrocytic scars are associated with neuropathological states. Therefore, the lack of astrocyte activation in conjunction with preservation of PLP and MBP expression in Cxcr2^{-/-} mice (FIGS. 16D, 17A,C) illustrates that there is protection from pathology or enhancement of the repair process in the absence of CXCR2 signaling.

### Example 21

### A Reduction in the Activation of Microglia is Observed in Cxcr2^{-/-} Mice after Cuprizone Exposure

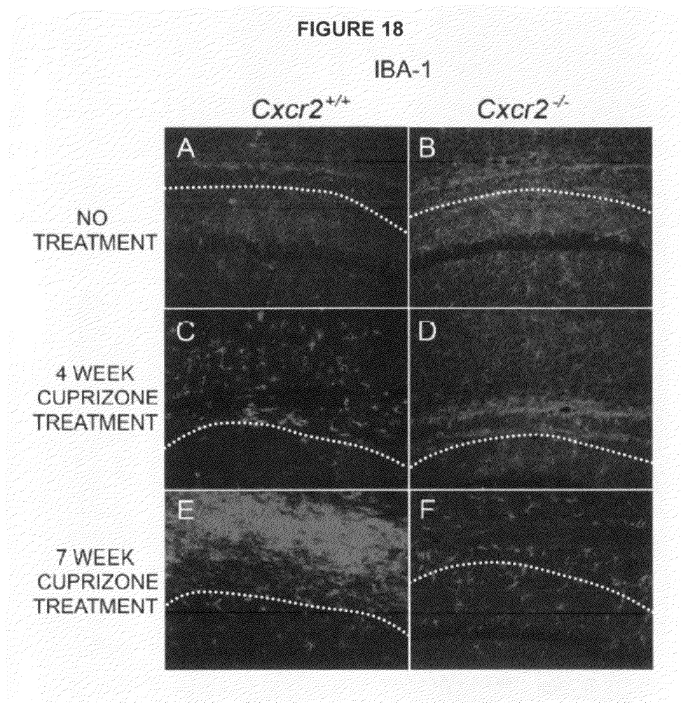

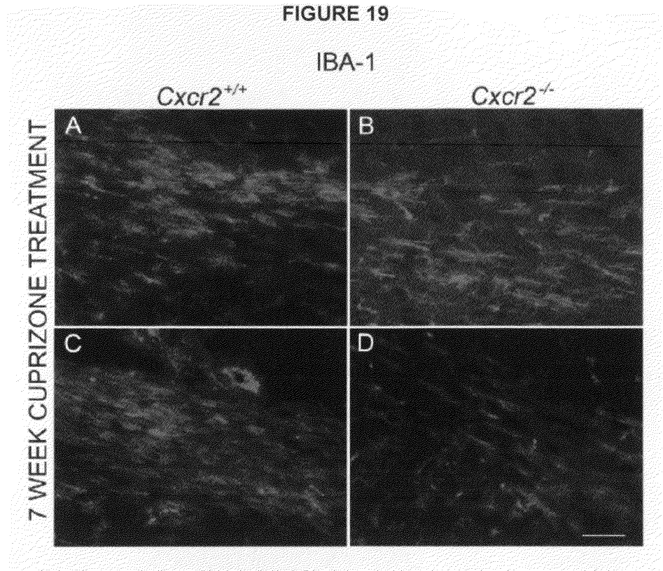

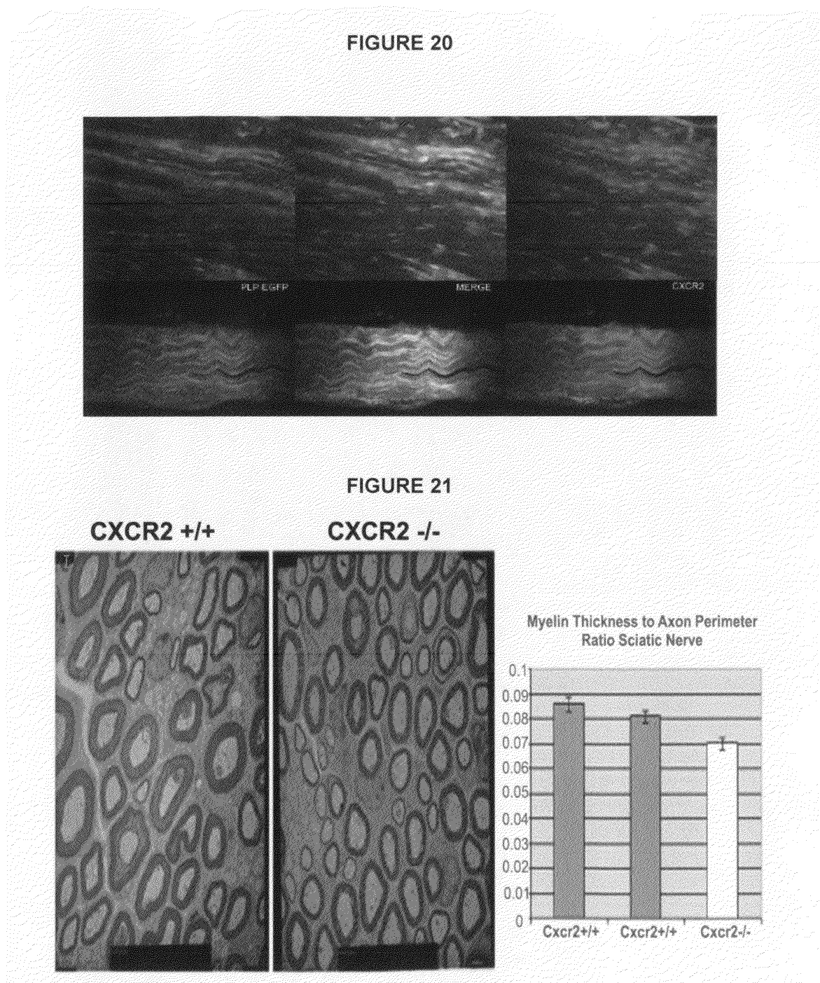

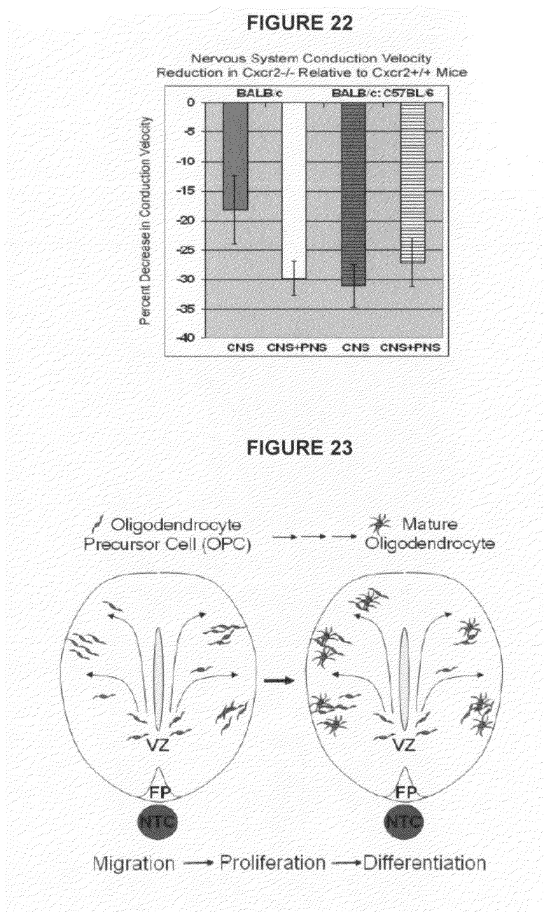

Microglia are phagocytic cells and their activation can lead to myelin destruction by phagocytosis. When a normal diet was fed to Cxcr2^{-/-} and Cxcr2^{+/+} mice, the levels of expression of the microglia/macrophage specific calcium binding protein IBA-1 were higher in Cxcr2^{-/-} mice (FIG. 18 B>A). Upon addition of cuprizone to the diet for 4 weeks, there was a gradual upregulation of IBA-1 in the Cxcr2^{+/+} (FIG. 18C) but not in the Cxcr2^{-/-} mice (FIG. 18D). By 7 weeks of cuprizone treatment the increase in EBA-1 expression and apparent microglial activation was dramatic in Cxcr2^{+/+} mice (FIG. 18E), while microglia appeared to be only mildly activated in Cxcr2^{-/-} mice (FIG. 18F). The increase in the activation of microglia was especially evident in the corpus callosum of Cxcr2^{+/+} mice, where microglia adopted a round morphology (FIGS. 18A,C). In Cxcr2^{-/-} mice, on the contrary, microglial upregulation was minimal, and most of the IBA-1+ cells exhibited long thin ramifications extending from their cytoplasm characteristic of quiescent microglia (FIGS. 18B,D).

### Example 22

### CXCL1 and CXCR2 Expression in Demyelinating Lesions

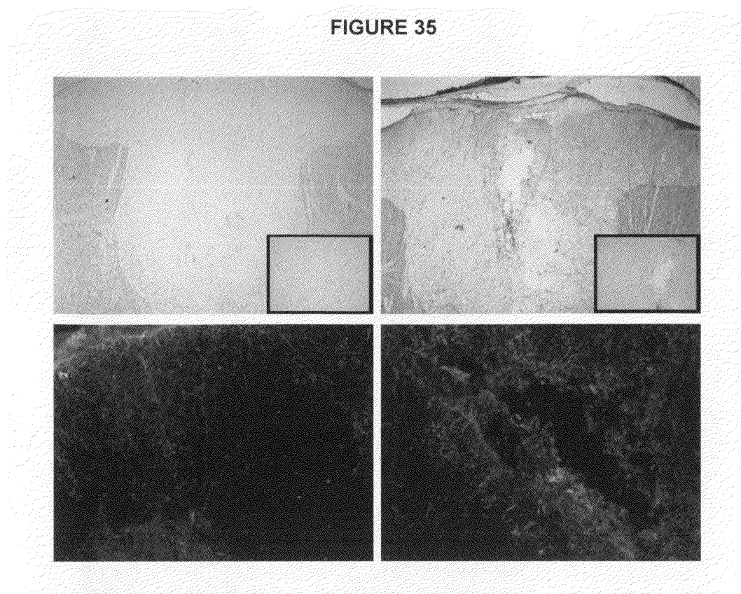

The dorsal columns of the adult (SD; female) rats were injected with 3≡1 of 1% lysolecithin (in NaCl) to induce local demyelinating lesions. Levels of CXCR2 and CXCL1 proteins were assayed and compared to glial fibrillary acidic protein (GFAP) expression in the injury area (FIG. 35). CXCL1 and CXCR2 expression is upregulated in local demyelinating lesions. The CXCR2 increase in expression appears to be associated with GFAP+ astrocytes in the lesion, but not outside the lesion.

### Example 23

### CXCL1 Effects on Astrocytes and Glial Scar Induction

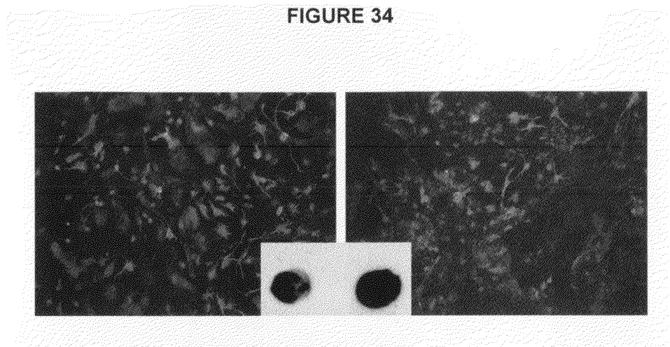

Addition of CXCL1 (0.5 ng/ml) to purified astrocyte cultures induces CSPG expression and changes morphology. Cells were treated with CXCL1 (0.5 ng/ml) for 3 days and assayed for CSPG deposition onto substrate by immunocytochemistry and released into medium. By dot blot (inset) CXCL1 increased CSPG (antibody to CSPG). GFAP (red), CSPG (green) (FIG. 34) For the culture studies, cultures of spinal cord astrocytes were assayed for basal levels of mRNA expression of CXCL1 and CXCR2 by RT-PCR (FIG. 33). The effect of 3 day CXCL1 protein addition (0.5 ng/ml) on CSPG protein expression was conducted on spinal cord astrocyte cultures as well as by immunocytochemistry and dot blot assays to analyze secreted and bound CSPG (FIG. 34).

### Example 24

### Neutralizing CXCR2 Antibody in Demyelinating Lesions of the Adult Spinal Cord and in Cell Culture

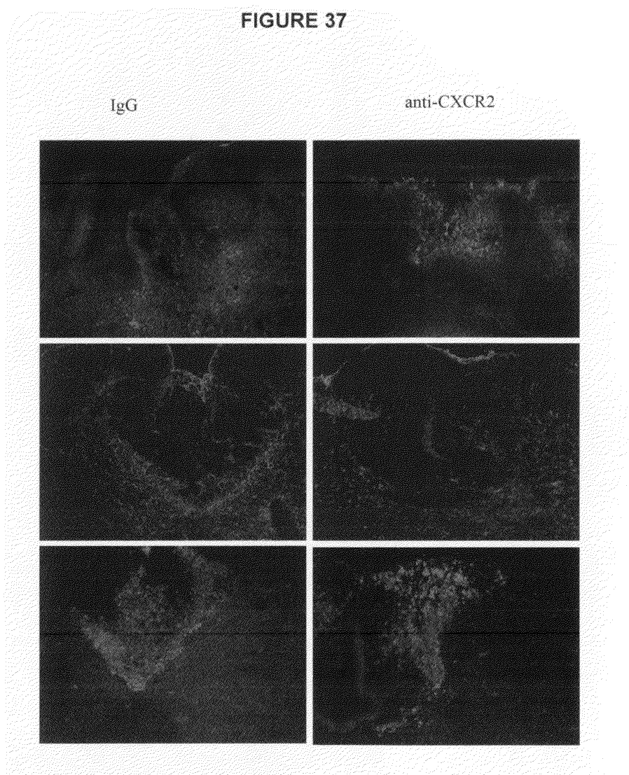

The dorsal columns of the adult (SD; female) rats were injected with 3≡1 of 1% lysolecithin (in NaCl) to induce local demyelinating lesions. Two days after lesion induction, 5≡1 of neutralizing antibody against CXCR2 or isotype IgG control antibodies (R&D Systems; 100≡g/ml) were injected locally into lesion area. The total survival time was ten days and spinal cords were sectioned and either processed for immunohistochemistry or histology. Entire lesion sections were obtained and stained with Luxol fast blue, a stain for myelin (FIG. 36). The lesion was reconstructed using 3D Doctor software program where lesion volume and 3 dimensional images were constructed (FIG. 36). Protein expression within the lesion was analyzed using standard immunohistochemistry techniques using antibodies for myelin basic protein (MBP), GFAP, ED1 (macrophage/microglia), and vimentin (FIG. 37).

### Example 25

### Assay of a Modulator of CXC-Mediated Signaling for Ameliorating Gliosis

The dorsal columns of the adult (SD; female) rats are injected with 1% lysolecithin (in NaCl) to induce local demyelinating lesions. Two days after lesion induction, candidate inhibitors (e.g., repertaxin, or compounds disclosed in FIG. 10) for ameliorating gliosis is injected locally into lesion area. Dosages are from about 0.01 mg to 500 mg V/kg body weight per day, e.g. about 20 mg/day. Spinal cords are sectioned and processed for immunohistochemistry or histology. Entire lesion sections are obtained and stained with Luxol fast blue, a stain for myelin. The lesion is reconstructed using 3D Doctor software program where lesion volume and 3 dimensional images are constructed. Protein expression within the lesion is analyzed using standard immunohistochemistry techniques using antibodies for myelin basic protein (MBP), GFAP, ED1 (macrophage/microglia), and vimentin. For the culture studies, cultures of spinal cord astrocytes are assayed for basal levels of mRNA expression of CXC receptors and/or ligands by RT-PCR. CSPG protein expression is detected by conducting on spinal cord astrocyte cultures immunocytochemistry and dot blot assays to analyze secreted and bound CSPG.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the claims herein define the scope of the invention and that methods and structures within the scope of these claims and their equivalents be covered thereby.

What is claimed is:

1. A method of ameliorating a neuropathy comprising administering to a subject in need thereof a therapeutically effective amount of a bioactive agent that selectively inhibits CXCR1 and/or CXCR2-mediated signaling relative to other CXC receptors as ascertained in a cell-based assay.

2. A method of promoting glial cell migration comprising contacting a glial cell with a bioactive agent that inhibits CXCR-mediated signaling in said glial cell, wherein said migration is increased as compared to a glial cell not contacted with said bioactive agent.

3. A method of promoting glial cell proliferation and/or differentiation comprising contacting a glial cell with a bioactive agent that selectively inhibits CXCR1 and/or CXCR2-mediated signaling relative to other CXC receptors as ascertained in a cell-based assay, wherein said proliferation and/or differentiation is increased as compared to a glial cell not contacted with said bioactive agent.

4. A method of promoting remyelination comprising administering to a subject exhibiting a demyelinating lesion with a bioactive agent, wherein said bioactive agent is effective in reducing gliosis through CXCR-mediated signaling, thereby promoting remyelination in said subject.

5. A method of ameliorating gliosis comprising administering to a subject in need thereof a therapeutically effective amount of a bioactive agent that selectively inhibits CXCR1 and/or CXCR2 mediated signaling relative to other CXC receptors as ascertained in a cell-based assay.

6. The method of claim 1, wherein said neuropathy is multiple sclerosis.

7. The method of claim 1, wherein said other CXC receptors are CXCR3 or CXCR4.

8. The method of claim 4, wherein said CXCR-mediated signaling is via CXCR1 and/or CXCR2.

9. The method of claim 1 or 5, wherein said bioactive agent directly binds CXCR1 and/or CXCR2.

10. The method of claim 2 or 4, wherein said bioactive agent inactivates CXCL1, CXCL2, CXCL3, CXCL5, CXCL7, or CXCL8.

11. The method of claim 1 or 5, wherein said bioactive agent reduces CXCR1 and/or CXCR2 activity.

12. The method of claim 2 or 4, wherein said bioactive agent is a peptide, polypeptide, antibody, antisense molecule, siRNA, small molecule or peptidomimetic.

13. The method of claim 1, 2, 3, 4, or 5, wherein said bioactive agent is selected from the group of compounds in FIGS. 10A, 10B, 10C and 11.

14. The method of claim 1 or 4, wherein the bioactive agent reduces expression of GFAP, vimentin, heparan sulphate proteoglycan (HSPG), dermatan sulphate proteoglycan (DSPG), keratan sulphate proteoglycan (KSPG), or chondroitin sulphate proteoglycan (CSPG).

15. The method of claim 2 or 3, wherein said glial cell is selected from a group consisting of oligodendrocyte, oligodendrocyte progenitor, Schwann, astrocytes, microglial and a combination thereof.
